(19) United States
(12) Patent Application Publication
(10) Pub. No.: US 2022/0233512 A1
(43) Pub. Date: Jul. 28, 2022

(54) INHIBITING AN IMMUNE RESPONSE MEDIATED BY ONE OR MORE OF TLR2, RAGE, CCR5, CXCR4 AND CD4

(71) Applicant: Cassava Sciences, Inc., Austin, TX (US)

(72) Inventors: Hoau-Yan Wang, Philadelphia, PA (US); Lindsay Burns Barbier, Austin, TX (US)

(21) Appl. No.: 17/517,879

(22) Filed: Nov. 3, 2021

Related U.S. Application Data
(60) Provisional application No. 63/109,213, filed on Nov. 3, 2020.

(51) Int. Cl. A61K 31/438; A61K 31/4709; A61K 31/4402; A61P 37/06; A61P 25/04; A61K 31/527

(52) U.S. Cl. CPC A61K 31/438; A61K 31/4709; A61K 31/527; A61P 37/06; A61P 25/04; A61K 31/4402

(57) ABSTRACT

A method of inhibiting an immune response mediated by one or more of TLR2, RAGE, CCR5, CXR4 and CD4 cell surface receptors of cells in recognized need is disclosed. The method contemplates administering to such cells an effective amount of a of a compound or a pharmaceutically acceptable salt thereof that binds to a pentapeptide of filamin A (FLNA) whose structure is defined within. In one embodiment, the cells to which the compound or its salt is administered exhibit a hyperinflammatory syndrome.

### CROSS-REFERENCE TO RELATED APPLICATION

This application claims priority from application Ser. No. 63/109,213 that was filed on Nov. 3, 2020, whose disclosures are incorporated herein by reference.

### TECHNICAL FIELD

The present invention contemplates a method of treatment that inhibits an immune response mediated by one or more of TLR2, HMGB1, CCR5, CXCR4 and CD4 receptors. The immune response to be inhibited is the induced release of one or more of the above inflammatory cytokines that at times can lead to pathogenic inflammation. The method and use also lead to the lessening of the severity of the immune response that can itself be life-threatening due to a hyperinflammatory syndrome such as sepsis, cytokine storm, hypotensive shock or multi-organ failure.

### BACKGROUND ART

Immune cells are activated by stressed or infected cells through receptor-ligand interactions. [Liu et. al., Cell Mol Immunol 13(1):3-10 (January 2016).] Involved receptors include the toll-like receptors (TLRs) such as TLR2, as well as cytokine receptors such as CCR5 and CXCR4, and also a T cell receptor, CD4. High mobility group box protein 1 (HMGB1) is a ligand for the cell surface receptor referred to as receptor for advanced glycation end products (RAGE).

Cytokines are a category of small proteinaceous molecules (about 5-20 kDa) important in cell signaling that cannot cross the cellular lipid bilayer to enter the cytoplasm. Cytokines are non-hormonal molecules known to be involved in autocrine, paracrine and endocrine signaling as immunomodulating agents.

Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells, with a given cytokine at times being produced by more than one type of cell. [Stedman's Medical Dictionary, 28th ed., Wolters Kluwer Health, Lippincott, Williams & Wilkins (2006).] Cytokines act through cell surface receptors and are especially important in the immune system where cytokines modulate the balance between humoral and cell-based immune responses, and help regulate the maturation, growth, and responsiveness of particular cell populations.

Chemokines represent a large group of structurally related chemotactic cytokines. [Struyf et al., Eur J Immunol 31:2170-2178 (2001); Hughes et al., FEBS J 285:2944-2971 (2018).] Their primary structural hallmark is the presence of four, conserved cysteine residues. The positioning of the first two cysteines from the N-terminus permits division of the family into CC and CXC chemokines, where “X” is one amino acid residue. CC chemokines are also sometimes referred to as beta-chemokines. The first two cysteine residues from the amino-terminus are adjacent to one another in CC chemokines. CXC chemokines contain a single amino acid residue between those two cysteines.

Most members of the CC chemokine family attract several leukocyte types, except neutrophils. The selectivity of chemokines for a certain leukocyte subset can be explained by the tightly regulated expression of ligand-specific G protein-coupled receptors [GPCRs].

CC chemokine receptor type 5, also known as CCR5 or CD195, is a G protein-coupled receptor on the surface of white blood cells. [Jiao et al., Cancer Res 79(19): 4801-4807 (2019 Oct. 1).] The CCR5 protein belongs to the beta-chemokine receptors family of integral membrane proteins. [Samson et al., Biochemistry-US 35(11): 3362-3367 (1999).]

Because chemokines are a type of cytokine, both will be referred to herein as chemokines for ease of discussion.

CCR5's cognate ligands include CCL3, CCL4 (also known as MIP 1α and 1β, respectively), and CCL3L1. [Miyakawa et al., J Biol Chem 277(7):4649-4655 (2002).] CCR5 furthermore interacts with CCL5 (a chemotactic cytokine protein also known as RANTES). [Struyf et al., Eur J Immunol 31:2170-2178 (2001); and Slimani et al., Biochim Biophys Acta 1617(1-2):80-88 (2003).] CCR5 is predominantly expressed on T cells, macrophages, dendritic cells, eosinophils, and microglia. CCR5 is a co-receptor with CD4 for HIV entry into T cells.

CXCR4 is an alpha-chemokine receptor specific for stromal-derived-factor-1 (SDF-1 also called CXCL12), a molecule endowed with potent chemotactic activity for lymphocytes. CXCR4 is another CD4 co-receptor that HIV can use to infect CD4⁺ T cells.

CXCR4 and its ligand SDF-1 were believed to be a relatively monogamous ligand-receptor pair (other chemokines are promiscuous, tending to use several different chemokine receptors). Recent evidence demonstrates ubiquitin is also a natural ligand of CXCR4 [Sani et al., J Biol Chem 285(20):15566-15576 (2010)], as is HMGB1 [Ran. et al., Mol Aspects Med 40:1-116 (December 2014)].

Ubiquitin is a small (76-amino acid residue) protein highly conserved among eukaryotic cells. It is best known for its intracellular role in targeting proteins for degradation via the ubiquitin proteasome system. Evidence in numerous animal models suggests ubiquitin is an anti-inflammatory immune modulator and endogenous opponent of proinflammatory damage associated molecular pattern molecules. [Majetschak J Leuko Biol 89(2):205-219 (2011).] It is speculated this interaction may be through CXCR4-mediated signaling pathways. MIF is an additional ligand of CXCR4. [Bernhagen et al., Nature Med 13(5):587-590 (2007).]

The cell surface receptor known as CD4 (cluster of differentiation 4) is a glycoprotein found on the surface of immune cells such as T helper cells, monocytes, macrophages, and dendritic cells. CD4⁺ T helper cells are often referred to as CD4 cells, T-helper cells or T4 cells. These T cells are referred to as helper cells because one of their main roles is to send signals to other types of immune cells, including CD8 killer cells, which then destroy infectious particles. If CD4 cells become depleted, for example in untreated HIV infection, or following immune suppression prior to a transplant, the body is left vulnerable to a wide range of infections that it would otherwise have been able to fight.

Human immunodeficiency virus (HIV)-1 infection requires envelope (Env) glycoprotein gp120− induces clustering of CD4 and coreceptors CCR5 or CXCR4 on the cell surface; this enables Env gp41 activation and formation of a complex that mediates fusion between Env-containing and target-cell membranes. Although mechanisms by which HIV-1 induces F-actin rearrangement in the target cell remain largely unknown, CD4 and the coreceptors are reported to interact with the actin-binding protein filamin-A, whose binding to HIV-1 receptors regulates their clustering on the cell surface. [Jiménez-Baranda et al., Nat Cell Biol 9:838-846 (2007).]

The CD4 D₁ domain interacts with the β₂-domain of MHC class II molecules. T cells displaying CD4 molecules (and not CD8) on their surface, therefore, are specific for antigens presented by MHC II and not by MHC class I (they are MHC class II-restricted). MHC class I contains beta-2 microglobulin.

CD4 is a co-receptor of the T cell receptor (TCR) complex and assists the latter in communicating with antigen-presenting cells. The TCR complex and CD4 bind to distinct regions of the antigen-presenting MHC class II molecule.

The short cytoplasmic/intracellular tail (C) of CD4 [Rudd et al., Proc Natl Acad Sci, USA 85(14):5190-5194 (1988)] contains a special sequence of amino acid residues that permit it to recruit and interact with the tyrosine kinase Lck. That interaction permits the tyrosine kinase Lck to phosphorylate tyrosine residues of nearby proteins to amplify the signal generated by the TCR and recruit and activate further protein tyrosine kinases (PTK) to further mediate downstream signaling through tyrosine phosphorylation. These signals lead to the activation of transcription factors, including NF-κB, NFAT, AP-1, to promote T cell activation. [Owens et al., Kuby Immunology (7th ed.) W.H. Freeman, New York, 100-101 (2013).] T-cells play a large part in autoinflammatory diseases. [Ciccarelli et al., Curr Med Chem 21(3):261-269 (2014).]

The receptor for advanced glycation end products (RAGE) was initially reported to bind the products of non-enzymatic glycation and oxidation of proteins/lipids, the advanced glycation end products, or AGEs [Schmidt et al., J Biol Chem. 267:14987-14997 (1992)]. RAGE is a key molecule in the onset and sustainment of the inflammatory response.

RAGE belongs to the superfamily of Ig type I cell-surface receptors and is expressed on all types of leukocytes promoting activation, migration, or maturation of the different cells. RAGE expression is prominent on the activated endothelium, where it mediates leukocyte adhesion and transmigration. Moreover, proinflammatory molecules released from the inflamed or injured vascular system induce migration and proliferation of smooth muscle cells (SMCs). [Kierdorf et al., J Leukoc Biol 94:55-68 (2013).]

It has been found that RAGE binds a diverse series of ligands beyond AGEs, such as members of the S100/calgranulin family, high mobility group box 1 (HMGB1), lysophosphatidic acid (LPA) and oligomeric forms of amyloid beta peptide (Ab) and islet amyloid polypeptide (IAPP) [Hoffman et al., Cell 97:889-901 (1999); Taguchi et al., Nature 405:354-360 (2000); Yan et al., Nature 382:685-691 (1996); Abedini et al., Rai et al., J Clin Investig 128:682-698 (2018); and Rai et al., J Exp Med. 209:2339-2350 (2012)]. These ligands bind to the extracellular domains of RAGE in a heterogeneous manner; although the extracellular V-type immunoglobulin (Ig) domain binds to many of the ligand families, the binding sites on the V-domain are multiple and spatially distinct.

In the absence of endogenous kinase activity, the means by which the RAGE cytoplasmic domain signals and impacts transcriptional programs and cellular functions remained elusive until the discovery that this RAGE intracellular domain binds the formin, Diaphanous1 (DIAPH1), and that this interaction is essential for RAGE signaling in multiple cell types [Hudson et al., J Biol Chem. 283:34457-34468 (2008)].

RAGE is a pattern recognition receptor (PRR) and central mediator of the innate immune response. Thus, RAGE recognizes self-derived molecules resulting from damaged cells, referred to as damage-associated molecules patterns (DAMPs) and microbe-specific molecular signatures known as pathogen-associated molecular patterns (PAMPs) that are both discussed further hereinafter. RAGE is also expressed on T and B lymphocytes, as well as on DCs, representing a new link between the innate and adaptive immune system.

RAGE-ligand interaction results in up-regulation of cytokines, chemokines, and adhesion molecules with possible roles in the commencement and continuation of inflammation [Rahimi et al., Cell Physiol Biochem 46:561-567 (2018)]. These authors noted an anti-inflammatory effect of sRAGE, but the pro-inflammatory effects of membrane-bound RAGE in MS that supports the association between IFNβ-1a treatment and increased sRAGE concentration.

Activation of RAGE is involved in the immediate inflammatory response. More importantly, perpetuation of RAGE signaling sustains the inflammation and leads to the establishment of chronic inflammatory disorders [Hudson et al., J Biol Chem. 283:34457-34468 (2008)].

A soluble form of RAGE that comprises the C-truncated RAGE without transmembrane and cytosolic domains also binds to RAGE ligands such as HMGB1. This C-truncated RAGE is referred to as soluble RAGE (sRAGE) [Raucci et al. FASEB J 22:3716-3727 (2008)].

sRAGE was reported to be an anti-inflammatory factor that mitigates the proinflammatory effects of HMGB1 in patients diagnosed with Guillain-Barré syndrome (GBS) by Zhang et al., Sci Rep 6:21890 (2016). Serum sRAGE levels were said to be lower only in patients with the acute motor axonal neuropathy (AMAN) subtype of GBS, whereas elevated serum HMGB1 levels were observed in all subtypes. Lower sRAGE and higher HMGB1 levels were said to may be related to the robust autoimmune response that underlies GBS, perhaps through increasing the release of inflammatory cytokines.

The interaction HMGB1 with TLR2, TLR4 and RAGE can ultimately lead to proliferation of the same cytokines. The identity of which of those three receptors are activated can be determined by which receptor(s) and/or their signalling proteins were expressed in an amount significantly greater than that normally present in non-activated cells of the same type. The presence or absence of these receptors is routinely assayed in body fluids such as serum or plasma using well-known techniques and commercial kits for measuring those receptors and signalling proteins.

The above immune cell and receptor interactions are those that occur in usual immune system functioning. Sometimes, those interactions go awry wherein the released cytokines induce white blood cells to continually activate more white blood cells to release more cytokines in a positive feedback loop [Lee et al., Blood 124(2):188-195 (July 2014)], such that the immune system over reacts causing what is referred to as cytokine storm syndrome, which is one form of a hyperinflammatory syndrome.

COVID-19 patients who undergo a hyperinflammatory syndrome such as cytokine storm and develop secondary hemophagocytic lymphohistiocytosis (sHLH), which causes acute respiratory distress syndrome (ARDS). ARDS causes about 50% mortality in these patients. Cytokine storms are seen in sepsis, non-infectious systemic inflammatory response syndrome (SIRS), macrophage activation syndrome (MAS), and secondary hemophagocytic lymphohistiocytosis.

Cytokine storm syndrome (CSS) also known as cytokine release syndrome (CRS) is a form of systemic inflammatory response syndrome (SIRS) that can be triggered by a variety of factors such as infections and certain drugs. CSS thus refers to an uncontrolled and overwhelming release of proinflammatory mediators by an overly activated immune system. [Lee et al., Blood 124:188-195 (2014).]

A Supplementary Appendix to Webb et al., Lancet Rheumatol published online Sep. 29, 2020, lists six categories of physiologic features said to be found in all hyperinflammatory syndromes, including that of COVID-19—1) fever, 2) macrophage activation, 3) hematologic dysfunction, 4) hepatic inflammation, 5) coagulopathy and 6) cytokinemia. Those six categories are well known, defined and assayed for in medical practice, and are used herein as criteria for a patient having a hyperinflammatory syndrome that is treatable by a method contemplated herein.

Fever is a cardinal feature of hyperinflammatory conditions, driven by IL-1, IL-6, TNF, prostaglandin secretion and other mechanisms. It is present in up to 89% of cases of symptomatic COVID-19, and is an important criterion for secondary hemophagocytic lymphohistiocytosis (sHLH), macrophage activation syndrome (MAS), and CRS. Excessive macrophage activation, unchecked by a dysfunctional cytotoxic T cell response, is the central mechanism in the pathophysiology of these hyperinflammatory disorders. In COVID-19, macrophage activation may be mediated by direct infection of macrophages and monocytes by SARS-CoV-2 as well as dysfunctional CD8 T cell IFN-γ response. Hyperferritinemia is an important surrogate for macrophage activation and a key criterion in sHLH and MAS diagnostic definitions. Elevated serum ferritin levels above 500 μg/L and 2000 μg/L, respectively, are included in criteria criteria for sHLH, whereas a threshold of 684 μg/L is used by the 2016 American College of Rheumatology criteria for MAS. Although frequently elevated in CRS, ferritin does not discriminate severity in CRS. In multiple COVID-19 case series, ferritin levels above 700 μg/L identify patients with an inflammatory phenotype and associated poor outcomes.

A clinically-based definition for CSS or CRS is provided in Tisoncik et al., Microbiol Mol Biol R 76(1):16-32 (March 2012). According to those authors, the cytokine storm is best exemplified by severe lung infections, in which local inflammation spills over into the systemic circulation, producing systemic sepsis, as defined by persistent hypotension, hyper- or hypothermia, leukocytosis or leukopenia, and often thrombocytopenia [citing: Levy et al. “2001 SCCM/ESICM/ACCP/ATS/SIS International Sepsis Definitions Conference” Crit Care Med 31:1250-1256 (2003)].

More recently, Teachey et al., Blood Advances, 4(20):5174-5183 (Oct. 27, 2020), sought to distinguish CRS from sepsis in children treated with chimeric antigen receptor T cells directed against CD19⁺ B cells. Those workers reported identifying 23 different cytokines that were significantly different between patients with sepsis and CRS. Using elastic net prediction modeling and tree classification, they identified cytokines that were able to classify subjects as having CRS or sepsis accurately. A markedly elevated interferon g (IFNg) or a mildly elevated IFNg in combination with a low IL1b were associated with CRS. A normal to mildly elevated IFNg in combination with an elevated IL1b was associated with sepsis.

In those studies, patients with IFNg levels greater than 83 μg/mL were classified as having CRS. Those patients with mildly elevated IFNg levels less than 83 μg/mL were also classified as having CRS if they had IL1b less than 8 μg/mL, whereas those patients with more than 8 μg/mL IL1b were classified as having sepsis. This combination of IFNg and IL1b was reported to categorize subjects as having CRS or sepsis with 97% accuracy.

Viral, bacterial, and fungal pulmonary infections all cause the sepsis syndrome, and these etiological agents are difficult to differentiate on clinical grounds. In some cases, persistent tissue damage without severe microbial infection in the lungs also is associated with a cytokine storm and clinical manifestations that mimic sepsis syndrome.

CSS is a common immunopathogenesis underlying many pathological processes, such as ARDS, sepsis, graft-versus-host disease (GvHD), macrophage activation syndrome (MAS) induced by rheumatic diseases, and primary and secondary hemophagocytic lymphohistiocytosis (HLH) [Mahajan et al., J Autoimmumn 100:62-74 (2019).]. Recently, CSS has also been reported to be a complication of immunotherapies, such as chimeric antigen receptor (CAR) T cell therapies. [Neelapu et al., Nat Rev Clin Oncol 15:47-62 (2014).] A review by Shimabukuro-Vornhagen et al. [J Immunother Cancer 6:56 (2018)], lists several instances of CSS reported in clinical drug trials in the years 2014-2018 using differing therapies on patients with different diseases.

CSS is broadly found in many areas of disease. Much of the recent work and publications dealing with CSS have addressed potential cellular and molecular mechanisms contributing to the cytokine storm in viral disease, some of which are specifically focused on influenza, and the diseases caused by the corona family of viruses that cause SARS, MERS and COVID-19. The following discussion focuses on the cytokine storm in the context of infection, with particular emphasis on respiratory viruses, and particularly SARS-CoV-2 the causative agent of COVID-19 disease, as illustrative of the causative pathogens and the cytokine storm that they induce in many of their victims.

For example, avian influenza A type H5N1 virus causes severe CSS disease in humans. Studies of H5N1-infected individuals revealed low peripheral blood T-lymphocyte counts and high chemokine and cytokine levels, particularly in those who died, and those findings correlated with pharyngeal viral loads. [de Jong et al., Nature Med 12(10):1203-1207 (October 2006).]

Previous experience with SARS and MERS has also revealed florid CSS in critically ill patients. Studies have shown that acute respiratory distress syndrome (ARDS) occurs in some SARS patients despite a diminishing viral load, suggesting that an exuberant host immune response rather than viral virulence is possibly responsible for tissue pathologies. Therefore, antiviral therapy alone may be inadequate. [Peirls et al., Lancet 361:1767-1772 (2003).] Corticosteroids, one of the most widely utilized anti-inflammatory agents, are still commonly prescribed in treating COVID-19 patients (72.2% in the ICU setting) [Wang et al., J Am Med Assoc 323:1061-1069 (2020).]

However, physicians need to be cautious of steroid use due to its nebulous benefits in the setting of viral respiratory infection. Several studies even reported inferior outcomes of SARS patients treated with corticosteroids. [Russell et al., Lancet 395:473-475 (2020).] Another concern of corticosteroids is their short- and long-term adverse effects. More than half of SARS patients treated with corticosteroids suffer from joint pain and bone marrow abnormalities. [Griffith et al., Radiology 235:168-175 (2005).]

Other therapies aiming to dampen excessive serum inflammatory mediators, such as plasmapheresis or continuous renal replacement therapy (CRRT), either require specific equipment or lack documented efficacy [Borthwick et al., Cochrane Database Sys Rev 1:CD008075 (2017).] Thus, there is still an unmet need for the treatment of pathogen-induced CSS. [Zumla et al., Lancet 395:e36-e36 (2020).]

In the past decade, immunotherapy has made great strides in managing CSS of various etiologies, including autoimmunity, malignancy and CAR T cell therapies. Bingwen Liu et al., [J Autoimmun doi.org/10.1016/j.jaut.2020.102452] proposed that attenuating the detrimental host immune response by immunomodulators may be a beneficial addition to antiviral therapy. Those authors proposed the use of the monoclonal antibody tocilizumab that binds to both soluble and membrane-bound IL-6 and has been approved by the U.S. Food and Drug Administration for the treatment of severe CAR T cell-induced CSS. [Neelapu et al., Nat Rev Clin Oncol 15:47-62 (2014).] Of course, such a monoclonal antibody has to be administered by infusion and cannot be given orally as by a tablet or capsule, causing a sterile hospital-like setting to be used for administration.

The innate immune system employs germline-encoded pattern-recognition receptors (PRRs) for the initial detection of microbes. PRRs recognize microbe-specific molecular signatures known as pathogen-associated molecular patterns (PAMPs) and self-derived molecules resulting from damaged cells, referred as damage-associated molecules patterns (DAMPs). PRRs activate downstream signaling pathways that lead to the induction of innate immune responses by producing inflammatory cytokines, such as type I interferon (IFN), and other mediators.

These processes not only trigger immediate host defensive responses such as inflammation, but also prime and orchestrate antigen-specific adaptive immune responses. [Janeway et al., Annu Rev Immunol 20:197-216 (2002).] These responses are essential for the clearance of infecting microbes as well as crucial for the consequent instruction of antigen-specific adaptive immune responses.

Mammals have several distinct classes of PRRs including Toll-like receptors (TLRs), RIG-I-like receptors (RLRs), Nod-like receptors (NLRs), AIM2-like receptors (ALRs), C-type lectin receptors (CLRs), and intracellular DNA sensors such as cGAS. Among these, TLRs were the first to be identified, and are the best characterized.

The TLR family comprises 10 members (TLR1-TLR10) in humans and 12 (TLR1-TLR9, TLR11-TLR13) in mice. TLRs localize to the cell surface or to intracellular compartments such as the endoplasmic reticulum (ER), endosome, lysosome, or endolysosome, and they recognize distinct or overlapping PAMPs such as lipid, lipoprotein, protein, and nucleic acid. Each TLR is composed of an ectodomain with leucine-rich repeats (LRRs) that mediate PAMPs recognition, a transmembrane domain, and a cytoplasmic Toll/IL-1 receptor (TIR) domain that initiates downstream signaling.

The ectodomain displays a horseshoe-like structure, and TLRs interact with their respective PAMPs or DAMPs as a homo- or heterodimer along with a co-receptor or accessory molecule [Botos et al., Structure (2011) 19:447-459 (2011).] Upon PAMPs and DAMPs recognition, TLRs recruit TIR domain-containing cytoplasmic adaptor proteins such as myeloid differentiation primary response protein (MyD88) and TIR-domain-containing adapter-inducing interferon-β (TRIF), which initiate signal transduction pathways.

Those pathways culminate in the activation of nuclear factor kappa-light-chain-enhancer of activated B cells (NF-κB), interferon-regulatory factor proteins (IRFs), or mitogen-activated protein (MAP) kinases (MAPKs) to regulate the expression of cytokines, chemokines, and type I interferons (IFNs) that ultimately protect the host from microbial infection. Recent studies have revealed that proper cellular localization of TLRs is important in the regulation of the signaling, and that cell type-specific signaling downstream of TLRs determines particular innate immune responses. [Kawasaki et al., Front Immunol 5: Article 461 (September 2014).]

TLRs are largely classified into two subfamilies based on their localization: cell surface TLRs and intracellular TLRs. Cell surface TLRs include TLR1, TLR2, TLR2, TLR5, TLR6, and TLR10, whereas intracellular TLRs are localized in the endosome and include TLR3, TLR7, TLR8, TLR9, TLR11, TLR12, and TLR13. [Kawasaki et al., Front Immunol 5: Article 461 (September 2014).]

Individual TLRs differentially recruit members of a set of TIR domain-containing adaptors such as MyD88, TRIF, TIR domain containing adaptor protein (TIRAP), or translocating chain-associated membrane protein (TRAM). MyD88 is utilized by all TLRs except TLR3, and activates NF-kB and MAPKs for the induction of inflammatory cytokine genes. TIRAP is a sorting adaptor that recruits MyD88 to cell surface TLRs such as TLR2 and TLR4, and through some endosomal TLRs.

TIRAP conducts the signal from the TLR to MyD88, and TRAM mediates the signal from the TLR to TRIF. TLR signaling is thus largely divided into two pathways, depending on the adaptor usage: the MyD88-dependent and TRIF-dependent pathways. [Kawasaki et al., Front Immunol 5: Article 461 (September 2014).]

Cell surface TLRs mainly recognize microbial membrane components such as lipids, lipoproteins, and proteins. TLR4 recognizes bacterial lipopolysaccharide (LPS). TLR2, as a homodimer, or as a heterodimer along with TLR1 or TLR6, recognizes a wide variety of PAMPs including lipoproteins, peptidoglycans, lipotechoic acids, zymosan, and mannan. TLR5 recognizes bacterial flagellin. TLR10 is pseudogene in mouse due to an insertion of a stop codon, but human TLR10 collaborates with TLR2 to recognize ligands from listeria. TLR10 can also sense influenza A virus infection. [Kawasaki et al., Front Immunol 5: Article 461 (September 2014).]

TLR2, sometimes designated CD282 (cluster of differentiation 282), is a protein that in humans is encoded by the TLR2 gene. As an illustrative MyD88-dependent pathway membrane surface receptor, TLR2 recognizes many bacterial, fungal, viral, and certain endogenous substances. In general, this results in the uptake (internalization, phagocytosis) of bound molecules by endosomes/phagosomes and in cellular activation.

Thus, such elements of innate immunity as macrophages, polymorphonuclear cells (PMNs) and dendritic cells assume functions of nonspecific immune defense, B1a and MZ B cells form the first antibodies, and specific antibody formation gets started in the process. Cytokines participating in this innate immune defense include tumor necrosis factor-alpha (TNF-α) and various interleukins (IL-1α, IL-1β, IL-6, IL-8, IL-12).

TLR2 is involved in the recognition of a wide array of microbial molecules representing broad groups of species such as Gram-positive and Gram-negative bacteria, as well as mycoplasma and yeast. TLR2 recognizes cell-wall components such as peptidoglycan (PGN), lipoteichoic acid (LTA) and lipoprotein from gram-positive bacteria, lipoarabinomannan from mycobacteria, and zymosan from yeast cell wall.

Illustratively, Wang et al., [Infect Immun 69:2270-2276 (2001)] reported that Gram positive bacteria (micrococci) and their peptidoglycan portions induced TLR2-dependent activation of the gene for IL-8 via a multi-membered signal transducing pathway. The variety of TLR2 ligands is the greatest among all the TLRs and this is due to the heterodimerization needed for most TLR2-mediated responses.

In 2002, the SARS viral pandemic broke out in Southern China. A rapid response from scientists identified a novel coronavirus as the causative agent of SARS, named SARS-Coronavirus (SARS-CoV) and angiotensin converting enzyme 2 (ACE2) as the human receptor of the virus. [Li et al., Nature 2003, 426:450-454 (2003).]

TLR2 has been shown to recognize the glycoproteins B and H of human cytomegalovirus (HCMV), the glycoproteins gH/gL and gB of herpes simplex virus (HSV), the UTPase of Epstein-Barr virus (EBV), the hemagglutinin protein of measles virus, the nsp4 of rotavirus, and the core and NS3 proteins of hepatitis C virus (HCV), mediating NF-κB activation and the subsequent induction of proinflammatory cytokines. In addition, TLR2 also has been implicated in mediating host responses to infections by vaccinia virus, lymphocytic choriomeningitis virus, varicella zoster virus, respiratory syncytial virus (RSV), although the exact viral PAMPs for TLR2 were not identified. Further pathogens whose immunogenic responses are TLR2-mediated include Gram-positive bacteria, Streptococcus B, Staphylococcus aureus, Treponema maltophilum, Wolbachia, Borrelia burgdorferi, Staphylococcus epidermis, Mycobacterium tuberculosis, Pseudomonas aeruginosa, measles virus, Herpes virus, Saccharomyces cerevisiae, Candida albicans and Trypanosoma cruzi. [Lester et al., J Mol Biol 426:1246-1264 (2014); Kim et al., BMB Rep 47(4):184-191 (2014); Mukherjee et al., Braz j infect dis 20(2):193-204 (2016); and Hijano et al., Front Immunol 10:Article 566 (March 2019).]

ACE-2 is a type I transmembrane metallocarboxy-peptidase with homology to ACE, an enzyme long-known to be a key player in the renin-angiotensin system (RAS) and a target for the treatment of hypertension. [Riordan, Genome Bio. 4:225 (2003).] ACE-2 is mainly expressed in vascular endothelial cells, the renal tubular epithelium, and in Leydig cells in the testes. [Kuba et al., Pharmacol. Ther. 128:119-128 (2010); Jinag et al., Nat. Rev. Cardiol. 11:413-426 (2014).] PCR analysis revealed that ACE-2 is also expressed in the lung, kidney, and gastrointestinal tract, tissues shown to harbor SARS-CoV. [Ksiazek et al., N. Engl. J. Med. 348:1953-1966 (2003); Harmer et al., FEBS Lett. 532:107-110 (2002); and Leung et al., Gastroenterology 125:1011-1017 (2003).]

The major substrate for ACE-2 is angiotensin II. [Tikellis et al., Int. J. Pept. 2012:256294 (2012).] ACE-2 degrades angiotensin II thereby, negatively regulating RAS. [Kuba et al., Pharmacol. Ther. 128:119-128 (2010); and Tikellis et al., Int. J. Pept. 2012:256294 (2012).] ACE-2 has also been shown to exhibit a protective function in the cardiovascular system and other organs. [Kuba et al., Pharmacol. Ther. 128:119-128 (2010).]

Although the above and other hyperinflammatory syndromes can be different in origin, treatment and outcome, their propagation to life-threatening disease state can be mediated by one or more of the cell surface receptors TLR2, CCR5 and CXCR4, CD4 and RAGE. Those mediating cell surface receptors themselves interact with and their signalling activities are mediated by the cytoskeletal protein, filamin A as is discussed hereinbelow.

Filamins [FLNs] are a family of cytoskeletal proteins—filamins A (FLNA) and B, but not C—that are expressed in non-muscle cells. These proteins were first reported in 1975 as the first non-muscle actin-binding protein [Hartwig et al., J Biol Chem 250:5696-5705 (1975); Wang et al., Proc Natl Acad Sci USA 72:4483-4486 (1975)].

Human FLNA is given the identifier P21333 in the UniProtKB/Swiss-Prot data base, and contains a sequence of 2647 amino acid residues (about 280 kDa). This protein is also sometimes referred to in the art as actin-binding protein (ABP-280). [Gorlin et al., J Cell Biol 111:1089-1105 (1990).]

The FLNA protein anchors various transmembrane proteins to the actin cytoskeleton and serves as a scaffold for a wide range of cytoplasmic signalling proteins. Filamins are essential for mammalian cell locomotion and act as interfaces for protein-protein interaction [van der Flier et al., Biochim Biophys Acta 1538:99-117 (2001)]. Besides its role in cell motility, FLNA is increasingly found to regulate cell signalling by interacting with a variety of receptors and signalling molecules. [Stossel et al., Nat Rev Mol Cell Biol 2:138-145 (2001); Feng et al., Nat Cell Biol 6:1034-1038 (2004)].

The FLNA protein consists of an N-terminal actin-binding domain (ABD) and a rod-like domain of 24 immunoglobulin-like (Ig) repeats (each about 96-amino acid residues long and numbered from the N-terminus), interrupted by two 30-amino acid residue flexible loops or hinges. The loop designated H1 is between repeats 15 and 16, and the loop designated H2 is located between repeats 23 and 24 [Gorlin et al., J Cell Biol 111:1089-1105 (1990); van der Flier et al., Biochim Biophys Acta 1538:99-117 (2001)].

H1 and H2 can be cleaved by calpains and caspases [Gorlin et al., J Cell Biol 111:1089-1105 (1990); Browne et al., J Biol Chem 275:39262-39266 (2000)]. Cleavage at H1 occurs between amino acid residues 1761 and 1762, and results in an about 170 kDa fragment consisting of the ABD and repeats 1-15 (IgFLNa-R1-15), plus an about 100 kDa polypeptide fragment consisting of repeats 16-24 (IgFLNa-R16-24).

It is noted that IgFLNa-R16-24 is said to have a mass of about 100 kDa in Loy et al., Proc Natl Acad Sci, USA, 100(8):4562-4567 (2003). That about 100 kDa polypeptide (IgFLNa-R16-24) is further cleaved at H2 to yield an about 90 kDa fragment that contains repeats 16-23 (IgFLNa R16-23) [Gorlin et al., J Cell Biol 111:1089-1105 (1990); van der Flier et al., Biochim Biophys Acta 1538:99-117 (2001)].

FLNA promotes orthogonal branching of actin filaments and links actin filaments to membrane glycoproteins. Filamin A is dimerized through the carboxy-terminal repeat (repeat 24) near the transmembrane regions, providing an intracellular V-shaped structure that is critical for function.

Each V-shaped FLNA dimer has two antiparallel self-bound domains 24 forming the apex of the “V”, and the remaining domains stretched out much like beads on a string with each of their N-terminal ABD portions bound to an actin molecule. More recently, it has been reported that C-terminal to the ABD, rod segment 1 (IgFLNa-R1-15) forms an extended linear structure without obvious inter-domain interactions. Rod segment 2 (IgFLNa-R16-23) assumes a compact structure due to multiple inter-domain interactions in which domains 16-17, 18-19 and 20-21 form paired structures. [Heikkinen et al., J Biol Chem, 284:25450-25458 (2009); Lad et al., EMBO J, 26:3993-4004 (2007).]

Proteolysis of FLNA is regulated in part by its phosphorylation on Ser 2152 (S2152) in repeat 20 (IgFLNa-R20), which is reported to render the full-length protein stable and resistant to cleavage. [Gorlin et al., J Cell Biol 111:1089-1105 (1990).]

Loy et al., Proc Natl Acad Sci, USA, 100(8):4562-4567 (2003), report that a H1 cleavage product containing repeats 16-24 and having a molecular weight of about 100 kDa co-localized with the androgen receptor to the nucleus in prostate cancer cells. Those workers noted that FLNA is generally regarded as a cytoplasmic architectural molecule. They characterized their finding of an additional function of its about 100 kDa polypeptide as a nuclear regulator of the androgen receptor to be “entirely unexpected” (at page 4565).

The about 100 kDa FLNA fragment found in a cellular nucleus is not phosphorylated on pS2152. Indeed, phosphorylation on pS2152 blocks the cleavage of FLNA in a prostate cancer line. [Garcia et al., Arch Biochem Biophys 446:140-150 (2006); Gorlin et al., J Cell Biol 111:1089-1105 (1990)].

As a key regulator of the cytoskeleton network, FLNA interacts with many proteins involved in cancer metastasis, [Yue et al., Cell & Biosci 3:7 (2013)] as well as in many other conditions. Thus, Nakamura et al., Cell Adh Migr. 5(2):160-169 (2011), discuss the history of research concerning FLNA and note that the protein serves as a scaffold for over 90 binding partners including channels, receptors, intracellular signaling molecules and transcription factors.

Phosphorylation has become recognized as a global regulator of cellular activity, and abnormal phosphorylation is implicated in a host of human diseases, particularly cancers. Phosphorylation of a protein involves the enzymatically-mediated replacement of an amino acid side chain hydroxyl of one or more serine, threonine or tyrosine residues with a phosphate group (—OPO₃⁻²).

Phosphorylation and its reverse reaction, dephosphorylation, occur via the actions of two key enzyme types. Protein kinases phosphorylate proteins by transferring a phosphate group from a nucleotide triphosphate such as adenosine triphosphate (ATP) or guanosine triphosphate (GTP) to their target protein. This process is balanced by the action of protein phosphatases, which can subsequently remove the phosphate group.

The amount of phosphate that is bonded to a protein at a particular time is therefore determined by the relative activities of the particular one or more associated kinase and phosphatase enzymes specific to that protein and to the particular amino acid residue(s) undergoing phosphorylation/dephosphorylation. If the phosphorylated protein is an enzyme, phosphorylation and dephosphorylation can impact its enzymatic activity, essentially acting like a switch, turning it on and off in a regulated manner. Phosphorylation can similarly regulate non-enzymatic protein-protein interactions by facilitation of binding to a partner protein.

Protein phosphorylation can have a vital role in intracellular signal transduction. Many of the proteins that make up a signaling pathway are kinases, from the tyrosine kinase receptors at the cell surface to downstream effector proteins, many of which are serine/threonine kinases.

FLNA is phosphorylated at a number of positions in its protein sequence in both normal and in diseased cells such as cancer cells. For example, the enzyme PAK1 (EC 2.7.11.1) is a protein kinase of the STE20 family that regulates cell motility and morphology. FLNA phosphorylation at position 2152 by PAK1 is required for PAK1-mediated actin cytoskeleton reorganization and for PAK1-mediated membrane ruffling. [Vadlamudi et al., Nat. Cell Biol. 4:681-690 (2002); Woo et al., Mol Cell Biol. 24(7):3025-3035 (2004).] Cyclin B1/Cdk1 (EC:2.7.11.22; EC:2.7.11.23) phosphorylates serine 1436 in vitro in FLNA-dependent actin remodeling. [Cukier et al., FEBS Letters 581(8):1661-1672 (2007).]

The UniProtKB/Swiss-Prot data base entry for human FLNA (No. P21333) lists published reports of the following amino acid residue positions as being phosphorylated under different circumstances: 11, 1081, 1084, 1089, 1286, 1338, 1459, 1533, 1630, 1734, 2053, 2152, 2158, 2284, 2327, 2336, 2414, and 2510. Further, polyclonal and monoclonal antibodies are commercially available from one or more of Abgent, Inc. (San Diego, Calif.), Abcam, Inc. (Beverly, Mass.), Bioss, Inc. (Woburn, Mass.), and GeneTex, Inc. (Irvine, Calif.) that immunoreact with FLNA that is phosphorylated (phospho-FLNA) at serine-1083, tyrosine-1046, serine-1458, serine-2152, and serine-2522.

Recent work published by two of the inventors and co-workers, Wang et al., Neurobiol. Aging 55:99-114 (2017), showed for the first time that amyloid-β₁₋₄₂ (Aβ₄₂) triggers a conformational change in the FLNA scaffolding protein to induce FLNA associations with α7-nicotinic acetylcholine receptor (α7nAChR) and TLR4. These aberrant associations respectively enable Aβ₄₂'s toxic signaling via α7nAChR to hyperphosphorylate tau protein, and TLR4 activation to release inflammatory cytokines. Sumifilam, previously referred to as PTI-125 and as Compound C0105M or as Compound C0105, is a small molecule that preferentially binds conformationally-altered FLNA and restores its native conformation, restoring receptor and synaptic activities and reducing its α7nAChR/TLR4 associations and downstream pathologies.

The interactions of TLR2, RAGE, CCR5, CXCR4 and CD4 cell surface receptors and their associated adaptor proteins such as MyD88 with FLNA are presently less well understood, but as discussed hereinafter have been found to mediate similar immune responses. As is discussed hereinafter, the immune responses mediated by one or more of TLR2, RAGE, CCR5, CXCR4 and CD4 can be inhibited by sumifilam and its related compounds.

The treatment approach disclosed below is targeted at inhibiting the immune response and thereby cytokine efflux that is mediated by the-above recited cell surface receptors. It is believed that FLNA interacts with one or more members of the intracellular signaling pathway once one or more of the TLR2, RAGE, CCR5, CXCR4 and CD4 cell surface receptors is bound by its immune response-activating ligand. That FLNA interaction causes the FLNA dimer to alter its conformation, which induces the immune response. Contacting the thus activated cells with a compound such as sumifilam as discussed herein, causes that compound to bind to FLNA, altering the FLNA configuration and thereby inhibiting that immune response and its cytokine efflux.

### BRIEF SUMMARY OF THE INVENTION

The present invention contemplates a method for inhibiting one or more of a cell surface receptor-mediated immune response, illustratively, such as inflammation of cells of the CNS. That method comprises administering an effective amount of a compound or a pharmaceutically acceptable salt thereof to mammalian cells in recognized (diagnosed) need thereof that express one or more of TLR2, RAGE, CCR5, CXR4 and CD4 cell surface receptors. The administered compound is a compound of one or more of (a) Series C-1, Formula B, (b) Series C-2, Formula I, and (c) Series D. The administration is preferably carried out in the absence of a mu opioid receptor-(MOR-)binding effective amount of a separate MOR agonist or antagonist and is often carried out a plurality of times over a period of days or months.

A compound of Series C-1, Formula B has the structural formula

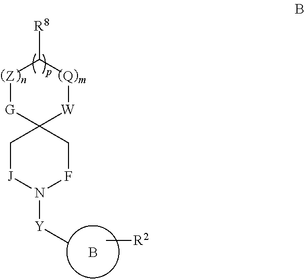

wherein

G and W are selected from the group consisting of NR²⁰, NR⁷, CH₂, and O, where R⁷ is H, C₁-C₁₂ hydrocarbyl, or C₁-C₁₂ hydrocarboyl and R²⁰ is a group X-circle A-R¹ as defined hereinafter.

X and Y are the same or different and are SO₂, C(O), CH₂, CD₂ (where D is deuterium), NHC(NH), OC(O), NHC(S) or NHC(O).

Q is CHR⁹ or C(O).

Z is CHR¹⁰ or C(O).

J and F are the same or different and are CH or CD (where D is deuterium).

Each of m, n and p is zero or one, and the sum of m+n+p is 2 or 3, preferably 2.

The circles A and B are the same or different aromatic or heteroaromatic ring systems that contain one ring or two fused rings. Groups R¹ and R² are the same or different and each is hydrogen or represents up to three substituents other than hydrogen that themselves can be the same or different, wherein each of those three groups, R^{1a-c} and R^{2a-c}, is separately selected from the group consisting of H, C₁-C₆ hydrocarbyl, C₁-C₆ hydrocarbyloxy, trifluoromethyl, trifluoromethoxy, C₁-C₇ hydrocarboyl (acyl), hydroxy-, trifluoromethyl-(—CF₃) or halogen-substituted C₁-C₇ hydrocarboyl, C₁-C₆ hydrocarbylsulfonyl, halogen, nitro, phenyl, cyano, carboxyl, C₁-C₇ hydrocarbyl carboxylate [C(O)O—C₁-C₇ hydrocarbyl], carboxamide [C(O)NR³R⁴] or sulfonamide [SO₂NR³R⁴],

wherein the amido nitrogen of either the carboxamide or sulfonamide has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, MAr, where M is —CH₂—, —O— or —N═N— and Ar is a single-ringed aryl group, and NR⁵R⁶,

wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

When present, an R⁸, R⁹, or R¹⁰ group is independently H or a C₁-C₈ hydrocarbyl group that is unsubstituted or is substituted with up to three atoms that are the same or different and are oxygen or nitrogen atoms.

A compound of Series C-2, Formula I has the structural formula

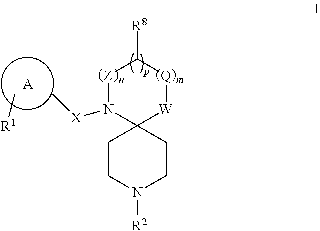

wherein

Q is CHR⁹ or C(O), Z is CHR¹⁰ or C(O), and only one of Q and Z is C(O).

Each of m and n and p is zero or one and the sum of m+n+p is 2 or 3, preferably 2.

W is NR⁷, or O, where R⁷ and R² are the same or different and are H, C(H)ᵥ(D)ₕ where each of v and h is 0, 1, 2 or 3 and v+h=3, C(H)_{q}(D)ᵣ-aliphatic C₁-C₁₁ hydrocarbyl where each of q and r is 0, 1, or 2 and q+r=0, 1 or 2, (including aliphatic C₁-C₁₂ hydrocarbyl when q+r=0), aliphatic C₁-C₁₂ hydrocarbyl sulfonyl or aliphatic C₁-C₁₂ hydrocarboyl (acyl), and X-circle A-R¹ as defined hereinafter.

J and F are the same or different and are CH₂, CHD or CD₂ (where D is deuterium).

X is SO₂, C(O) or CH₂.

Circle A is an aromatic or heteroaromatic ring system that contains a single ring or two fused rings.

R¹ is H or represents up to three substituents, R^{1a}, R^{1b}, and R^{1c}, that themselves can be the same or different, wherein each of those three groups, R^{1a-c}, is separately selected from the group consisting of H, C₁-C₆ hydrocarbyl, C₁-C₆ hydrocarbyloxy, C₁-C₆ hydrocarbyloxycarbonyl, trifluoromethyl, trifluoromethoxy, C₁-C₇ hydrocarboyl, hydroxy-, trifluoromethyl- or halogen-substituted C₁-C₇ hydrocarboyl, C₁-C₆ hydrocarbylsulfonyl, C₁-C₆ hydrocarbyloxysulfonyl, halogen, nitro, phenyl, cyano, carboxyl, C₁-C₇ hydrocarbyl carboxylate, carboxamide or sulfonamide, wherein the amido nitrogen in either amide group has the formula NR³R⁴ in which R³ and R⁴ are the same or different and are H, or C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, MAr, where M is —CH₂—, —O— or —N═N— and Ar is a single-ringed aryl or heteroaryl group and NR⁵R⁶ wherein
R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

When present, an R⁸, R⁹, or R¹⁰ group is independently H or a C₁-C₈ hydrocarbyl group that is unsubstituted or is substituted with up to three atoms that are the same or different and are oxygen or nitrogen atoms.

A compound of Series D has the structural formula

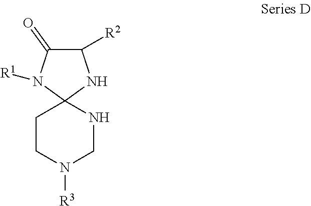

wherein

R¹ is hydrogen, a linear or branched unsubstituted or at least monosubstituted C₁₋₁₀ alkyl group that can comprise at least one heteroatom as a link; a linear or branched unsubstituted or at least monosubstituted C₂₋₁₀ alkenyl group that can comprise at least one heteroatom as a link; a linear or branched unsubstituted or at least monosubstituted C₂₋₁₀ alkynyl group that can comprise at least one heteroatom as a link; an unsubstituted or at least monosubstituted five-membered to fourteen-membered aryl group or heteroaryl group, that can be bonded via a linear or branched C₁₋₅ alkylene group that can comprise at least one heteroatom as a link; or a —C(═O)OR⁷ group that can be bonded via a linear or branched C₁₋₅ alkylene group.

R² is hydrogen, a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link, an unsubstituted or at least monosubstituted five-membered to fourteen-membered aryl or heteroaryl group, that can be bonded via a linear or branched C₁₋₅ alkylene group that can comprise at least one heteroatom as a link.

R³ is a —S(═O)₂—R⁴ group, a —C(═S)NH—R⁵ group, or a —C(═O)NH—R⁶ group.

R⁴ is an NR¹⁰R¹¹ group, a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link; an unsubstituted or at least monosubstituted five-membered to fourteen-membered aryl group or heteroaryl group, that can be bonded via a linear or branched unsubstituted or at least monosubstituted C₁₋₅ alkylene group that can comprise at least one heteroatom as a link and may be condensed with a five-membered or six-membered monocyclic ring system, an unsubstituted or at least monosubstituted C₃₋₈-cycloaliphatic group that can comprise at least one heteroatom as a ring member or that can be bonded via a linear or branched unsubstituted or at least monosubstituted C₁₋₅ alkylene group that can comprise at least one heteroatom as a link and that can be bridged by a linear or branched unsubstituted or at least monosubstituted C₁₋₅ alkylene group.

R⁵ represents a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link, an unsubstituted or at least monosubstituted five-membered to fourteen-membered aryl or heteroaryl group, that can be bonded via a linear or branched unsubstituted or at least monosubstituted C₁₋₅ alkylene group that can comprise at least one heteroatom as a link, an unsubstituted or at least monosubstituted C₃₋₈-cycloaliphatic group that can comprise at least one heteroatom as a ring member and that can be bonded via a linear or branched unsubstituted or at least monosubstituted C₁₋₅ alkylene group that can comprise at least one heteroatom as a link, a —C(═O)OR⁸ group or a —C(═O)OR⁹ group either of that can be bonded via a linear or branched C₁₋₁₀ alkylene group.

R⁶ represents an unsubstituted or at least monosubstituted five-membered to fourteen-membered aryl or heteroaryl group, which aryl or heteroaryl group may be bonded via a linear or branched unsubstituted or at least monosubstituted C₁₋₅ alkylene group that can comprise at least one heteroatom as a link, an unsubstituted or at least monosubstituted C₃₋₈-cycloaliphatic group that can comprise at least one heteroatom as a ring member, or that can be bonded via a linear or branched unsubstituted or at least monosubstituted C₁₋₅ alkylene group that can comprise at least one heteroatom as a link.

R⁷, R⁸, R⁹, R¹⁰, and R¹¹, independently represent a linear or branched C₁₋₅ alkyl group, a linear or branched C₂₋₅ alkenyl group, or a linear or branched C₂₋₅ alkynyl group.

In the description of a compound of Series D immediately above, the word “heteroatom” or the prefix “hetero” means an atom that is oxygen, nitrogen or sulfur. When a heteroatom is nitrogen, all of its valance bonds are accounted for by bonds to carbon and/or another heteroatom for a total of two heteroatoms bonded together, and within the definition of a recited R group. One bond to a nitrogen-containing substituent can also be to an acyl group containing 1-7 carbon atoms.

The use of a single stereoisomer or mixture of stereoisomers, or a pharmaceutically acceptable salt of a contemplated compound is also contemplated. The contemplated administration can take place in vivo or in vitro, and is typically repeated over a period of days or months when administered in vivo. Individual optical isomers and mixtures of optical isomers of those compounds of the above Formulas are also contemplated, as are pharmaceutically acceptable salts of those optical isomers.

A before-described a compound or a pharmaceutically acceptable salt thereof binds to filamin A or binds to a pentapeptide of filamin A as described in Example 1 hereinafter, and inhibits at least about 60 percent and more preferably at least about 70 percent of the FITC-labeled naloxone binding when present at a 10 mM concentration and using unlabeled naloxone as the control inhibitor at the same concentration as also described in Example 1 above. A contemplated compound is substantially free from binding with any other portion of FLNA at the effective concentration used.

Substantial freedom from binding with any other portion of FLNA can be determined using a titration assay such as that shown in FIG. 1A herein taken from FIG. 2 of Wang et al., PLoS One. 3(2):e1554 (2008), which in that figure indicates the presence of two binding site regions by the two inflection points shown in the plot. The presence of a single binding site is indicated by the presence of a single inflection point in a the similar of plot FIG. 1D as discussed in U.S. Pat. No. 9,354,223 using the biotinylated FLNA pentamer peptide of sequence positions 2561-2565 (FLNA pentamer) [UniProtKB/Swiss-Prot entry P21333, FLNA-HUMAN, Filamin-A protein sequence]. Substantial freedom from binding with any other portion of FLNA can also be inferred from functional data such as a cytokine release assay illustrated hereinafter that indicate contemplated compounds do not bind the second site on FLNA because the compounds are effective over a wide range of concentrations, unlike those compounds such as naloxone and naltrexone that bind to two binding sites on FLNA.

A contemplated compound described above or its pharmaceutically acceptable salt is typically administered in an effective amount dissolved or dispersed in a pharmaceutical composition. That pharmaceutical composition can be in solid or liquid form.

The invention particularly contemplates a method of inhibiting hyperinflammatory syndromes such as cytokine storm, mediated by one or more of TLR2, RAGE, CCR5, CXR4 and CD4 cell surface receptors as can sometimes occur upon infection with particularly virulent strains of influenza A, such as H1N5, RSV, SARS-CoV and SARS-CoV-2. That method is carried out by administering to a subject in diagnosed need [that presents with a severe lung infection and systemic circulatory inflammation producing systemic sepsis some of whose cells a) exhibit an inflammatory immune response such as production of inflammatory cytokines and b) contain one or more of TLR2, RAGE, CCR5, CXR4 and CD4 cell surface receptors] an effective amount of a before-described compound that binds to the FLNA pentapeptide of FLNA positions 2561-2565. The administration is carried out in the absence of a MOR-binding effective amount of a separate MOR agonist or antagonist molecule.

The present invention has several benefits and advantages.

One benefit is that a contemplated method inhibits Ab signaling through a7nAChR that is believed superior to targeting the receptor itself. Disabling the Ab-induced a7nAChR signaling without directly affecting the a7nAChRs avoids altering the sensitivity or cell surface level of the receptors, an insidious problem with using chronic receptor agonists or antagonists.

An advantage of this invention is that this approach appears to selectively affect the robust increase in filamin recruitment by Ab while preserving basal coupling, suggesting that the compounds used in the method reduce the pathological signaling by Ab, while retaining physiological a7nAChR signaling.

A further benefit of the invention is that administration of a contemplated compound or salt can provide the benefits of one or more of the methods enumerated above by binding of that compound FLNA and thereby disrupting a direct or indirect interaction of FLNA with one or more of TLR2, RAGE, CCR5, CXR4 and CD4.

Yet another advantage of the invention is that its use can inhibit or lessen the intensity of a cytokine storm that can accompany the infections and conditions noted previously by inhibiting a recipient mammal's expression of one or more of TLR2, RAGE, CCR5, CXR4 and CD4.

Still further benefits and advantages will be apparent to those skilled in the art from the disclosures that follow.

### BRIEF DESCRIPTION OF THE DRAWINGS

In the drawings forming a part of this disclosure,

FIG. 1, in four parts as FIGS. 1A, 1B, 1C and 1D, are graphs reproduced from U.S. Pat. No. 9,354,223 (FIGS. 16A-16D) that show competition curves illustrating the binding of radio-labeled naloxone [³H]NLX in the presence of naltrexone (NTX) or illustrative Compound C0105 to the filamin A (FLNA) or the FLNA pentamer peptide of FLNA positions 2561-2565 [UniProtKB/Swiss-Prot entry P21333, FLNA-HUMAN; Filamin-A protein sequence] as reported in the above U.S. Patent and in Wang et al., PLoS One. 3(2):e1554 (2008). FIG. 1A illustrates [³H]NLX binding to FLNA in the membranes of A7 cells in the presence of indicated amounts of naltrexone (NTX) and is taken from Wang et al., PLoS One. 3(2):e1554 (2008), FIG. 2; FIG. 1B illustrates binding of [³H]NLX to FLNA in the membranes of A7 cells in the presence of indicated amounts of Compound C0105; FIG. 1C illustrates binding of [³H]NLX in the presence of indicated amounts of Compound C0105 to FLNA in the membranes of SK-N-MC cells; and FIG. 1D illustrates binding of [³H]NLX to the biotinylated FLNA pentamer peptide that shows a single affinity state (FLNA positions 2561-2565) in the presence of indicated amounts of Compound C0105.

FIG. 2, in two panels as FIGS. 2A and 2B, illustrates the response of human astrocyte TLR2 receptors to simultaneous contact with Compound C0105 at 100 fM [open bars], 10 pM [diagonal line bars] and 1 nM [black bars] and with an insulting inflammation-inducing ligand [LTA-SA (lipoteichoic acid from S. aureus) and PGN-SA (peptidoglycan from S. aureus)] can substantially inhibit insult-induced release of pro-inflammatory cytokines IL-1b, IL-6 and TNFa by about 75 to about 95%. One-way ANOVA: *p<0.01 compared to vehicle-treated group for each insult.

FIG. 3, in two panels as FIGS. 3A and 3B, illustrate the FLNA-TLR2 association in cells from human postmortem frontal cortex cells via LTA-SA and PGN-SA insult-induction of the FLNA-TLR2 association, as well as the effects of 0.1 micromolar Ab₄₂ and 1 or 10 nM Compound C0105 using Western blotting (FIG. 3A) and quantitation of the proteins by densitometric analysis and comparison of ratios of TLR2/FLNA (upper portion of FIG. 3B) and the inhibition of the formation of those ratios by Compound C0105 (lower portion of FIG. 3B). One-way ANOVA: p<0.01. Newman-Keuls multiple comparisons: *p<0.01, **p<0.05 compared to vehicle baseline; #p<0.01 compared to respective ligand alone group.

FIG. 4, in three panels as FIGS. 4A, 4B and 4C, illustrates the phosphorylation of tau at each of three sites after contacting human postmortem frontal cortex cells with LTA-SA or PGN-SA, as well as the effects of 0.1 micromolar Ab₄₂ and 1 or 10 nM Compound C0105 using Western blotting (FIG. 4A), quantitation ratio of each of the individual phosphorylated tau proteins to the total amount of the blotted tau proteins by densitometric analysis (FIG. 4B), and comparison of inhibition of the phosphorylation of each site by Compound C0105 (FIG. 4C). Newman-Keuls multiple comparisons: *p<0.01 compared to vehicle baseline; #p<0.01 compared to respective ligand alone group.

FIG. 5, in four panels as FIGS. 5A, 5B, 5C and 5D, illustrates that the FLNA linkages with CXCR4, CCR5 and CD4 in the postmortem brain from an AD patient are elevated compared to those in a control postmortem brain. Thus, FIG. 5A shows western blots from brain preparations precipitated by antibodies to FLNA and visualized using antibodies to each of the indicated proteins after incubation for one hour in a vehicle containing 1 nM PTI-125 (C0105) for the control and sample from an AD patient when contacted with vehicle or the 0.1 nM PTI-125 (C0105; sumifilam) solution. The upper graphs of FIG. 5B show the ratio of CXCR4/FLNA using density data from FIG. 5A for the control versus AD in vehicle, and control versus AD in 1 nM C0105 for both the 45 KDa and the 48 KDa CXCR4 molecules, whereas the lower graphs show the percent differences over the control for each of those groups. FIG. 5C and FIG. 5D illustrate similar results to those of FIG. 5B for CD4 and CCR5, respectively.

FIG. 6 shows western blot results that illustrate that exogenous Ab₄₂ induced FLNA interactions with CXCR4, CD4 and CCR5 from lymphocytes of a young control subject (#81) matched levels in lymphocytes of an AD patient (#63). Addition of 1 nM Compound C0105 reduced these linkages in both, as did the separate addition of 1 nM naloxone (NLX).

FIG. 7, in two panels as FIGS. 7A and 7B, graphically illustrates some of the results of a Phase 2b Clinical study of the effects of Compound C0105, now known as sumifilam, on 64 mild-to-moderate AD patients, mini-mental state examination (MMSE) value of ≥16≤26, age 50-85 years, with a key inclusion at screening of CFS Total tau/ab≥0.28. The patients were divided into three groups, and each group was given one of three tablet dosages twice daily for 28 days. One patient group received a placebo, another received tablets containing 50 mg of sumifilam and the third received tablets containing 100 mg of sumifilam. Lumbar puncture screening of CFS and blood were taken prior to any administration and at day 28. The results of that screening showed that the amount of HMGB1 from the placebo increased slightly, whereas the amount from patients dosed with either dose sumifilam-containing tablets decreased significantly, p<0.001 relative to the placebo, thereby illustrating the involvement of FLNA in the release of that biomarker. FIG. 7B repeats the results shown in FIG. 7A and includes results for the additional neuroinflammation markers YKL-40 (a glycoprotein produced by inflammatory, cancer and stem cells), interleukin-6 (IL-6), soluble triggering receptor expressed on myeloid cells 2 (sTREM2), albumin (a marker for blood-brain-barrier permeability) and IgG antibodies. Stronger significance levels occurred in the 100 mg group. Data are means±SEM. *p≤0.0001, #p≤0.001, †p<0.01 and +p<0.05 versus placebo. N=22, 18 and 19 for placebo, 50 mg and 100 mg, respectively.

FIG. 8 illustrate results similar to those in FIGS. 7A and 7B, after a 6-month open-label treatment with sumifilam. Measurements of IL-6, albumin or antibodies were not taken in this study. Data are means±SD. *p≤0.00001 for baseline vs. 6 months by paired t test. N=25.

### ABBREVIATIONS AND SHORT FORMS

The following abbreviations and short forms are used in this specification.

“Ab” means amyloid-beta

“Ab_{42”} means a 42-residue proteolysis product of amyloid precursor protein (APP)

“a7nAchR” means alpha-7 nicotinic acetylcholine receptor

“CCR5” means CC chemokine receptor type 5

“CXCR-4” means an alpha-chemokine receptor specific for stromal-derived-factor-1 (SDF-1)

“CD4” means cluster of differentiation 4

“DAMGO” means [D-Ala2, N-MePhe4, Gly-ol]-enkephalin

“ERK2” means extracellular signal-regulated kinase 2

“FCX” means frontal cortex or prefrontal cortex

“FLNA” means filamin A

“FITC” means fluorescein isothiocyanate

“Gs” means G protein stimulatory subtype, stimulates adenylyl cyclase

“HP” means hippocampus

“IHC” means immunohistochemistry

“IR” means insulin receptor

“MOR” means μ opioid receptor

“NLX” means naloxone

“NTX” means naltrexone

“NFTs” means neurofibrillary tangles

“NMDA” means N-methyl-D-aspartate

“NMDAR” means NMDA receptor

“pERK2” means phosphorylated ERK2

“TLR2” means toll-like receptor-2

“TLR4” means toll-like receptor-4

### DEFINITIONS

In the context of the present invention and the associated claims, the following terms have the following meanings:

The articles “a” and “an” are used herein to refer to one or to more than one (i.e., to at least one) of the grammatical object of the article. By way of example, “an element” means one element or more than one element.

As used herein, the term “hydrocarbyl” is a short hand term for a non-aromatic group that includes straight and branched chain aliphatic as well as alicyclic groups or radicals that contain only carbon and hydrogen. Inasmuch as alicyclic groups are cyclic aliphatic groups, such substituents are deemed hereinafter to be subsumed within the aliphatic groups. Thus, alkyl, alkenyl and alkynyl groups are contemplated, whereas aromatic hydrocarbons such as phenyl and naphthyl groups, which strictly speaking are also hydrocarbyl groups, are referred to herein as aryl groups, substituents, moieties or radicals, as discussed hereinafter. An aralkyl substituent group such as benzyl is deemed an aromatic group as being an aromatic ring bonded to an X group, where X is CH₂.

A substituent group containing both an aliphatic ring and an aromatic ring portion such as tetralin (tetrahydronaphthalene) that is linked directly through the aliphatic portion to the depicted ring containing the W group is deemed a non-aromatic, hydrocarbyl group. On the other hand, a similar group bonded directly via the aromatic portion, is deemed to be a substituted aromatic group.

Where a specific aliphatic hydrocarbyl substituent group is intended, that group is recited; i.e., C₁-C₄ alkyl, methyl or dodecenyl. Exemplary hydrocarbyl groups contain a chain of 1 to about 12 carbon atoms, and preferably 1 to about 8 carbon atoms, and more preferably 1 to 6 carbon atoms.

A particularly preferred hydrocarbyl group is an alkyl group. As a consequence, a generalized, but more preferred substituent can be recited by replacing the descriptor “hydrocarbyl” with “alkyl” in any of the substituent groups enumerated herein.

Examples of alkyl radicals include methyl, ethyl, n-propyl, isopropyl, n-butyl, isobutyl, sec-butyl, tert-butyl, pentyl, iso-amyl, hexyl, octyl, decyl, dodecyl and the like. Cyclic alkyl radicals such as cyclo propyl, cyclobutyl, cyclopentyl, cyclohexyl and cycloheptyl are also contemplated, as are their corresponding alkenyl and alkynyl radicals. Examples of suitable straight and branched chain alkenyl radicals include ethenyl (vinyl), 2-propenyl, 3-propenyl, 1,4-pentadienyl, 1,4-butadienyl, 1-butenyl, 2-butenyl, 3-butenyl, decenyl and the like. Examples of straight and branched chain alkynyl radicals include ethynyl, 2-propynyl, 3-propynyl, decynyl, 1-butynyl, 2-butynyl, 3-butynyl, and the like.

Usual chemical suffix nomenclature is followed when using the word “hydrocarbyl” except that the usual practice of removing the terminal “yl” and adding an appropriate suffix is not always followed because of the possible similarity of a resulting name to one or more substituents. Thus, a hydrocarbyl ether is referred to as a “hydrocarbyloxy” group rather than a “hydrocarboxy” group as may possibly be more proper when following the usual rules of chemical nomenclature. Illustrative hydrocarbyloxy groups include methoxy, ethoxy, and cyclohexenyloxy groups. On the other hand, a hydrocarbyl group containing a —C(O)— functionality is referred to as a hydrocarboyl (acyl) and that containing a —C(O)O— is a hydrocarboyloxy group inasmuch as there is no ambiguity. Exemplary hydrocarboyl and hydrocarboyloxy groups include acyl and acyloxy groups, respectively, such as acetyl and acetoxy, acryloyl and acryloyloxy.

Carboxyl-related linking groups between the central spiro ring system and an aromatic or heteroaromatic ring system, circle A, include several types of ester and amide bonds. Illustrative of such bonds are sulfonamide, sulfonate and thiosulfonate esters that can be formed between a SO₂-containing group [also sometimes shown as a S(═O)₂ group] and an amine, oxygen or sulfur atom, respectively. Amide, ester and thioester links can be formed between an aromatic or heteroaromatic ring containing a C(O) [also sometimes shown as (C═O)] group and a nitrogen, oxygen or sulfur atom, respectively. Similarly, a guanidino linker can be formed between an aromatic or heteroaromatic ring containing a NHC(NH) [NHC(═NH)] group and a nitrogen, a urethane, carbonate or thiocarbonate can be formed between an aromatic or heteroaromatic ring containing a OC(O) [or OC(═O)] group and a nitrogen, oxygen or sulfur, respectively. A compound containing a urea linker, urethane linker or isothiourea linker [NHC(O)S] {or [NHC(═O)S]} can be formed between an aromatic or heteroaromatic ring containing a NHC(O) group and a nitrogen, oxygen or sulfur, respectively. A thiourea linkage is also contemplated.

A “carboxyl” substituent is a —C(O)OH group. A C₁-C₆ hydrocarbyl carboxylate is a C₁-C₆ hydrocarbyl ester of a carboxyl group. A carboxamide is a —C(O)NR³R⁴ substituent, where the R groups are defined elsewhere and are numbered here as 3 and 4 for ease in further discussion, but need not be so numbered in the following chemical formulas. Similarly, a sulfonamide is a —S(O)₂NR³R⁴ substituent, where the R groups are defined hereinafter. Illustrative R³ and R⁴ groups that together with the depicted nitrogen of a carboxamide form a 5-7-membered ring that optionally contains 1 or 2 additional hetero atoms that independently are nitrogen, oxygen or sulfur, include morpholinyl, piperazinyl, oxathiazolyl, 1,2,3-triazolyl, 1,2,4-triazolyl, pyrazolyl, 1,2,4-oxadiazinyl and azepinyl groups.

As a skilled worker will understand, a substituent that cannot exist such as a C₁ alkenyl or alkynyl group is not intended to be encompassed by the word “hydrocarbyl”, although such substituents with two or more carbon atoms are intended.

The term “aryl”, alone or in combination, means a phenyl, naphthyl or other radical as recited hereinafter that optionally carries one or more substituents selected from hydrocarbyl, hydrocarbyloxy, halogen, hydroxy, amino, nitro and the like, such as phenyl, p-tolyl, 4-methoxyphenyl, 4-(tert-butoxy)phenyl, 4-fluorophenyl, 4-chlorophenyl, 4-hydroxyphenyl, and the like. The term “arylhydrocarbyl”, alone or in combination, means a hydrocarbyl radical as defined above in which one hydrogen atom is replaced by an aryl radical as defined above, such as benzyl, 2-phenylethyl and the like. The term “arylhydrocarbyloxycarbonyl”, alone or in combination, means a radical of the formula —C(O)—O-arylhydrocarbyl in which the term “arylhydrocarbyl” has the significance given above. An example of an arylhydrocarbyloxycarbonyl radical is benzyloxycarbonyl. The term “aryloxy” means a radical of the formula aryl-O— in which the term aryl has the significance given above. The term “aromatic ring” in combinations such as substituted-aromatic ring sulfonamide, substituted-aromatic ring sulfinamide or substituted-aromatic ring sulfenamide means aryl or heteroaryl as defined above.

As used herein, the term “binds” refers to the specific adherence of molecules to one another, such as, but not limited to, the interaction of a ligand with its receptor, or a FLNA peptide of with a small molecule such as the compounds disclosed herein, or an antibody and its antigen.

As used herein, the term “FLNA-binding compound” refers to a compound that binds to the scaffolding protein filamin A, or more preferably to a peptide comprising residues of the FLNA sequence that correspond to amino acid residue positions 2561-2565 of the FLNA protein sequence as noted in the sequence provided at the web address: UniProtKB/Swiss-Prot entry P21333, FLNA-HUMAN, Filamin-A protein sequence. This peptide is referred to as the “5-mer FLNA peptide”, the “FLNA pentapeptide of positions 2561-2565”, the “FLNA pentapeptide of FLNA positions 2561-2565”, the “FLNA peptide” and similar names all meaning the same material. A FLNA-binding compound can inhibit the MOR-Gs coupling caused by agonist stimulation of the μ opioid receptor via interactions with filamin A, preferably in the 24^{th} repeat region.

As used herein, the term “opioid receptor” refers to a G protein-coupled receptor located in the CNS that interacts with opioids. More specifically, the μ opioid receptor is activated by opioids causing analgesia, sedation, nausea, a pro-inflammatory response and many other side effects known to one of ordinary skill in the art.

As used herein, the term “opioid agonist” refers to a substance that upon binding to an opioid receptor can stimulate the receptor, induce G protein coupling and trigger a physiological response. More specifically, an opioid agonist is a morphine-like substance that interacts with MOR to produce analgesia.

As used herein, the term “opioid antagonist” refers to a substance that upon binding to an opioid receptor inhibits the function of an opioid agonist by interfering with the binding of the opioid agonist to the receptor.

As used herein the term “ultra-low-dose” or “ultra-low amount” refers to an amount of compound that when given in combination with an opioid agonist is sufficient to enhance the analgesic potency of the opioid agonist. More specifically, the ultra-low-dose of an opioid antagonist is admixed with an opioid agonist in an amount about 1000- to about 10,000,000-fold less, and preferably about 10,000- to about 1,000,000-fold less than the amount of opioid agonist.

As used herein an “FLNA-binding effective amount” or more simply an “effective amount” refers to an amount of a contemplated compound sufficient to bind to the FLNA pentapeptide of FLNA positions 2561-2565 and perform the functions described herein, such as inhibiting a TLR2, CCR5, CXR4 and/or CD4 cell surface receptor-mediated immune response. An effective amount of a contemplated compound is most easily determined using the in vitro assay of Example 1 herein. Using that definition, an effective amount of a contemplated compound binds to a pentapeptide of FLNA positions 2561-2565, inhibits at least about 60 percent and more preferably about 70 percent of the FITC-labeled naloxone binding when present at a 10 mM concentration and using unlabeled naloxone as the control inhibitor at the same concentration and under the same conditions as the contemplated compound, and up to about twice (200 percent) the inhibition obtained with naloxone as control.

### DETAILED DESCRIPTION OF THE INVENTION

The present invention contemplates a method of inhibiting an immune response that is mediated by one or more of TLR2, RAGE, CCR5, CXR4 and CD4 cell surface receptors that comprises administering an effective amount of a compound or a pharmaceutically acceptable salt thereof to cells in recognized (diagnosed) need thereof and expressing one or more of TLR2, RAGE, CCR5, CXR4 and CD4 cell surface receptors. Illustrative of such cells are lymphocytes, cells of the CNS, epithelial cells and endothelial cells. The administered compound is a compound of one or more of (a) Series C-1, Formula B, (b) Series C-2, Formula I, and (c) Series D. The administration is preferably carried out in the absence of a mu opioid receptor-(MOR-)binding effective amount of a separate MOR agonist or antagonist and is often carried out a plurality of times over a period of days or months.

A compound of Series C-1, Formula B, has the structural formula

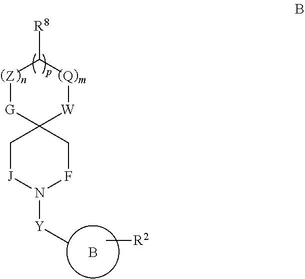

wherein

G and W are selected from the group consisting of NR²⁰, NR⁷, CH₂, and O, where R⁷ is H, C₁-C₁₂ hydrocarbyl, or C₁-C₁₂ hydrocarboyl and R²⁰ is a group X-circle A-R¹ as defined hereinafter;

X and Y are the same or different and are SO₂, C(O), CH₂, CD₂ (where D is deuterium), NHC(NH), OC(O), NHC(S) or NHC(O);

Q is CHR⁹ or C(O);

Z is CHR¹⁰ or C(O);

J and F are the same or different and are CH or CD (where D is deuterium);

each of m, n and p is zero or one and the sum of m+n+p is 2;

the circles A and B are the same or different aromatic or heteroaromatic ring systems that contain one ring or two fused rings;

groups R¹ and R² are the same or different and each is hydrogen or represents up to three substituents other than hydrogen that themselves can be the same or different, wherein each of those three groups, R^{1a-c} and R^{2a-c}, is separately selected from the group consisting of H, C₁-C₆ hydrocarbyl, C₁-C₆ hydrocarbyloxy, trifluoromethyl, trifluoromethoxy, C₁-C₇ hydrocarboyl (acyl), hydroxy-, trifluoromethyl- (—CF₃) or halogen-substituted C₁-C₇ hydrocarboyl, C₁-C₆ hydrocarbylsulfonyl, halogen, nitro, phenyl, cyano, carboxyl, C₁-C₇ hydrocarbyl carboxylate [C(O)O—C₁-C₇ hydrocarbyl], carboxamide [C(O)NR³R⁴] or sulfonamide [SO₂NR³R⁴],

wherein the amido nitrogen of either the carboxamide or sulfonamide has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, MAr, where M is —CH₂—, —O— or —N═N— and Ar is a single-ringed aryl group, and NR⁵R⁶,

wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur; and

when all of R⁸, R⁹, and R¹⁰ are present each is H, or two of R⁸, R⁹, and R¹⁰ are H and one is a C₁-C₈ hydrocarbyl group that is unsubstituted or is substituted with up to three atoms that are the same or different and are oxygen or nitrogen atoms.

A compound of Series C-2, Formula I, has the structural formula

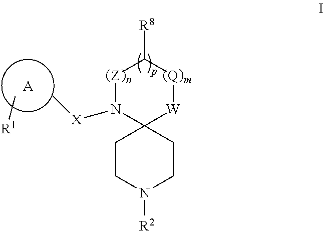

wherein

Q is CHR⁹ or C(O), Z is CHR¹⁰ or C(O), and only one of Q and Z is C(O);

each of m and n and p is zero or one and the sum of m+n+p is 2;

W is NR⁷, or O, where R⁷ and R² are the same or different and are H, C(H)ᵥ(D)ₕ where each of v and h is 0, 1, 2 or 3 and v+h=3, C(H)_{q}(D)ᵣ-aliphatic C₁-C₁₁ hydrocarbyl where each of q and r is 0, 1, or 2 and q+r=0, 1 or 2, (including aliphatic C₁-C₁₂ hydrocarbyl when q+r=0), aliphatic C₁-C₁₂ hydrocarbyl sulfonyl or aliphatic C₁-C₁₂ hydrocarboyl (acyl), and X-circle A-R¹ as defined hereinafter;

J and F are the same or different and are CH₂, CHD or CD₂ (where D is deuterium);

X is SO₂, C(O) or CH₂;

circle A is an aromatic or heteroaromatic ring system that contains a single ring or two fused rings;

R¹ is H or represents up to three substituents, R^{1a}, R^{1b}, and R^{1c}, that themselves can be the same or different, wherein each of those three groups, R^{1a-c}, is separately selected from the group consisting of H, C₁-C₆ hydrocarbyl, C₁-C₆ hydrocarbyloxy, C₁-C₆ hydrocarbyloxycarbonyl, trifluoromethyl, trifluoromethoxy, C₁-C₇ hydrocarboyl, hydroxy-, trifluoromethyl- or halogen-substituted C₁-C₇ hydrocarboyl, C₁-C₆ hydrocarbylsulfonyl, C₁-C₆ hydrocarbyloxysulfonyl, halogen, nitro, phenyl, cyano, carboxyl, C₁-C₇ hydrocarbyl carboxylate, carboxamide or sulfonamide, wherein the amido nitrogen in either amide group has the formula NR³R⁴ in which R³ and R⁴ are the same or different and are H, or C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, MAr, where M is —CH₂—, —O— or —N═N— and Ar is a single-ringed aryl or heteroaryl group and NR⁵R⁶ wherein
R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur; and

when present, R⁸ is H, or is a C₁-C₈ hydrocarbyl group that is unsubstituted or is substituted with up to three atoms that are the same or different and are oxygen or nitrogen atoms.

A compound of Series D corresponds in structure to the formula

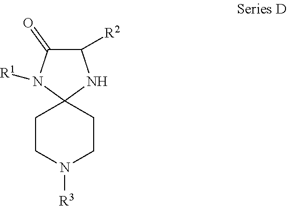

wherein

R¹ is hydrogen, a linear or branched unsubstituted or at least monosubstituted C₁₋₁₀ alkyl group that can comprise at least one heteroatom as a link; a linear or branched unsubstituted or at least monosubstituted C₂₋₁₀ alkenyl group that can comprise at least one heteroatom as a link; a linear or branched unsubstituted or at least monosubstituted C₂₋₁₀ alkynyl group that can comprise at least one heteroatom as a link; an unsubstituted or at least monosubstituted five-membered to fourteen-membered aryl group or heteroaryl group, that can be bonded via a linear or branched C₁₋₅ alkylene group that can comprise at least one heteroatom as a link; or a —C(═O)OR⁷ group that can be bonded via a linear or branched C₁₋₅ alkylene group;

R² is hydrogen, a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link, an unsubstituted or at least monosubstituted five-membered to fourteen-membered aryl or heteroaryl group, that can be bonded via a linear or branched C₁₋₅ alkylene group that can comprise at least one heteroatom as a link;

R³ is a —S(═O)₂—R⁴ group, a —C(═S)NH—R⁵ group, or a —C(═O)NH—R⁶ group;

R⁴ is an NR¹⁰R¹¹ group, a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link; an unsubstituted or at least monosubstituted five-membered to fourteen-membered aryl group or heteroaryl group, that can be bonded via a linear or branched unsubstituted or at least monosubstituted C₁₋₅ alkylene group that can comprise at least one heteroatom as a link and may be condensed with a five-membered or six-membered monocyclic ring system, an unsubstituted or at least monosubstituted C₃₋₈-cycloaliphatic group that can comprise at least one heteroatom as a ring member or that can be bonded via a linear or branched unsubstituted or at least monosubstituted C₁₋₅ alkylene group that can comprise at least one heteroatom as a link and that can be bridged by a linear or branched unsubstituted or at least monosubstituted C.sub.1-5 alkylene group;

R⁵ represents a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link, an unsubstituted or at least monosubstituted five-membered to fourteen-membered aryl or heteroaryl group, that can be bonded via a linear or branched unsubstituted or at least monosubstituted C₁₋₅ alkylene group that can comprise at least one heteroatom as a link, an unsubstituted or at least monosubstituted C₃₋₈-cycloaliphatic group that can comprise at least one heteroatom as a ring member and that can be bonded via a linear or branched unsubstituted or at least monosubstituted C₁₋₅ alkylene group that can comprise at least one heteroatom as a link, a —C(═O)OR⁸ group or a —C(═O)OR⁹ group either of that can be bonded via a linear or branched C₁₋₁₀ alkylene group;

R⁶ represents an unsubstituted or at least monosubstituted five-membered to fourteen-membered aryl or heteroaryl group, which aryl or heteroaryl group may be bonded via a linear or branched unsubstituted or at least monosubstituted C₁₋₅ alkylene group that can comprise at least one heteroatom as a link, an unsubstituted or at least monosubstituted C₃₋₈-cycloaliphatic group that can comprise at least one heteroatom as a ring member, or that can be bonded via a linear or branched unsubstituted or at least monosubstituted C₁₋₅ alkylene group that can comprise at least one heteroatom as a link; and

R⁷, R⁸, R⁹, R¹⁰, and R¹¹, independently each represent a linear or branched C₁₋₅ alkyl group, a linear or branched C₂₋₅ alkenyl group, or a linear or branched C₂₋₅ alkynyl group.

In the description immediately above of a compound of Series D, the word “heteroatom” or the prefix “hetero” means an atom that is oxygen or nitrogen. When a heteroatom is nitrogen, all of its valance bonds are accounted for by bonds to carbon and/or another heteroatom for a total of two heteroatoms bonded together, and within the definition of a recited R group. One bond to a nitrogen-containing substituent can also be to an acyl group containing 1-7 carbon atoms.

The administration is preferably carried out in the absence of a MOR-binding effective amount of a separate, exogenously provided MOR agonist or antagonist molecule. MOR-binding agonist and antagonist compounds can cause the very inflammatory response that the present invention inhibits. Thus, an exogenously supplied MOR-binding compound such as morphine, codeine, oxycodone and the like MOR-binding compounds is preferably absent when a contemplated compound is administered to the cells. The presence of an endogenously supplied MOR-binding compound such as an endorphin or an enkephalin cannot be as readily controlled and is not excluded. Some of the contemplated FLNA pentapeptide-binding compounds also bind to MOR and their use is also not excluded.

However, it is preferred to use a compound that binds poorly if at all to MOR, as discussed hereinafter, and is not a MOR agonist. Such a compound exhibits less than about 80 percent the MOR stimulation provided by DAMGO at the same concentration and assay conditions.

TLR2-mediated inflammation can both be recognized by the greater than background abundance of TLR2 receptors and their activation protein markers such as the cytokines IL-1b, IL-6 and TNFa that are typically enhanced together, and/or the separately stimulated NF-kB and JNK proteins. Enhanced expression of IL-1b, IL-6 and TNFa as compared to expression of NF-kB and JNK appear to proceed by different TLR2-mediated pathways. Both sets of cytokines can sometimes be present at the same time due to the same immunostimulus.

Thus, the presence of an enhanced amount of one, two or three of IL-1b, IL-6 and TNFa relative to the amount present in a non-inflammatory condition indicates the presence of TLR2-mediated inflammation. Similarly, the enhanced presence of the transcription factor NF-kB and the mitogen-activated protein kinase c-Jun N-terminal kinase (JNK) compared to the amount present in a non-inflammatory condition separately implies the presence of TLR2-mediated inflammation.

Administration of a contemplated compound or its pharmaceutically acceptable salt is typically continued until the amount of the TLR2 activation protein markers are at or near background levels as is discussed hereinafter. Enhancement of the level of TLR2 protein markers relative to background (in the absence of a TLR2-mediated immune response) condition is determined by a difference that is statistically significant at least at the 90 percent confidence level (p<0.1), and preferably at the 95 percent confidence level (p<0.05) as are illustrated in the accompanying drawings.

It is also preferred that an administered compound or a pharmaceutically acceptable salt thereof be present dissolved or dispersed in a pharmaceutically acceptable diluent as a pharmaceutical composition when administered. Most preferably, the administration is peroral.

The use of a pharmaceutically acceptable salt of a contemplated compound is also contemplated, as is the use of a single stereoisomer or mixture of stereoisomers, or of their pharmaceutically acceptable salts. The contemplated administration can take place in vivo or in vitro.

These cytokines can be assayed in lysates of cultured cells such as lymphocytes such as B cells, T cells and macrophages, epithelial cells and endothelial cells such as olfactory neurons that can be obtained by scraping the nasal cavity for neural epithelial cells for in vivo assays. The proteins can also be assayed in the cell culture medium for in vitro studies using lymphocytes or CNS cells, epithelial cells and endothelial cells such as those illustrated hereinafter and in body fluids such as cerebral spinal fluid (CSF), blood or its constituent plasma or serum or lymphocytes for in vivo assays.

It is thus to be understood that TLR2-TLR1, TLR2-TLR6 dimer-mediated and TLR4/TLR4 dimer-mediated inflammation can induce the production some of the same cytokines and chemokines. However, their pathways of intermediate signalling enzymes differ.

Thus, both TLR2- and TLR4-containing receptors utilize the CD14 (cluster of differentiation-14) receptor to bind to an insulting ligand that is the cause of the inflammation and MyD88 (myeloid differentiation primary-response protein-88) adaptor family members, including MyD88, and TIRAP (TIR domain-containing adapter protein). TLR4 utilizes TRIF, TRAM and BTK (Bruton agammaglobulinemia tyrosine kinase) in signaling, whereas TLR2 does not. TLR2/TLR1 and TLR2/TLR6 dimers also signal via through MyD88 and TIRAP, but thereafter utilize PI3K, RIP2 and Rac.

Both pathways proceed via the MyD88 adaptor protein to link the TLR receptors to the IRAK1 (interleukin-1 receptor-associated kinase-1) and IRAK4 serine/threonine kinases, leading to a MyD88-dependent pathway. Thus, upon activation of all three pairs of TLR2- and 4-containing dimers, MyD88 recruits IRAK4, thereby allowing the association of IRAK1. IRAK4 then induces the phosphorylation of IRAK1 and the similarity of produced inflammatory cytokines and chemokines.

A distinguishing feature between TLR4-mediated inflammatory responses and those that are TLR2-mediated is the enhanced presence of one or more of TLR2, PI3K, RIP2 and/or Rac or an unique polynucleotide that encodes an about 10 to about 20 amino acid residue polypeptide sequence that is unique to one of those proteins, or a plurality of such polynucleotides and polypeptides, each of which is unique to one or more of TLR2, PI3K, RIP2 and Rac.

Thus, illustratively, antibodies that immunoreact with RIP2 are commercially available from abcam (Cat. No. ab8427; Cambridge, Mass.), Invitrogen, (Cat. No. PA5-14954; Waltham, Mass.), whereas antibodies that immunoreact with RAC1 are available from Invitrogen (Cat. No. PA1-091) and from Santa Cruz Biotechnology (Cat. No. sc-514583; Dallas, Tex.), and antibodies that immunoreact with PI3K are available from Rockland Antibodies and Assays (Cat. No. 100-401-862; Limerick, Pa.). Antibodies that immunoreact with TLR2 are available from Aviva Systems Biology (Cat. No. OAPA00318; San Diego, Ca.), Invitrogen (Cat. No. 11-9021-82) and several further commercial supplies. Antibodies that immunoreact with TLR4 are similarly available from Invitrogen (Cat. No. 14-9917-82; No. 48-2300; and several others), and Santa Cruz Biotechnology (Cat. No. sc-293072).

The amino acid residue sequences and DNA sequences of TLR2, PI3K, RIP2 and Rac are known and are reported in the UniProtKB/Swiss-Prot data base system. Thus, human TLR2 is catalogued as entry No. 060603; human PI3K in its four parts is catalogued as entry No. P27986 for PIK3R1, entry No. P48736 for PIK3CG, entry No. Q8NEB9 for PIK3C3, and entry No. O00750 for PIK3C2B, as entry No. O43353 for human RIP2; and as entry No. P31749 for human Rac. A skilled worker can readily prepare his or her own nucleic acid binding probe unique to each of TLR2, PI3K, RIP2 and Rac using the sequence data provided by the UniProtKB/Swiss-Prot data base.

These proteins, polypeptides and polynucleotides can be assayed in lysates of cultured cells such as lymphocytes such as B cells, T cells and macrophages or CNS cells such as olfactory neurons that can be obtained by scraping the nasal cavity for neural epithelial cells for in vivo assays. The proteins and nucleic acids can also be assayed in the cell culture medium for in vitro studies using lymphocytes or CNS cells such as those illustrated hereinafter and in body fluids such as blood or its constituent plasma or serum or lymphocytes for in vivo assays.

Administration of a contemplated compound or its pharmaceutically acceptable salt is typically continued until the amount of one or more of the TLR2, PI3K, RIP2 and Rac activation protein markers is within about 15 percent, more preferably about 10 percent, and most preferably about 5 percent of background levels. Enhancement of the level of one or more of the TLR2 protein markers relative to background (in the absence of a TLR2-mediated immune response) condition is determined by a difference that is at least 1 standard deviation from the mean amount of the normal (well) population and preferably at least two standard deviations from that mean, and most preferably three standard deviations or more from the mean value. It is understood that a difference of 3 standard deviations is equivalent to a 99.9 percent confidence level, with greater differences being generally without meaning at least in this situation.

Determination that an inflammatory condition is mediated by one or more of CCR5, CXR4 and CD4, in addition to or separate from TLR2, is more straight forward. In those cases, antibody and/or polynucleotide assays can be used to determine whether or not one or more of those proteins is present in an amount that is that is at least 1 standard deviation greater than the mean amount of the normal (well subject) population.

For example, anti-CCR5 and anti-CXCR4 antibodies can obtained from Invitrogen, whose product catalogue lists 27 antibody preparations that react with CCR5, and 56 antibody preparations that immunoreact with CXCR4. BD Biosciences (Franklin Lakes, N.J.) lists one IgG monoclonal to CD4 (RPA4). The UniProtKB/Swiss-Prot data base lists CCR5 as entry P51681; human CXCR is listed there under the entry P61073; and human CD4 is listed under the entry P01730.

It is also preferred that an administered compound or a pharmaceutically acceptable salt thereof be present dissolved or dispersed in a pharmaceutically acceptable diluent as a pharmaceutical composition when administered. Most preferably, the administration is peroral.

The use of a pharmaceutically acceptable salt of a contemplated compound is also contemplated, as is the use of a single stereoisomer or mixture of stereoisomers, or of their pharmaceutically acceptable salts. The contemplated administration can take place in vivo or in vitro.

In presently preferred embodiments, the present invention contemplates a method of inhibiting an immune response (e.g., inflammation) mediated by one or more of TLR2, RAGE, CCR5, CXR4 and CD4 cell surface receptors in lymphocytes, cells of the CNS, epithelial cells and endothelial cells that comprises administering to those cells in recognized need thereof an effective amount of a compound of one or more of Series C-1, Series C-2, and Series D single enantiomer, a mixture of enantiomers or a pharmaceutically acceptable salt of any contemplated compound(s). The administration is preferably carried out in the absence of a MOR-binding effective amount of a separate MOR agonist or antagonist molecule.

Illustrative of CNS cells are cells such as those of a host mammal that exhibit inflammation induced by brain injury such as traumatic brain injury, chronic traumatic encephalopathy, as well as those of a host animal such as a human exhibiting Alzheimer's disease (AD) symptoms, frontotemporal dementia (FTD), progressive supranuclear palsy, dementia pugilistica and corticobasal degeneration, as well as infection by Gram positive and/or Gram negative bacteria, as well as infection caused by virus such a influenza A, SARS, MERS, and SARS-CoV-2. Illustrative lymphocytes include leukocytes such as B cells, T cells, monocytes, macrophages, eosinophils and splenocytes. Exemplary epithelial cells include those of the mucosa such as cells of the oral cavity, the ear canal and eye, the airways, the gut, and the reproductive tract. Illustrative endothelial cells include cells that line the walls of blood vessels, human aortic endothelial cells (HAEC), human pooled umbilical endothelial cells (HUVEC), human lung microvascular endothelial cells, and human coronary artery endothelial cells (HCAEC) and the like, as well as endocardial cells that are noted to be similar embryologically and biologically to endothelial cells and are included with endothelial cells.

In accordance with a method described above, a composition that contains an effective amount of a contemplated compound or its pharmaceutically acceptable salt dissolved or dispersed in a pharmaceutically acceptable diluent is administered to cells in recognized need thereof, in vivo in a living animal or in vitro in a cell preparation. When administered in vivo to an animal such as a laboratory rat or mouse or a human in recognized need, the administration inhibits an immune response (e.g., inflammation) mediated by one or more of TLR2, RAGE, CCR5, CXR4 and CD4. Admixture of a composition containing an effective amount of a contemplated compound or its pharmaceutically acceptable salt dissolved or dispersed in a pharmaceutically acceptable diluent with cells such as those discussed above in vitro also inhibits an immune response (e.g., inflammation) mediated by one or more of TLR2, RAGE, CCR5, CXR4 and CD4 as is illustrated hereinafter.

A contemplated compound binds to the scaffolding FLNA protein, and particularly to a five-residue portion of the FLNA protein sequence of positions 2561-2565 in an in vitro assay that is discussed hereinafter in Example 1, and briefly below. A contemplated compound binds only to a single site on FLNA and that site is the FLNA pentapeptide site.

Binding studies of the naltrexone inhibition of tritiated-naloxone, [³H]NLX, binding to membranes from FLNA-expressing A7 cells (an astrocyte cell line produced by immortalizing optic nerve astrocytes from the embryonic Sprague-Dawley rat with SV40 large T antigen) has shown the existence of two affinity sites on FLNA; a high affinity site (H) with an IC₅₀-H of 3.94 picomolar and a lower affinity site (L) IC₅₀-L of 834 picomolar. [Wang et al., PLoS One. 3(2):e1554 (2008); Wang et al., PLoS One. 4(1):e4282 (2009).] The high affinity site was subsequently identified as the FLNA pentapeptide of FLNA positions 2561-2565 (U.S. Pat. No. 8,722,851), whereas the lower affinity site has not yet been identified.

Compounds such as naloxone (NLX), naltrexone (NTX), methadone, fentanyl, nalorphine, nalbuphine and buprenorphine, and the like bind well to the high affinity FLNA pentapeptide of FLNA positions 2561-2565. However, when used at a dosage recited on the product label, those compounds also bind to the lower affinity site on FLNA, and typically also bind to the MOR. Some of the compounds are MOR antagonists such as naloxone, naltrexone, nalbuphine, whereas others such as methadone, buprenorphine and fentanyl are full or partial agonists of MOR. Binding to that lower affinity FLNA site impairs the activity of the FLNA pentapeptide of FLNA positions 2561-2565 to exhibit its activities as discussed, utilized and illustrated herein. Consequently, compounds such as naloxone, naltrexone, methadone, fentanyl, nalorphine, nalbuphine, buprenorphine and similar compounds that also bind to the lower affinity site on the FLNA protein are not contemplated for use herein.

A compound contemplated for use in the present invention inhibits the binding of fluorescein isothiocyanate-labeled naloxone (FITC-NLX) to biotin-linked FLNA pentapeptide of positions 2561-2565 bound to coated streptavidin plates under conditions discussed in Example 1 herein to an extent that is at least about 60 percent and more preferably at least about 70 percent of the value obtained of the value obtained when present at a 10 mM concentration and using naloxone as the control inhibitor at the same concentration as the contemplated compound, and up to about twice the value obtained with naloxone as control.

Naltrexone (NTX) can also be used as a control inhibitor. Average inhibition values obtained using NTX rather than NLX tend to be 1 or 2 percent lower in absolute value than those obtained with NLX. Thus, for example, where an average inhibition value at a particular concentration of NLX is 40 percent, one can expect values obtained with NTX to be about 38 or 39 percent. The binding inhibition values for a contemplated compound are determined taking the expected NLX/NTX value difference into account.

Representative compounds from the present Series C-1 and Series C-2 groups were examined by Ricerca Biosciences LLC of Taipei, Taiwan, in competitive binding assay studies using published techniques to determine whether the compounds could competitively inhibit binding to any of more than 65 receptors, channels and transporters including adrenergic receptors to which noradrenalin binds, serotonin receptors, muscarinic receptors to which BTX binds and cannabinoid receptors. The studied compounds each exhibited no significant inhibition in each of those assays.

### Specifically Contemplated FLNA-Binding Compounds

A compound contemplated for use in a contemplated method binds to the FLNA pentapeptide of positions 2561-2565. Such a compound can have a varied structure as noted before. Regardless of that structural variance, a contemplated compound inhibits the binding of labeled naloxone (FITC-NLX) to the biotinylated FLNA pentapeptide of positions 2561-2565 bound to coated streptavidin plates to an extent that is at least about 70 percent of the value obtained when using naloxone as an inhibitor at the same concentration and under conditions discussed hereinafter in Example 1, and can be about twice the value for naloxone at the same concentration.

Compounds having three exemplary structures have been found to bind well to the pentapeptide of FLNA positions 2561-2565. Those compounds are referred to herein as Series C-1, Series C-2, and Series D.

A pharmaceutically acceptable salt of a compound of each of the above Formulas is also contemplated. A compound having an asymmetrical (chiral) carbon or a salt of such a compound can exist in the form of stereoisomers, that are two enantiomers. The invention relates both to each enantiomer separately, and to their mixture; i.e., to both enantiomeric forms (d and l, or R and S) and to their mixture. Additionally, where two or more chiral centers are present, stereoisomers called diastereomers can form, and diastereomers are also contemplated.

As will be seen from the following definitions, a contemplated compound can contain one or more deuterated carbon atoms, in which deuterium is designated by its usual chemical designation, D. Deuterated compounds can be useful in studying the mechanism of drug interactions with living organisms for the elucidation of metabolic and biosynthetic pathways. Deuteration can also extend the half-life of a contemplated compound in vivo because a carbon-deuterium (C-D) bond is stronger than a Carbon-hydrogen (C—H) bond thereby requiring more energy input for bond cleavage. See, Blake et al., 1975 J. Pharm. Sci. 64(3):367-391; and Nelson et al., 2003 Drug Metab. Dispos. 31(12):1481-1498, and the citations therein. Contemplated deuterated compounds are prepared using well-known reactions.

A compound of Series C-1 corresponds generally to the Formula B, below

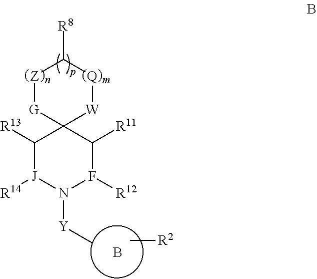

In Formula Series C-1 Formula B, G and W are selected from the group consisting of NR²⁰, NR⁷, CH₂, and O, where R⁷ is H, C₁-C₁₂ hydrocarbyl, or C₁-C₁₂ hydrocarboyl (acyl) and R²⁰ is a group X-circle A-R¹ as defined hereinafter, and G and W are preferably NR²⁰ and NR⁷. In one preferred embodiment, only one of G and W is NR⁷ and one of G and W must be NR⁷ or NR²⁰.

X and Y are the same or different and are SO₂, C(O), CH₂, CD₂ (where D is deuterium), OC(O), NHC(NH), NHC(S) or NHC(O).

Q is CHR⁹ or C(O); Z is CHR¹⁰ or C(O). J and F are the same or different and are CH or CD (where D is deuterium).

Each of m, n and p is zero or one and the sum of m+n+p is 2 or 3 for all embodiments. Each of m, and n is preferably 1, and p is preferably zero so that the sum of m+n+p is preferably 2.

The circles A and B are the same or different aromatic or heteroaromatic ring systems. Groups R¹ and R² are the same or different and each can be hydrogen or represent up to three substituents other than hydrogen that themselves can be the same or different; i.e., R^{1a}, R^{1b}, and R^{1c}, and R^{2a}, R^{2b}, and R^{2c}. Each of those six groups, R^{1a-c} and R^{2a-c}, is separately selected from the group consisting of H, C₁-C₆ hydrocarbyl, C₁-C₆ hydrocarbyloxy, C₁-C₆ hydrocarbyloxycarbonyl, trifluoromethyl, trifluoromethoxy, C₁-C₇ hydrocarboyl (acyl), hydroxy-, trifluoromethyl- (—CF₃) or halogen-substituted C₁-C₇ hydrocarboyl, C₁-C₆ hydrocarbylsulfonyl, C₁-C₆ hydrocarbyloxysulfonyl, halogen, nitro, phenyl, cyano, carboxyl, C₁-C₇ hydrocarbyl carboxylate [C(O)O—C₁-C₇ hydrocarbyl], carboxamide [C(O)NR³R⁴] or sulfonamide [S(O)₂NR³R⁴] wherein the amido nitrogen in either group has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur,

MAr, where M is —CH₂—, —O— or —N═N— and Ar is a single-ringed aryl group as described previously, and NR⁵R⁶, wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

R⁸, R⁹, and R¹⁰ are each H, or two of R⁸, R⁹, and R¹⁰ are H and one is a C₁-C₈ hydrocarbyl group that is unsubstituted or is substituted with up to three atoms that are the same or different and are oxygen or nitrogen atoms.

R¹¹, R¹², R¹³ and R¹⁴ are all H, or one of the pair R¹¹ and R¹² or the pair R¹³ and R¹⁴ together with the depicted ring form a saturated or unsaturated 6-membered ring, and the other pair are each H, or they are H and D as recited herein (in this subparagraph).

Also, in the above preferred embodiment, R¹ and R² are not both methoxy when X and Y are both SO₂, W is O and p is zero.

In another preferred embodiment,

i) only one of G and W is NR²⁰,

ii) one of G and W must be NR²⁰,

iii) one of G and W is other than NR⁷ in which R⁷ is H or an aliphatic C₁ hydrocarbyl; i.e., methyl, when (a) the sum of m+n+p is 2, and (b) the other of G and W is NR²⁰ bonded to a Z or Q, respectively, that is C(O).

R¹ and R² are preferably also not both methoxy when X and Y are both SO₂, W is O and p is zero in the above-preferred embodiment.

A pharmaceutically acceptable salt of a compound of Series C-1 Formula B and all of the remaining Series C-1 formulas disclosed herein is also contemplated.

In all of the following sub-generic formulas of a compound of Series C-1, the formula letters of G, J, F, W, Q, Z, n, m, p, X, Y, circle A and circle B and all R groups are as previously defined for a compound of Formula B of Series C-1, unless otherwise defined. Additionally, the previously stated preferences also apply unless a depicted structural formula precludes such a preference.

More preferably, a compound of Series C-1 Formula B corresponds in structure to Series C-1 Formula I, below

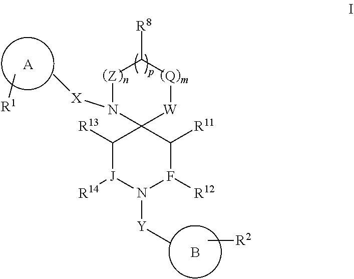

In Series C-1 Formula I, X and Y are the same or different and are SO₂, C(O), CH₂, CD₂, NHC(NH), OC(O), NHC(S) or NHC(O).

W is NR⁷, CH₂, or O, where R⁷ is H, C₁-C₁₂ hydrocarbyl, or C₁-C₁₂ hydrocarboyl (acyl), and is preferably NR⁷.

Q is CHR⁹ or C(O); and Z is CHR¹⁰ or C(O).

J and F are the same or different and are CH or CD (where D is deuterium).

each of m, n and p is zero or one and the sum of m+n+p is 2 or 3, preferably 2; and

the circles A and B are the same or different aromatic or heteroaromatic ring systems that contain one ring or two fused rings. Groups R¹ and R² are the same or different and each can be hydrogen or represent up to three substituents other than hydrogen that themselves can be the same or different; i.e., R^{1a}, R^{1b}, and R^{1c}, and R^{2a}, R^{2b}, and R^{2c}. Each of those six groups, R^{1a-c} and R^{2a-c}, is separately selected from the group consisting of H, C₁-C₆ hydrocarbyl, C₁-C₆ hydrocarbyloxy, trifluoromethyl, trifluoromethoxy, C₁-C₇ hydrocarboyl (acyl), hydroxy-, trifluoromethyl- (—CF₃) or halogen-substituted C₁-C₇ hydrocarboyl, C₁-C₆ hydrocarbylsulfonyl, halogen (F, Cl or Br, and preferably Cl), nitro, phenyl, cyano, carboxyl, C₁-C₇ hydrocarbyl carboxylate [C(O)O—C₁-C₇ hydrocarbyl], carboxamide [C(O)NR³R⁴] or sulfonamide [SO₂NR³R⁴] wherein the amido nitrogen of either group (the carboxamide or sulfonamide) has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, MAr, where M is where M is —CH₂—, —O— or —N═N— and Ar is a single-ringed aryl group, and NR⁵R⁶
wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

R⁸, R⁹, and R¹⁰ are each H, or two of R⁸, R⁹, and R¹⁰ are H and one is a C₁-C₈ hydrocarbyl group that is unsubstituted or is substituted with up to three atoms that are the same or different and are oxygen or nitrogen atoms; and

R¹¹, R¹², R¹³ and R¹⁴ are all H, or R¹¹ and R¹³ are H and R¹² and R¹⁴ are H or D, or one of the pair R¹¹ and R¹² or the pair R¹³ and R¹⁴ together with the depicted ring form a saturated or unsaturated 6-membered ring, and the other pair are each H or they are H and D as recited herein (in this subparagraph).

In some preferred embodiments, X and Y are the same. X and Y are preferably both C(O) or both SO₂, and more preferably are both SO₂. In those and other embodiments, W is preferably O. It is also preferred that p be zero.

A contemplated aromatic or heteroaromatic ring system of circle A or circle B can contain one ring or two fused rings, and preferably contains a single aromatic ring. An illustrative aromatic or heteroaromatic ring system is selected from the group consisting of phenyl, pyridyl, pyrazinyl, pyrimidinyl, pyridazinyl, triazinyl (1,3,5-triazinyl, 1,2,4-triazinyl and 1,2,3-triazinyl), furanyl, thienyl, oxazolyl, isoxazolyl, thiazolyl, isothiazolyl, naphthyl, benzofuranyl, isobenzofuranyl, benzothiophenyl, isobenzothiophenyl, benzoxazolyl, benzisoxazole, quinolyl, isoquinolyl, quinazolyl, cinnolinyl, quinoxalinyl, naphthyridinyl, benzopyrimidinyl, and mixtures thereof. The mixtures of the previous sentence occur when circle A and circle B aromatic or heteroaromatic ring systems are different.

An illustrative single-ringed aryl group of substituent MAr is selected from the group consisting of phenyl, pyridyl, pyrazinyl, pyrimidinyl, pyridazinyl, triazinyl (1,3,5-triazinyl, 1,2,4-triazinyl and 1,2,3-triazinyl), furanyl, thienyl, oxazolyl, isoxazolyl, thiazolyl and isothiazolyl.

Phenyl is a preferred aromatic or heteroaromatic ring system of circle A and circle B. Phenyl, pyridinyl and furanyl are preferred single-ringed aryl groups, Ar, of a MAr substituent, with phenyl being particularly preferred.

There are several independent and separate preferences regarding the substituent R groups. Thus, R¹ and R² are preferably the same single substituent other than hydrogen, so that circle A and circle B both contain a single substituent other than hydrogen. The single substituent of R¹ and R² is preferably located at the same relative position in their respective ring systems.

Thus, X and Y can form a sulfonamido, a carboxamido, a urea, a thiourea, a guanidino or methylene linkage from the circle A or circle B ring system to a depicted nitrogen atom of the central spiro ring. A compound having a central ring that is a spiro 6,6-ring system or a spiro 5,6-ring system, along with one nitrogen and one oxygen or two nitrogen atoms is contemplated.

In preferred practice, p is zero, and R¹¹, R¹⁴, R¹² and R¹³ are all H, so the central ring is a spiro 5,6-ring system whose 6-membered ring is unsubstituted and in which the spiro bonds are in the 4-position relative to the nitrogen of the 6-membered ring. It is separately preferred that W be O. A compound in which X and Y are the same is preferred. It is also separately preferred that X and Y both be SO₂ (sulfonyl).

A particularly preferred compound of Series C-1 Formula B that embodies the above separate preferences is a compound of Series C-1 Formula II

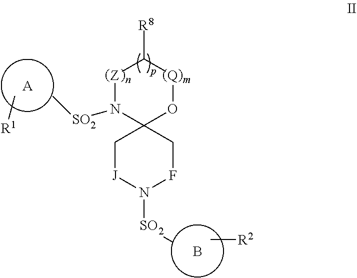

wherein

circle A and circle B, Z, Q, m, n, p, R¹, R² and R⁸ are as described above for a compound of Series C-1, unless the formula as shown precludes a definition provided for a compound of Formula B; and J and F are the same or different and are CH₂, CHD or CD₂ (where D is deuterium).

It is more preferred that circle A and circle B are each phenyl, furanyl or pyridyl and R¹ and R² is each a single substituent. There are several independent and separate preferences regarding the substituent R groups. Thus, R¹ and R² are preferably the same. R¹ and R² are also preferably located at the same relative position in their respective rings. Thus, if R¹ is 4-cyano, R² is also 4-cyano. It is also preferred that the sum of m+n+p=2 so that the upper depicted ring contains 5-ring atoms.

Preferred R¹ and R² substituent groups do not themselves provide a positive or negative charge to a compound in an aqueous medium at a pH value of about 7.2-7.4.

In other embodiments, a particularly preferred compound of Series C-1 Formula B is a compound of Series C-1 Formula III

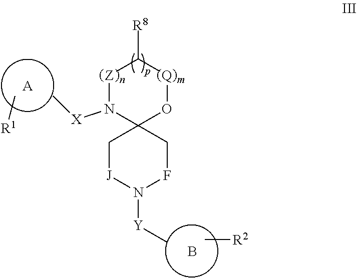

wherein

circle A and circle B, Z, Q, m, n, p, R¹, R² and R⁸ are as described previously for a compound of Series C-1 unless the formula as shown precludes a prior definition; J and F are the same or different and are CH₂, CHD or CD₂ (where D is deuterium); and X and Y are both CO, or X and Y are different and are SO₂, C(O), CH₂, CD₂ (where D is deuterium), OC(O), NHC(NH), NHC(S) or NHC(O). Previous preferences are also applicable unless precluded by the above structural formula.

More preferably, circle A and circle B are each phenyl, furanyl or pyridyl. R¹ and R² are the same and are selected from the group consisting of trifluoromethyl, C₁-C₆ acyl, C₁-C₄ alkylsulfonyl, halogen, nitro, cyano, carboxyl, C₁-C₄ alkyl carboxylate, carboxamide wherein the amido nitrogen has the formula NR³R⁴ wherein R³ and R⁴ are the same or different and are H, C₁-C₄ alkyl, and NR⁵R⁶ wherein R⁵ and R⁶ are the same or different and are H, C₁-C₄ alkyl, C₁-C₄ acyl, C₁-C₄ alkylsulfonyl.

It is still more preferred that R¹ and R² each be a single substituent. There are several independent and separate preferences regarding the substituent R groups. R¹ and R² are preferably the same. R¹ and R² are also preferably located at the same relative position in their respective rings. Thus, if R¹ is 4-cyano, R² is also 4-cyano. It is also preferred that p=0, and that the sum of m+n+p=2, so that the upper depicted ring contains 5-ring atoms.

In still further embodiments, a particularly preferred compound of Series C-1 Formula B is a compound of Series C-1 Formula IV

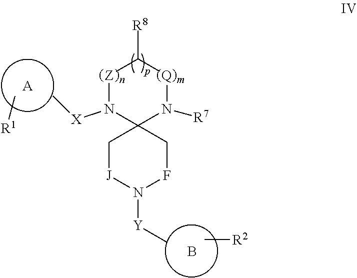

wherein

circle A and circle B, Z, Q, m, n, p, R¹, R², R⁷ and R⁸ are as described previously for a compound of Series C-1 unless the formula as shown precludes such a prior definition; J and F are the same or different and are CH₂, CHD or CD₂ (where D is deuterium); and X and Y are the same or different and are SO₂, C(O), CH₂, CD₂ (where D is deuterium), OC(O), NHC(NH), NHC(S) or NHC(O). Previous preferences are also applicable unless precluded by the above structural formula.

More preferably, circle A and circle B are each phenyl, furanyl or pyridyl. R¹ and R² are the same and are selected from the group consisting of trifluoromethyl, C₁-C₆ acyl, C₁-C₄ alkylsulfonyl, halogen, nitro, cyano, carboxyl, C₁-C₄ alkyl carboxylate, carboxamide wherein the amido nitrogen has the formula NR³R⁴ wherein R³ and R⁴ are the same or different and are H, C₁-C₄ alkyl, and NR⁵R⁶ wherein R⁵ and R⁶ are the same or different and are H, C₁-C₄ alkyl, C₁-C₄ acyl, C₁-C₄ alkylsulfonyl.

It is still more preferred that R¹ and R² each be a single substituent. There are several independent and separate preferences regarding the substituent R groups. R¹ and R² are preferably the same. R¹ and R² are also preferably located at the same relative position in their respective rings. Thus, if R¹ is 4-cyano, R² is also 4-cyano. It is also preferred that the sum of m+n+p=2, so that the upper depicted ring contains 5-ring atoms.

It is noted that the previously mentioned preferences regarding J, F, G, Q, W, X, Y, Z, n, m, p, circle A and circle B, and all of the R groups as are appropriate for a particular formula apply to a compound of Series C-1 Formulas B, and I-IV.

A compound of Series C-2 corresponds structurally to Formula I, below

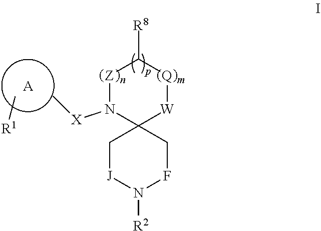

In Series C-2 Formula I,

Q is CHR⁹ or C(O), Z is CHR¹⁰ or C(O), and only one of Q and Z is C(O).

each of m and n and p is zero or one and the sum of m+n+p is 2 or 3, preferably 2;

W is NR⁷ or O, where R⁷ and R² are the same or different and are H, C(H)ᵥ(D)ₕ where each of v and h is 0, 1, 2 or 3 and v+h=3, C(H)_{q}(D)ᵣ-aliphatic C₁-C₁₁ hydrocarbyl where each of q and r is 0, 1, or 2 and q+r=0, 1 or 2, (including aliphatic C₁-C₁₂ hydrocarbyl when q+r=0), aliphatic C₁-C₁₂ hydrocarbyl sulfonyl or aliphatic C₁-C₁₂ hydrocarboyl (acyl).

Preferably, in one embodiment,

J and F are the same or different and are CH or CD (where D is deuterium);

X is SO₂, C(O), CH₂, CD₂, OC(O), NHC(NH), NHC(S) or NHC(O), preferably SO₂, C(O) or CH₂. In some embodiments, X is more preferably CH₂ or SO₂. In other embodiments, X is preferably SO₂, NHC(NH), NHC(S) or NHC(O).

Circle A is an aromatic or heteroaromatic ring system that preferably contains a single ring, but can also contain two fused rings. R¹ is H or represents up to three substituents, R^{1a}, R^{1b}, and R^{1c}, that themselves can be the same or different, wherein each of those three groups, R^{1a-c}, is separately selected from the group consisting of H, C₁-C₆ hydrocarbyl, C₁-C₆ hydrocarbyloxy, C₁-C₆ hydrocarbyloxycarbonyl, trifluoromethyl, trifluoromethoxy, C₁-C₇ hydrocarboyl, hydroxy-, trifluoromethyl- (—CF₃) or halogen-substituted C₁-C₇ hydrocarboyl, C₁-C₆ hydrocarbylsulfonyl, C₁-C₆ hydrocarbyloxysulfonyl, halogen (F, Cl, or Br, and preferably Cl) nitro, phenyl, cyano, carboxyl, C₁-C₇ hydrocarbyl carboxylate [C(O)O—C₁-C₇ hydrocarbyl], carboxamide [C(O)NR³R⁴] or sulfonamide [S(O)₂NR³R⁴],
wherein the amido nitrogen in either amide group has the formula NR³R⁴ in which R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur,

MAr, where M is —CH₂—, —O— or —N═N— and Ar is a single-ringed aryl or heteroaryl group and NR⁵R⁶ wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

R⁸, R⁹, and R¹⁰ are each H, which is preferred, or two of R⁸, R⁹, and R¹⁰ are H and one is a C₁-C₈ hydrocarbyl group that is unsubstituted or is substituted with up to three atoms that are the same or different and are oxygen or nitrogen atoms (including hydrogens as appropriate).

In a preferred embodiment of a compound of Formula I, above,

Q is CHR⁹ or C(O); and

Z is CHR¹⁰ or C(O), with the other of J, F, X, Z, n, m, circle A, all of the R groups being defined as discussed above unless precluded by the structural formula, and p=zero (0).

A pharmaceutically acceptable salt of a compound of Series C-2 Formula I, and all of the remaining formulas disclosed herein is also contemplated.

In preferred embodiments, a compound of Series C-2 Formula I can be present as a pharmaceutically acceptable salt, and can optionally be present including both individual enantiomeric forms, a racemate, diastereomers and mixtures thereof.

In another preferred embodiment where R⁸ is H, one of n and m is zero and the remaining Z or Q is CH₂, a compound of Series C-2 Formula I has the structure of Series C-2 Formula II

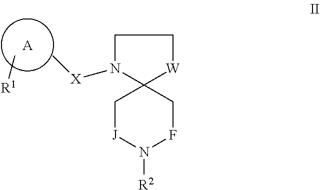

wherein J and F are the same or different and are CH₂, CHD or CD₂ (where D is deuterium); and

X, W, circle A, R¹, R² and the R groups therein defined are as described previously for a compound of Series C-2 Formula I, unless the formula as shown precludes a prior definition.

In a further preferred embodiment, where p is zero, a compound of Series C-2 Formula I has the structure of Series C-2 Formula III

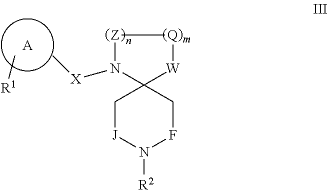

wherein J and F are the same or different and are CH₂, CHD or CD₂ (where D is deuterium);

each of m and n is one; and

W, X, Z, Q, circle A, R¹, R² and the R groups therein defined are as described previously for a compound of Series C-2 Formula I, unless the formula as shown precludes a prior definition.

In a still further preferred embodiment, i) Z is C(O), ii) Q is CH₂, iii) W is NH, (vi) R² is the same or different R²⁰, and (vii) R²⁰ is X-circle A-R¹. In this embodiment, X is preferably CH₂, SO₂, NHC(NH), NHC(S) or NHC(O), more preferably CH₂.

A presently most preferred compound for carrying out a contemplated method corresponds in structure to Formula III, above, in which i) Z is C(O), ii) Q is CH₂, iii) W is NH, iv) R² is H or a C₁-C₁₂, preferably C₁-C₈, and more preferably a C₁-C₆, aliphatic straight, branched or cyclic hydrocarbyl group, v) X is CH₂, and circle A-R¹ is unsubstituted phenyl so that the substituent X-circle A-R¹ is a benzyl group. Illustrative presently most preferred compounds include Compounds C0105M, C0115M and C0124M, whose structural formulas are shown below.

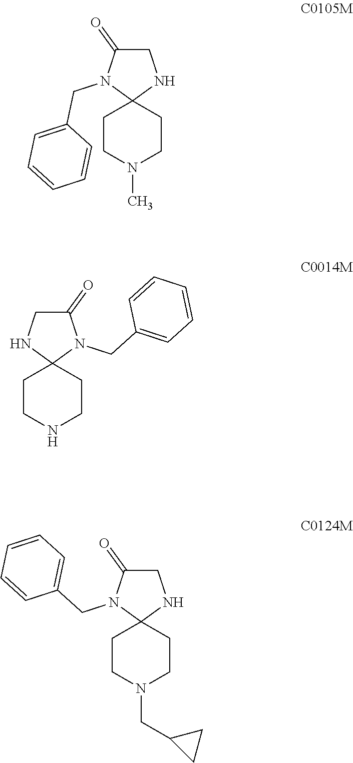 [SMILES: CN1CCC2(CC1)NCC(=O)N2Cc1ccccc1 ; O=C1CNC2(CCNCC2)N1Cc1ccccc1 ; O=C1CNC2(CCN(CC3CC3)CC2)N1Cc1ccccc1]

In preferred practice for the compounds of Series C-2 Formulas I, p=zero (0) so the central ring is a spiro 5,6-ring system whose 6-membered ring carbon atoms are unsubstituted except for the spiro-bonded carbon and possibly the nitrogen, and in which the spiro bonds are in the 4-position relative to the nitrogen of the 6-membered ring. It is separately preferred that W be O, or NR⁷. It is also preferred that X be SO₂ (sulfonyl) of CH₂ (methylene).

The aromatic substituent, the circle A, is linked to one nitrogen atom of the spiro rings by a X group that is SO₂, C(O), CH₂, CD₂, OC(═O), NHC(═NH), NHC(═S) or NHC(═O), preferably SO₂, C(O), CH₂, or CD₂, and most preferably CH₂ and SO_{2.} The resulting aromatic substituent is thereby linked to the spiro ring portion by a sulfonamide, an amide, a methylene, a urea, a thiourea or a guanidino linkage. Aryl sulfonamide bridges, aryl amide bridges and phenylmethylene bridges (benzyl compounds) are preferred, with aryl sulfonamide and phenylmethylene being particularly preferred.

A 1,4,8-triazaspiro[4,5]-decan-2-one compound of Series D corresponds in structure to the formula

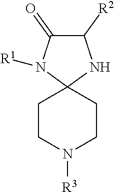

wherein R¹ represents hydrogen; a linear or branched unsubstituted or at least monosubstituted alkyl group that can comprise at least one heteroatom as a link; a linear or branched unsubstituted or at least monosubstituted alkenyl group that can comprise at least one heteroatom as a link; a linear or branched unsubstituted or at least monosubstituted alkynyl group that can comprise at least one heteroatom as a link; an unsubstituted or at least monosubstituted aryl group or an unsubstituted or at least monosubstituted heteroaryl group, which aryl and heteroaryl groups may be bonded via a linear or branched alkylene group that can comprise at least one heteroatom as a link; or a —C(═O)OR⁷ group that can be bonded via a linear or branched alkylene group;

R² represents hydrogen; a linear or branched unsubstituted or at least monosubstituted alkyl group that can comprise at least one heteroatom as a link; a linear or branched unsubstituted or at least monosubstituted alkenyl group that can comprise at least one heteroatom as a link; a linear or branched unsubstituted or at least monosubstituted alkynyl group that can comprise at least one heteroatom as a link; an unsubstituted or at least monosubstituted aryl group or an unsubstituted or at least monosubstituted heteroaryl group, which aryl and heteroaryl group may be bonded via a linear or branched alkylene group that can comprise at least one heteroatom as a link;

R³ represents a —S(═O)₂—R⁴ group; a —C(═S)NH—R⁵ group; or a —C(═O)NH—R⁶ group;

R⁴ represents a —NR¹⁰R¹¹ group; a linear or branched unsubstituted or at least monosubstituted alkyl group that can comprise at least one heteroatom as a link; a linear or branched unsubstituted or at least monosubstituted alkenyl group that can comprise at least one heteroatom as a link; a linear or branched unsubstituted or at least monosubstituted alkynyl group that can comprise at least one heteroatom as a link; an unsubstituted or at least monosubstituted aryl group or an unsubstituted or at least monosubstituted heteroaryl group, which groups may be bonded via a linear or branched unsubstituted or at least monosubstituted alkylene group that can comprise at least one heteroatom as a link and may be condensed with an unsubstituted or at least monosubstituted monocyclic ring system; an unsubstituted or at least monosubstituted cycloaliphatic group, that can comprise at least one heteroatom as a ring member and that can be bonded via a linear or branched unsubstituted or at least monosubstituted alkylene group that can comprise at least one heteroatom as a link and that can be bridged by a linear or branched unsubstituted or at least monosubstituted alkylene group;

R⁵ represents a linear or branched unsubstituted or at least monosubstituted alkyl group that can comprise at least one heteroatom as a link; a linear or branched unsubstituted or at least monosubstituted alkenyl group that can comprise at least one heteroatom as a link; a linear or branched unsubstituted or at least monosubstituted alkynyl group that can comprise at least one heteroatom as a link; an unsubstituted or at least monosubstituted aryl group or an unsubstituted or at least monosubstituted heteroaryl group, which group may be bonded via a linear or branched unsubstituted or at least monosubstituted alkylene group that can comprise at least one heteroatom as a link; an unsubstituted or at least monosubstituted cycloaliphatic group, that can comprise at least one heteroatom as a ring member or that can be bonded via a linear or branched unsubstituted or at least monosubstituted alkylene group that can comprise at least one heteroatom as a link; a —C(═O)OR⁸ group or a —C(═O)OR⁹ group, that can, in either case, be bonded via a linear or branched alkylene group;

R⁶ represents an unsubstituted or at least monosubstituted aryl group or an unsubstituted or at least monosubstituted heteroaryl group, which aryl and heteroaryl groups may be bonded via a linear or branched unsubstituted or at least monosubstituted alkylene group that can comprise at least one heteroatom as a link; or for an unsubstituted or at least monosubstituted cycloaliphatic group, that can comprise at least one heteroatom as a ring member or that can be bonded via a linear or branched unsubstituted or at least monosubstituted alkylene group that can comprise at least one heteroatom as a link;

R⁷, R⁸, R⁹, R¹⁰, and R¹¹, each independently represent a linear or branched alkyl group, a linear or branched alkenyl group, or a linear or branched alkynyl group, or a physiologically acceptable salt thereof.

Preferably for a 1,4,8-triazaspiro[4,5]-decan-2-one compound corresponding to the formula above , R¹ represents hydrogen; a linear or branched unsubstituted or at least monosubstituted C₁₋₁₀ alkyl group that can comprise at least one heteroatom as a link; a linear or branched unsubstituted or at least monosubstituted C₂₋₁₀ alkenyl group that can comprise at least one heteroatom as a link; a linear or branched unsubstituted or at least monosubstituted C₂₋₁₀ alkynyl group that can comprise at least one heteroatom as a link; an unsubstituted or at least monosubstituted five-membered to fourteen-membered aryl group or heteroaryl group, that can be bonded via a linear or branched C₁₋₅ alkylene group that can comprise at least one heteroatom as a link; a —C═O)OR⁷ group that can be bonded via a linear or branched C₁₋₅ alkylene group;

R² represents hydrogen; a linear or branched unsubstituted or at least monosubstituted C₁₋₁₀ alkyl group that can comprise at least one heteroatom as a link; a linear or branched unsubstituted or at least monosubstituted C₂₋₁₀ alkenyl group that can comprise at least one heteroatom as a link; a linear or branched unsubstituted or at least monosubstituted C₂₋₁₀ alkynyl group that can comprise at least one heteroatom as a link; an unsubstituted or at least monosubstituted five-membered to fourteen-membered aryl or heteroaryl group, that can be bonded via a linear or branched C₁₋₅ alkylene group that can comprise at least one heteroatom as a link;

R⁴ represents an NR¹⁰R¹¹ group; a linear or branched unsubstituted or at least monosubstituted C₁₋₁₀ alkyl group that can comprise at least one heteroatom as a link; a linear or branched unsubstituted or at least monosubstituted C₂₋₁₀ alkenyl group that can comprise at least one heteroatom as a link; a linear or branched unsubstituted or at least monosubstituted C₂₋₁₀ alkynyl group that can comprise at least one heteroatom as a link; an unsubstituted or at least monosubstituted five-membered to fourteen-membered aryl group or heteroaryl group, that can be bonded via a linear or branched unsubstituted or at least monosubstituted C₁₋₅ alkylene group that can comprise at least one heteroatom as a link and may be condensed with a five-membered or six-membered monocyclic ring system; an unsubstituted or at least monosubstituted C₃₋₈-cycloaliphatic group that can comprise at least one heteroatom as a ring member or that can be bonded via a linear or branched unsubstituted or at least monosubstituted C₁₋₅ alkylene group that can comprise at least one heteroatom as a link and that can be bridged by a linear or branched unsubstituted or at least monosubstituted C.sub.1-5 alkylene group;

R⁵ represents a linear or branched unsubstituted or at least monosubstituted C₁₋₁₀ alkyl group that can comprise at least one heteroatom as a link; a linear or branched unsubstituted or at least monosubstituted C₂₋₁₀ alkenyl group that can comprise at least one heteroatom as a link; a linear or branched unsubstituted or at least monosubstituted C₂₋₁₀ alkynyl group that can comprise at least one heteroatom as a link; an unsubstituted or at least monosubstituted five-membered to fourteen-membered aryl or heteroaryl group, that can be bonded via a linear or branched unsubstituted or at least monosubstituted C₁₋₅ alkylene group that can comprise at least one heteroatom as a link; an unsubstituted or at least monosubstituted C₃₋₈-cycloaliphatic group that can comprise at least one heteroatom as a ring member and that can be bonded via a linear or branched unsubstituted or at least monosubstituted C₁₋₅ alkylene group that can comprise at least one heteroatom as a link; a —C(═O)OR⁸ group or a —C(═O)OR⁹ group either of that can be bonded via a linear or branched C₁₋₁₀ alkylene group;

R⁶ represents an unsubstituted or at least monosubstituted five-membered to fourteen-membered aryl or heteroaryl group, which aryl or heteroaryl group may be bonded via a linear or branched unsubstituted or at least monosubstituted C₁₋₅ alkylene group that can comprise at least one heteroatom as a link; an unsubstituted or at least monosubstituted C₃₋₈-cycloaliphatic group that can comprise at least one heteroatom as a ring member, or that can be bonded via a linear or branched unsubstituted or at least monosubstituted C₁₋₅ alkylene group that can comprise at least one heteroatom as a link; and

R⁷, R⁸, R⁹, R¹⁰, and R¹¹, independently represent a linear or branched C₁₋₅ alkyl group, a linear or branched C₂₋₅ alkenyl group, or a linear or branched C₂₋₅ alkynyl group.

Compounds A, B and C whose structural formulas are shown below are illustrative preferred compounds of Series D.

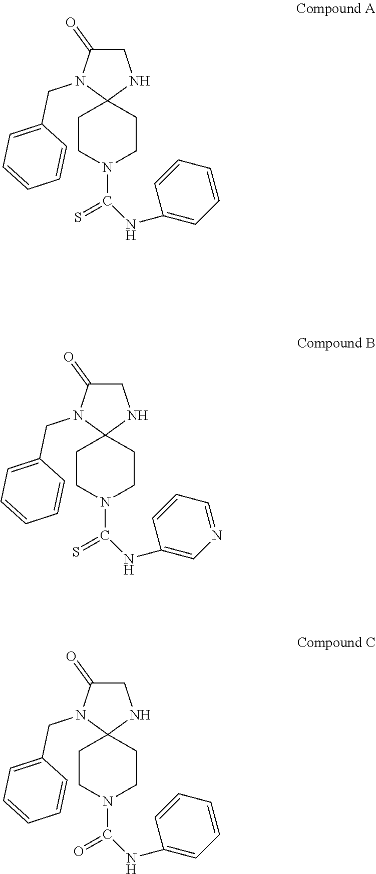 [SMILES: O=C1CNC2(CCN(C(=S)Nc3ccccc3)CC2)N1Cc1ccccc1 ; O=C1CNC2(CCN(C(=S)Nc3cccnc3)CC2)N1Cc1ccccc1 ; O=C(Nc1ccccc1)N1CCC2(CC1)NCC(=O)N2Cc1ccccc1]

Many of the compounds of Series C-1, Series C-2, and Series D as well as compounds such as naloxone and naltrexone not only bind to the FLNA pentapeptide of positions 2561-2565, but also bind to MOR and activate or stimulate that receptor. Naloxone and naltrexone bind to MOR about 200 times more poorly than they bind to the pentapeptide of the above FLNA pentapeptide. The tables of Example 2 illustrate relative binding abilities of exemplary compounds of Series C-1, and Series C-2 based on MOR stimulatory activity.

In some embodiments it is preferred that a compound useful in a contemplated method binds well to and activates MOR. In those cases, it is preferred that the compound bind to MOR to an extent of at least about ±20 percent as well as DAMGO at a concentration shown in the tables, indicating the compound is a complete agonist for the receptor. In other embodiments, it is preferred that a compound useful herein not bind well to MOR. In those embodiments, it is preferred that the compound exhibit less than about 80 percent the MOR stimulation provided by DAMGO at the same concentration and conditions, down to zero binding/stimulation. Illustrative binding percentages in the presence of stated concentrations of DAMGO are illustrated for exemplary compounds of Series C-1 and Series C-2 in the tables of Example 2, hereinafter.

### Pharmaceutical Compositions

A contemplated compound useful in the invention can be provided for use by itself, or as a pharmaceutically acceptable salt. Regardless of whether in the form of a salt or not, a contemplated compound is typically dissolved or dispersed in a pharmaceutically acceptable diluent that forms a pharmaceutical composition and that pharmaceutical composition is administered to mammalian cells in recognized (diagnosed) need.

A contemplated compound or its pharmaceutically acceptable salt can be used in the manufacture of a medicament (pharmaceutical composition) that is useful at least for inhibiting one or more of a mammalian cell surface receptor-mediated immune response in cells in recognized (diagnosed) need thereof and expressing one or more of TLR2, RAGE, CCR5, CXR4 and CD4 cell surface receptors. Cells in recognized need are those cells that express at least one standard deviation more than the normally present amount of one or more of such receptors in cells that are not inflamed, or express one or more inflammatory cytokines or chemokines as previously discussed that are present in an amount that is at least one standard deviation greater than the amount normally present the same types of cells that are not inflamed, as was previously discussed.

A contemplated pharmaceutical composition contains an effective amount of a contemplated compound or a pharmaceutically acceptable salt thereof dissolved or dispersed in a physiologically tolerable carrier. Such a composition can be administered to mammalian cells in vitro as in a cell culture, or in vivo as in a living, host mammal in need.

A contemplated composition is typically administered a plurality of times over a period of days. More usually, a contemplated composition is administered once or twice daily. It is contemplated that once administration of a contemplated compound has begun the compound will be administered chronically for the duration of the study being carried out or for a recipient's lifetime.

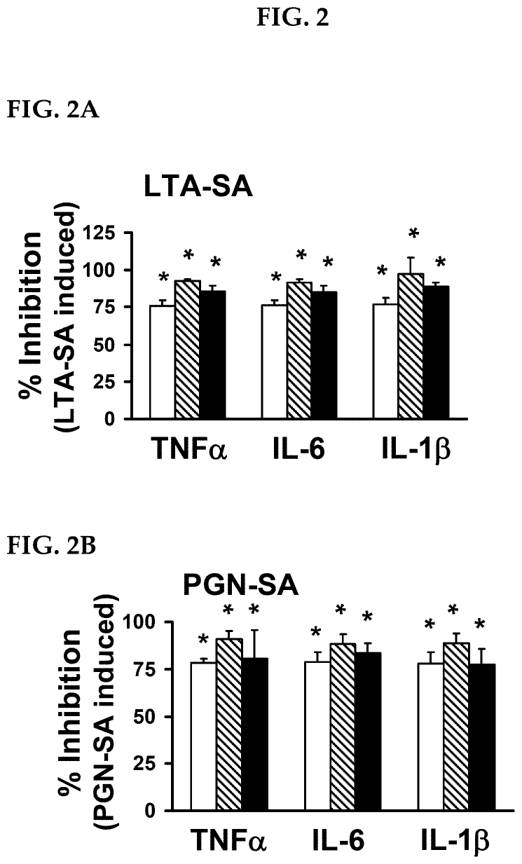

A contemplated compound can bind to FLNA at a 100 femtomolar concentration and effectively inhibits cytokine release from TLR2-stimulated astrocytes in vitro (see, FIGS. 2A and 2B). A contemplated compound is more usually utilized at picomolar to micromolar amounts.

Thus, an effective amount of a contemplated compound present in a contemplated pharmaceutical composition is that which provides a concentration of about 100 femtomolar to about 1 micromolar to a host animal's blood stream or to an in vitro cell medium in practicing a contemplated method of the invention. A more usual amount is about 1 picomolar to about 1 micromolar. A still more usual amount is about 1 picomolar to about 1 nanomolar.

Looked at differently, tableted dosages of about 25 to about 200 mg twice a day for an adult human and more preferably about 50 to about 100 mg twice a day has been found to reduce the inflammatory effects of Alzheimer's disease in adult human patients in two clinical studies. The prior dosages can also be spread out to be given more frequently in smaller amounts as ordered by a treating physician.

The efficacy of the contemplated compounds at low nM concentrations indicates a large window for therapeutic efficacy for use of a contemplated compound. Thus, a skilled worker can readily determine an appropriate dosage level of a contemplated compound to inhibit a desired amount of inflammatory response.

A contemplated pharmaceutical composition can be administered orally (perorally), parenterally, by inhalation spray in a formulation containing conventional nontoxic pharmaceutically acceptable carriers, adjuvants, and vehicles as desired. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

For injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution, phosphate-buffered saline. Liquid pharmaceutical compositions include, for example, solutions suitable for parenteral administration. Sterile water solutions of an active component or sterile solution of the active component in solvents comprising water, ethanol, or propylene glycol are examples of liquid compositions suitable for parenteral administration.

In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Solid dosage forms for oral administration can include capsules, tablets, pills, powders, and granules. In such solid dosage forms, a contemplated compound is ordinarily combined with one or more excipients appropriate to the indicated route of administration.

If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets, capsules and pills can additionally be prepared with enteric coatings.

A mammal in need of treatment and to which a pharmaceutical composition containing a contemplated compound is administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like. Where in vitro mammalian cell contact is contemplated, a tissue culture of cells from an illustrative mammal is often utilized, as is illustrated hereinafter.

Preferably, the pharmaceutical composition is in unit dosage form. In such form, the composition is divided into unit doses containing appropriate quantities of the active agent. The unit dosage form can be a packaged preparation, the package containing discrete quantities of the preparation, for example, in vials or ampules.

Several useful contemplated compounds are amines and can typically be used in the form of a pharmaceutically acceptable acid addition salt derived from an inorganic or organic acid. Exemplary salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenylpropionate, picrate, pivalate, propionate, succinate, tartrate, thiocyanate, tosylate, mesylate and undecanoate.

Other compounds useful in this invention that contain acid functionalities can also form salts with a base. Illustrative bases include amine bases such as mono-, di- and tri-C₁-C₄-alkyl or hydroxyalkyl amines like triethyl amine, dimethylamine, 2-hydroxyethylamine, and dimethyl-2-hydroxyethylamine, and bases such as alkali metal, alkaline earth metal quaternary C₁-C₆-alkyl ammonium hydroxides, such as sodium, potassium, calcium, magnesium and tetramethylammonium hydroxides. Basic salts such as alkali metal or alkaline earth metal and ammonium carbonates and phosphates are also contemplated.

The reader is directed to Berge, J. Pharm. Sci. 68(1):1-19 (1977) for lists of commonly used pharmaceutically acceptable acids and bases that form pharmaceutically acceptable salts with pharmaceutical compounds.

In some cases, the salts can also be used as an aid in the isolation, purification or resolution of the compounds of this invention. In such uses, the acid used and the salt prepared need not be pharmaceutically acceptable.

Discussion

The discussion that follows illustrates compounds and compositions that contain one or more of those compounds that bind the scaffolding protein FLNA, and particularly the FLNA pentapeptide binding site present in the FLNA protein of positions 2561-2565. A Compound of such a composition also disrupts the toxic signaling of amyloid-b₄₂ (Ab₄₂). These compounds diminish many aspects of AD-like pathology, including impairments in normal receptor functioning, as well as diminishing an inflammatory response caused by viral or bacterial infection such as sepsis or the so-called cytokine storm that can result from such infections.

Initial studies were carried out with compounds of two structural series using Compounds C0105, C0114, C0137 and C0138 as illustrative or exemplary. Additional studies were also carried out using Compounds C0134, Compound A, Compound B, and Compound C, the latter three being members of Series D. The results shown in the Figures and discussed hereinafter are indicative of the generality of results obtained using these structurally very different compounds. Initial results indicated that the compounds appear to be orally available and well tolerated because notable plasma and CNS levels were produced but negligible side effects were noted at 2 g/kg administered orally in rats. Those results also indicated similar activities among the nine compounds, with Compounds C0105 and C0114 being used for further studies because of their high activity, ease of synthesis, solubility and absence of enantiomers.

The fact that Ab₄₂ binding blocks Ca⁺² influx by a7nAChRs [Wang et al., J Neurosci 35:10961-10973 (2009); Wang et al., Biol Psychiatry 67:522-530 (2010)] suggests that one conformational change in a7nAChRs may occur in the interface between extracellular and transmembrane domains, the area governing channel opening/desensitization [Bouzat et al., J Neurosci 28:7808-7819 (2008)]. This conformational change likely exposes a positive charge-rich transmembrane region close to the Ab₄₂ binding site. FLNA binds this positive charge to stabilize the bound Ab₄₂ and additional binding of Ab₄₂ peptides, leading to eventual internalization of Ab₄₂-a7nAChR complexes [(Nagele et al., Neuroscience 110:199-211 (2002)]. Compound C0105 disruption of the FLNA-a7nAChR interaction stops the pathological signaling and stops Ab₄₂ high-affinity anchoring to the receptor.

Using organotypic frontocortical slice cultures of adult rats, Ab signaling through the a7 nicotinic acetylcholine receptor (a7nAChR) is shown to require the recruitment of FLNA. By binding a critical pentapeptide segment of FLNA, these compounds block the FLNA-a7nAChR association and the signaling cascade of Ab₄₂. In the illustrative Ab₄₂-treated organotypic frontocortical slice cultures, exemplary Compound C0105 dramatically reduces phosphorylation of tau at all three phosphorylation sites of tau found in neurofibrillary tangles (FIGS. 12A, 12B and 12C).

Lipoteichoic acid from S. aureus (LTA-SA) and peptidoglycan from S. aureus (PGN-SA) are activating ligands for TLR2. FIGS. 10A and 10B illustrate that each causes insult-induced release of pro-inflammatory cytokines IL-1b, IL-6 and TNFa from human astrocytes. Treating those human astrocytes with an effective amount of illustrative Compound 105 along with the insulting ligand substantially inhibited the release of each of those three pro-inflammatory ligands.

The anti-inflammatory effect of illustrative Compound C0105 is believed to occur by a disruption of Ab₄₂-induced FLNA association with TLR2. Ab₄₂ increases FLNA association with TLR2, and this association appears to be critical to inflammatory cytokine production due to Ab₄₂ exposure, because illustrative Compound C0105 nearly abolishes this cytokine production. Although Ab₄₂ does not itself interact with TLR2, Ab₄₂ binds to CD14, which in turn binds TLR2 to produce the inflammation noted in AD [Reed-Geaghan et al., J. Neurosci. 29(38):11982-11992 (Sep. 23, 2009)].

It is believed that illustrative Compound C0105 prevents an Ab₄₂-induced association of FLNA with TLR2. Disruption of that association is the probable mechanism of action for anti-inflammatory effects of our FLNA-binding compounds [Burns et al., Recent Patents on CNS Drug Discovery 5:210-220 (2010)].

A recent paper by the inventors and their co-workers [Wang et al., Neurobiol Aging 55:99-114 (2017)] showed that pI of FLNA from the brains of naïve mice compared to that from Ab₄₂-infused, 3×Tg AD and aged wild-type mice shifted from 5.9 to 5.3, and could be returned to 5.9 by in vivo treatment of Ab₄₂-infused, 3×Tg AD and aged wild-type mice with Illustrative Compound 105 at 20-22 mg/kg. These results were interpreted as evidencing an altering of the FLNA conformation from that induced by Ab₄₂-infusion, 3×Tg AD and age to that of the younger, naïve mice. It is believed that the inhibition of TLR2-, CCR5-, CXCR4-, and CD4-mediated inflammation described herein is a result of a similar change in conformation induced by binding with a contemplated compound that disrupts the interaction of FLNA and its binding partner that leads to the perceived inflammation.

### Specific Results

Effects on Release of Pro-Inflammatory Cytokines (IL-1b, IL-6 and TNFa) Induced from Primary Human Astrocytes by Contact with Ab₄₂, LPS, LTA (Lipoteichoic Acid)-SA and PGN (Peptidoglycan)-SA

Human astrocytes express both the TLR4 and TLR2 cell surface receptors. Ab₄₂ and LPS each bind to and activate the TLR4 signaling pathway resulting in the release of pro-inflammatory cytokines such as IL-1b, IL-6 and TNFa, as is shown in previous studies discussed herein. It was of interest to determine whether LTA-SA (S. aureus LTA) and/or PGN-SA (S. aureus peptidoglycan) that bind to TLR2 would also activate TLR2 signaling, inducing the release of the same and/or different pro-inflammatory cytokines. It was also of interest to assay whether illustrative Compound C0105 that inhibits that cytokine signaling by TLR4 would act similarly toward TLR2, presuming that binding to that receptor by the above ligands also induces pro-inflammatory cytokine release.

### Experimental Design:

A primary astrocyte culture was prepared according to the provider (Lonza). The adherent astrocytes were trypsinized by 0.25% trypsin-EDTA, then collected and sub-cultured in 12-well plate (1.2 ml/well). When the cells were 80-85% confluent, cells were treated in an incubator under 5% CO₂ with 100 fM, 10 pM or 1 nM Compound C0105 immediately followed by the addition of Ab₄₂ (0.1 mM), LPS (1 mg/ml), LTA-SA (1 mg/ml) and PGN-SA (10 mg/ml); i.e., simultaneously adding the insulting ligand and Compound C0105 to the cells. Vehicle groups were treated with 0.1% DMSO only. Incubation continued for 24 hours post addition. Culture medium was used as the blank (non-treat) and the levels of cytokines, TNF-a, IL-6 and IL-1b in 200 ml of culture medium were determined. Each well was sampled once.

To determine the effect of Compound C0105 on cytokine release from human astrocytes, 0.5 mg/well biotinylated mouse monoclonal anti-TNF-a, anti-IL-6 and anti-IL-1b were coated onto streptavidin-coated plates (Reacti-Bind™ NeutrAvidin™ High binding capacity coated 96-well plates). Plates were washed 3 times with ice-cold 50 mM Tris HCl (pH 7.4) and incubated at 30° C. with 200 ml medium derived from the above-mentioned conditions. Plates were washed 3 times with ice-cold 50 mM Tris HCl (pH 7.4) and incubated at 30° C. with 0.5 mg/well un-conjugated rabbit anti-TNF-a, -IL-6 and -IL-1b for 1 hour. After three 1 minute washes with 50 mM Tris HCl (pH 7.4), each well was incubated in 0.25 mg/well FITC-conjugated anti-rabbit IgG (human and mouse absorbed) for 1 hour at 30° C. Plates were washed twice with 200 ml ice-cold Tris HCl, pH 7.4 and the residual FITC signals were determined by multimode plate reader, DTX880 (Beckman).

The results of these studies are shown in FIG. 2A for LTA-SA and FIG. 2B for PGN-SA. As can be seen, Compound C0105 inhibited release of each of the assayed cytokines by about 75 to about 95 percent for each of the three cytokines and each of the four ligands. Statistical analysis by one-way ANOVA: p<0.01; p*<0.01 compared to vehicle treated group for each insult.

Effects of LTA-SA- and PGN-SA-Ab₄₂-Induced Expression of TLR2 and FLNA, Inhibition of that Expression by Compound C0105 and Tau Phosphorylation and Inhibition from Human Postmortem Human Frontal Cortical Slices

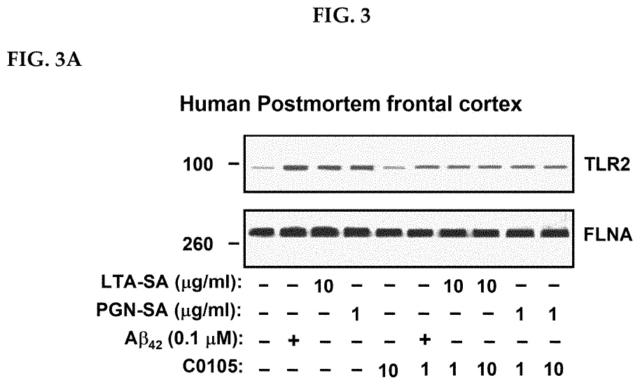

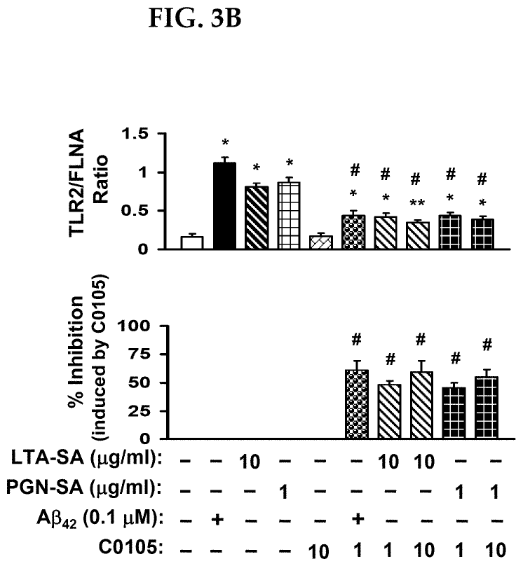

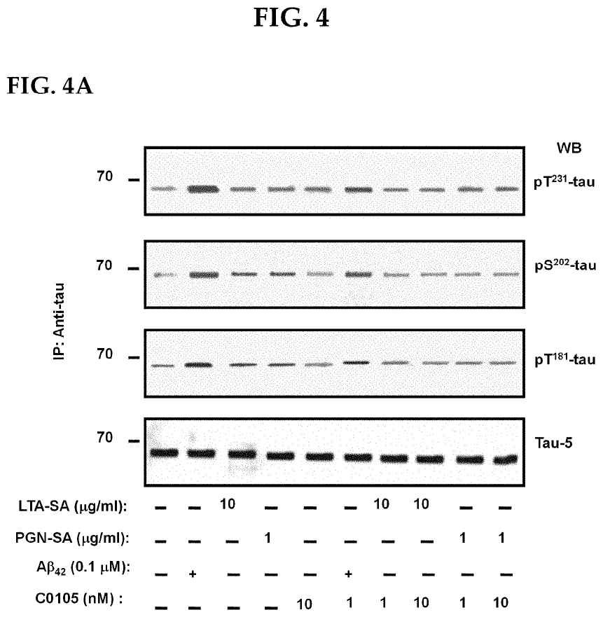

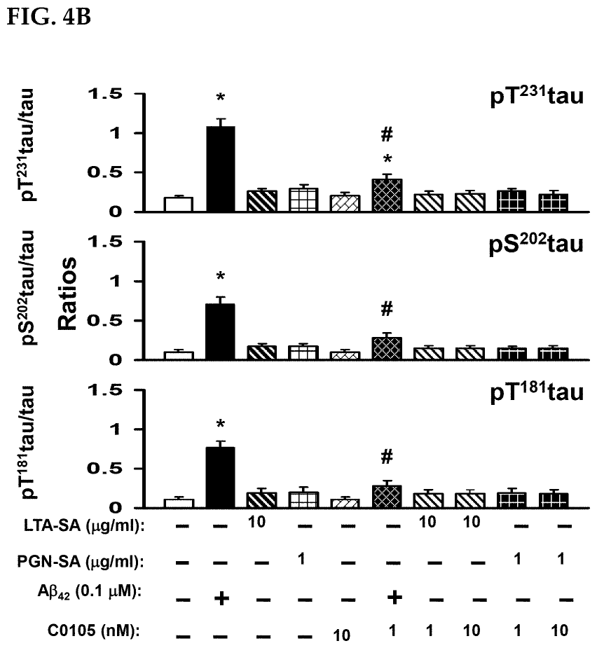

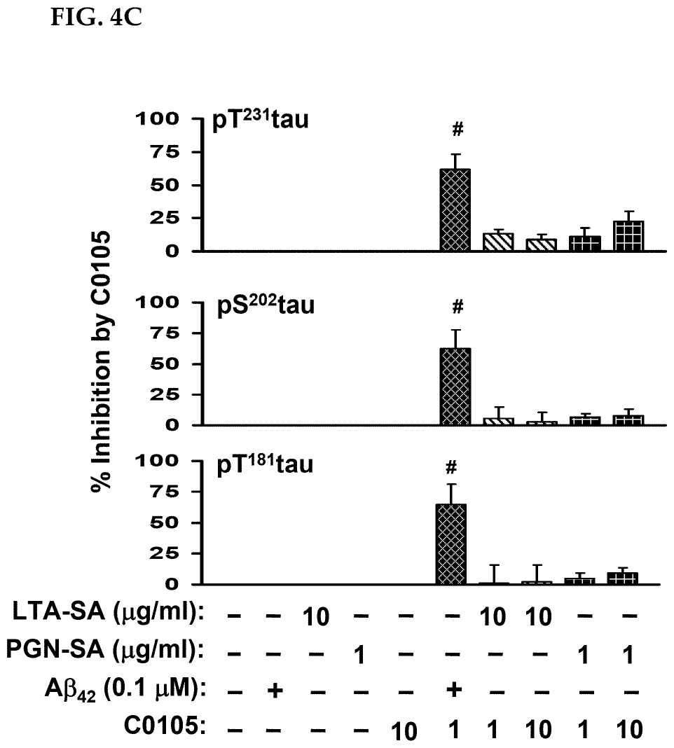

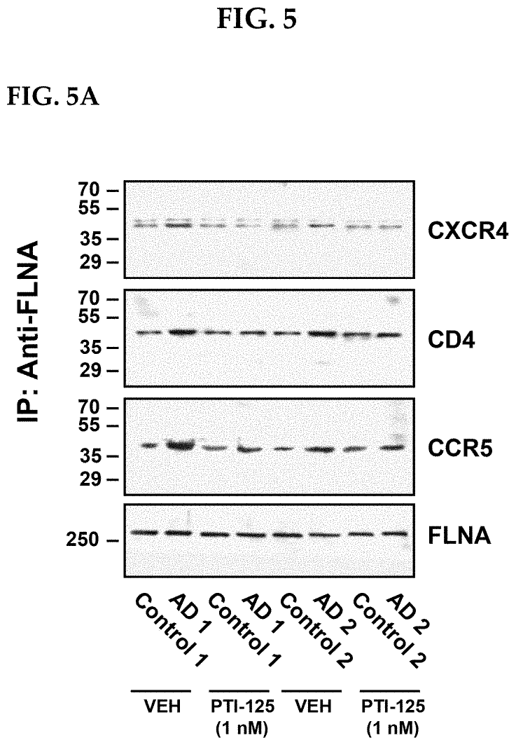

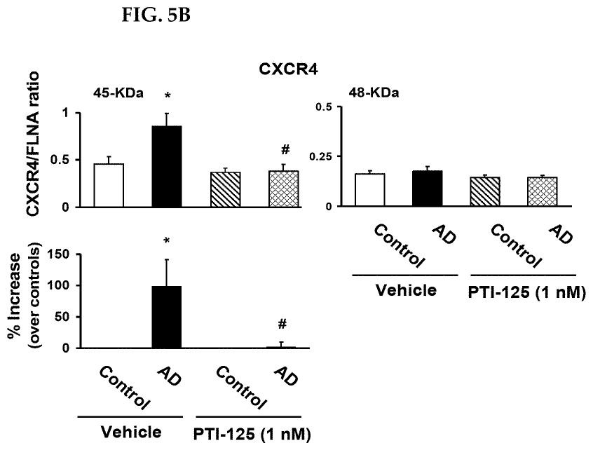

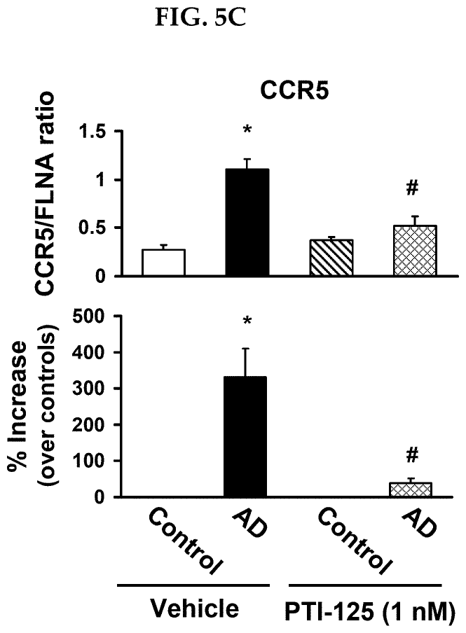

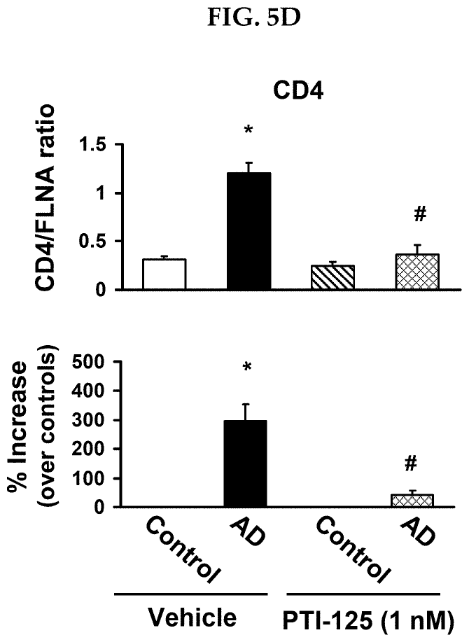

Human postmortem frontal cortex slices prepared as described above were treated with 10 mg/ml LTA-SA, 1 mg/ml PGN-SA or 0.1 mM Ab₄₂ to examine possible changes in expression of TLR2 and FLNA, and to examine possible changes in the phosphorylation of tau, as well as the inhibition of both processes by Compound C0105. As will be seen from examination of FIG. 3 and FIG. 4, the ratio of TLR2 to FLNA increased with administration of each of LTA-SA, PGN-SA and Ab₄₂ at a statistically significant amount relative to the control (p<0.01). Those changes in expression of TLR2 relative to FLNA were each inhibited by the presence of the 1 or 10 nM Compound C0105 at a statistically significant amount relative to the control (p<0.01) and relative to the ligand used. See, FIG. 3B.

Tau phosphorylation appeared to be slightly elevated due to the presence of LTA-SA or PGN-SA, and significantly by Ab₄₂. A slight inhibition (about 25% or less) of that enhanced tau phosphorylation was provided by contacting the cells with 1 or 10 nM Compound C0105. A statistically significant reduction in Ab₄₂-induced tau phosphorylation was also observed. See, FIGS. 4A and 4B.

### Clinical Studies

Series C-2 Compound C0105 (Pti-125; sumifilam) has been undergoing a series of clinical studies in the treatment of Alzheimer's disease and has shown a reduction in the immunoinflammatory cytokines in treated patients. These patients were administered 50 or 100 mg of the compound twice daily in an open label 28-day study and also in an open label study whose results over 6 months of treatment.

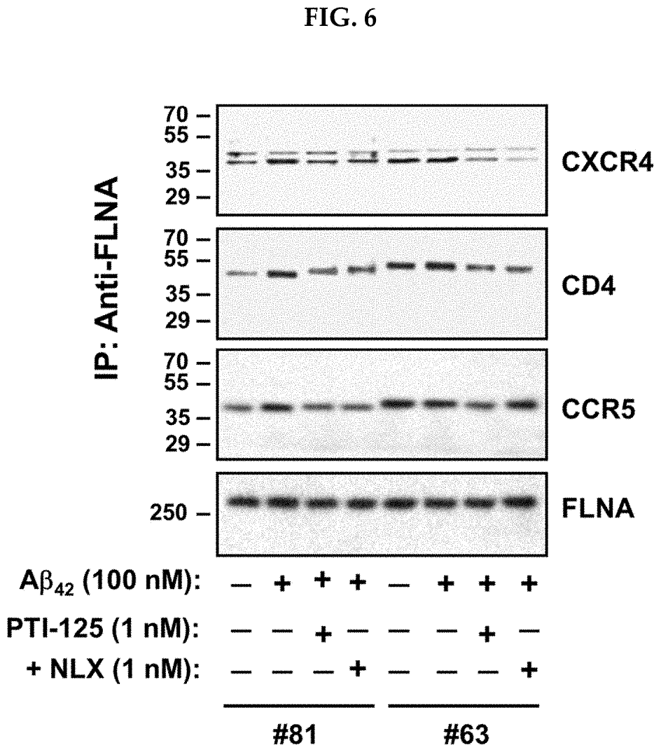

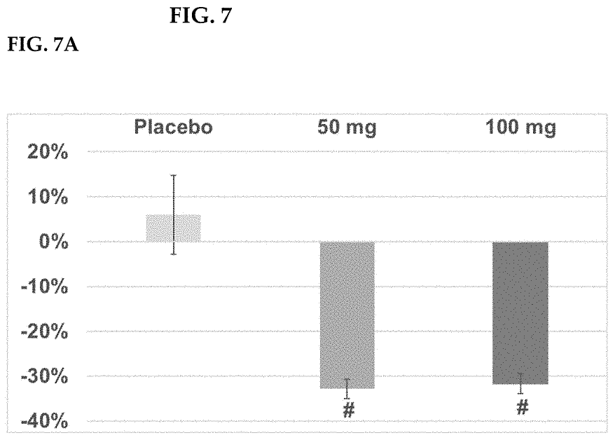

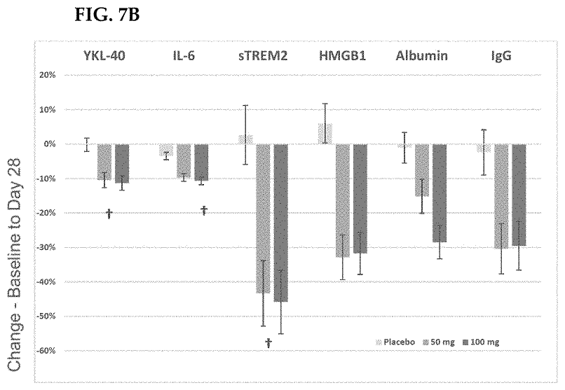

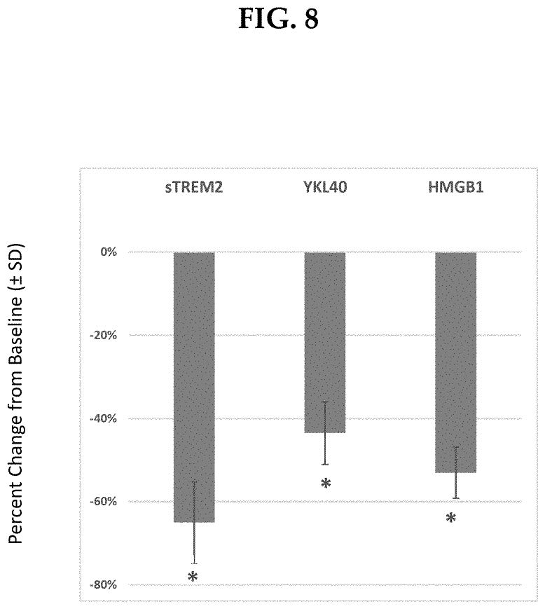

Results related to the lessening of inflammation mediated by one or more of TLR2, RAGE, CCR5, CXR4 and CD4 cell surface receptors are shown in FIGS. 6, 7 and 8 and their sub-parts. As is seen, this treatment provided a statistically significant anti-inflammatory effect on the treated patients.

### Compounds

Compounds were synthesized and provided by Medicilon, Shanghai. Aside from the three syntheses described herein, more detailed syntheses are set out in one or more of U.S. Pat. Nos. 8,722,851 B2, 8,580,808 B2 (or one or more of U.S. Patent publications 2010/0279996 A1, No. 2010/0279997 A1, No. 2010/0280061 A1, No. 2011/0105481 A1, 2011/0105484 A1), U.S. Pat. Nos. 8,653,068 B2, 8,580,809 B2, and 10,017,736, whose disclosures are incorporated by reference.

A compound having an asymmetrical (chiral) carbon or a salt thereof can exist in the form of two enantiomers. The invention relates both to each enantiomer and to their mixture; i.e., to both enantiomeric forms and to their mixture. Additionally, where two or more chiral centers are present, diastereomers can form.

Where a contemplated compound or a pharmaceutically acceptable salt of a compound of Series C-1, C-2, or D, or any of the other formulas herein is obtained in the form of a mixture of the stereoisomers, preferably in the form of the racemates or other mixtures of the various enantiomers and/or diastereoisomers, they can be separated and optionally isolated by conventional methods known to the person skilled in the art. Illustratively, chromatographic separation processes are useful, particularly liquid chromatography processes under standard pressure or under elevated pressure, preferably MPLC and HPLC methods, and also methods involving fractional crystallization. This can particularly involve the separation of individual enantiomers, e.g., diastereoisomeric salts separated by means of HPLC in the chiral phase or by means of crystallization with chiral acids, for example (+)-tartaric acid, (−)-tartaric acid, or (+)-10-camphorsulfonic acid. An enantiomer separated by chiral salt formation can readily be converted into an achiral or racemic pharmaceutically acceptable salt for use.

A compound of Series C-1, C-2, or D or a pharmaceutically acceptable salt thereof is contemplated to be optionally used in a process of the invention in enantiomerically pure form; i.e., in (S) or (R) configuration or d and l forms, or in the form of a racemic mixture showing an (S,R) or (d,l) configuration, or as one or more diastereomers, and mixtures thereof.

Thus, a contemplated compound or its pharmaceutically acceptable salt can optionally be present in one or more forms. Illustratively, the compound or its salt can be in the form of an individual enantiomer or diastereoisomer. A contemplated compound or its salt can also be present in the form of a mixture of stereoisomers. A contemplated compound or salt can also be present in the form of a racemic mixture.

Table of Series-C-1 Compounds

| | |
|---|---|
| 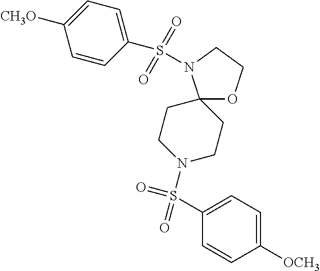 | 7866 |
| 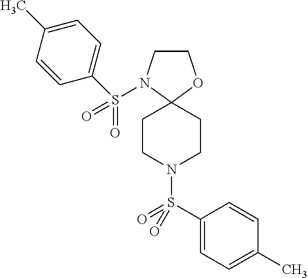 | C0001 |
| 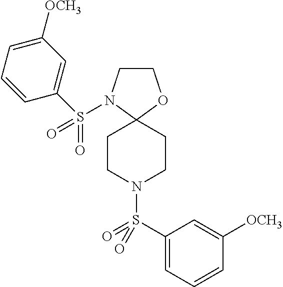 [SMILES: COc1cccc(S(=O)(=O)N2CCC3(CC2)OCCN3S(=O)(=O)c2cccc(OC)c2)c1] | C0002 |
| 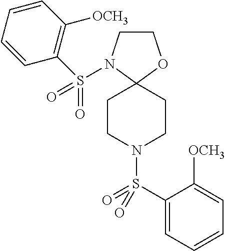 [SMILES: COc1ccccc1S(=O)(=O)N1CCC2(CC1)OCCN2S(=O)(=O)c1ccccc1OC] | C0003 |
| 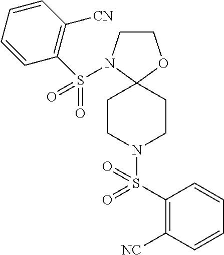 [SMILES: N#Cc1ccccc1S(=O)(=O)N1CCC2(CC1)OCCN2S(=O)(=O)c1ccccc1C#N] | C0004 |
| 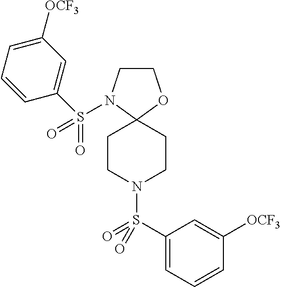 [SMILES: O=S(=O)(c1cccc(OC(F)(F)F)c1)N1CCC2(CC1)OCCN2S(=O)(=O)c1cccc(OC(F)(F)F)c1] | C0005 |
| 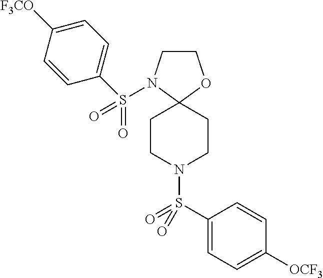 [SMILES: O=S(=O)(c1ccc(OC(F)(F)F)cc1)N1CCC2(CC1)OCCN2S(=O)(=O)c1ccc(OC(F)(F)F)cc1] | C0006 |
| 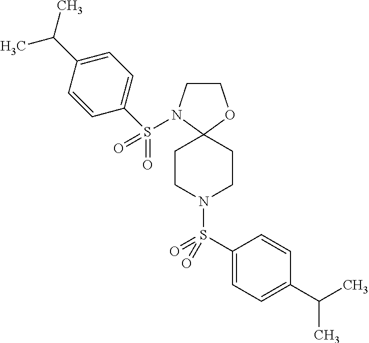 [SMILES: CC(C)c1ccc(S(=O)(=O)N2CCC3(CC2)OCCN3S(=O)(=O)c2ccc(C(C)C)cc2)cc1] | C0007 |
| 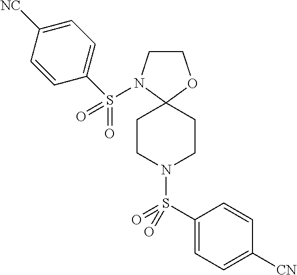 [SMILES: N#Cc1ccc(S(=O)(=O)N2CCC3(CC2)OCCN3S(=O)(=O)c2ccc(C#N)cc2)cc1] | C0008 |
| 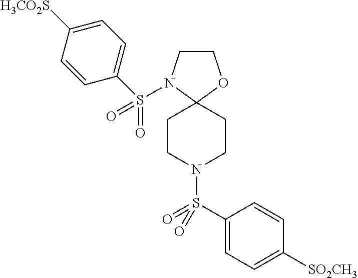 | C0009 |
| 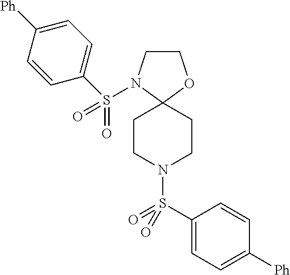 [SMILES: O=S(=O)(c1ccc(-c2ccccc2)cc1)N1CCC2(CC1)OCCN2S(=O)(=O)c1ccc(-c2ccccc2)cc1] | C0010 |
| 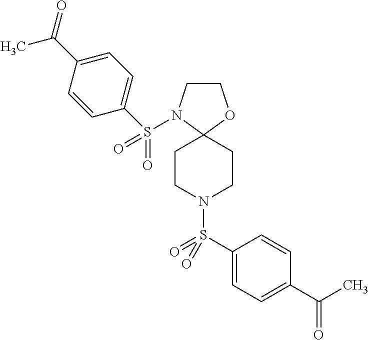 [SMILES: CC(=O)c1ccc(S(=O)(=O)N2CCC3(CC2)OCCN3S(=O)(=O)c2ccc(C(C)=O)cc2)cc1] | C0011 |
| 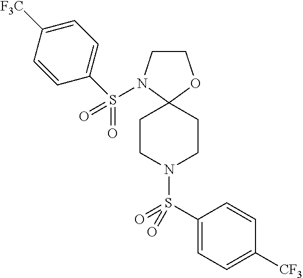 [SMILES: O=S(=O)(c1ccc(C(F)(F)F)cc1)N1CCC2(CC1)OCCN2S(=O)(=O)c1ccc(C(F)(F)F)cc1] | C0012 |
| 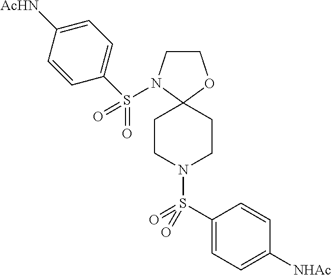 [SMILES: CC(=O)Nc1ccc(S(=O)(=O)N2CCC3(CC2)OCCN3S(=O)(=O)c2ccc(NC(C)=O)cc2)cc1] | C0013 |
| 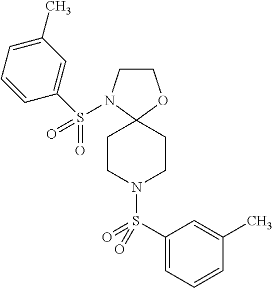 [SMILES: Cc1cccc(S(=O)(=O)N2CCC3(CC2)OCCN3S(=O)(=O)c2cccc(C)c2)c1] | C0014 |
| 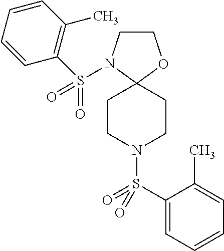 [SMILES: Cc1ccccc1S(=O)(=O)N1CCC2(CC1)OCCN2S(=O)(=O)c1ccccc1C] | C0015 |
| 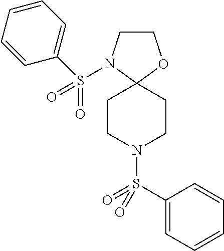 [SMILES: O=S(=O)(c1ccccc1)N1CCC2(CC1)OCCN2S(=O)(=O)c1ccccc1] | C0016 |
| 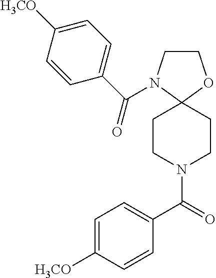 [SMILES: COc1ccc(C(=O)N2CCC3(CC2)OCCN3C(=O)c2ccc(OC)cc2)cc1] | C0017 |
| 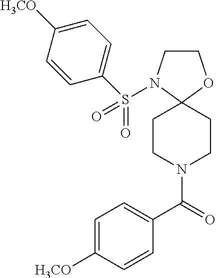 [SMILES: COc1ccc(C(=O)N2CCC3(CC2)OCCN3S(=O)(=O)c2ccc(OC)cc2)cc1] | C0018 |
| 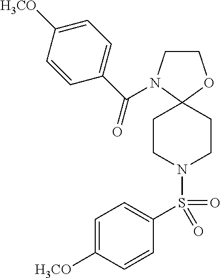 [SMILES: COc1ccc(C(=O)N2CCOC23CCN(S(=O)(=O)c2ccc(OC)cc2)CC3)cc1] | C0019 |
| 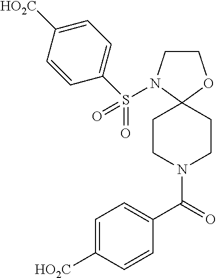 [SMILES: O=C(O)c1ccc(C(=O)N2CCC3(CC2)OCCN3S(=O)(=O)c2ccc(C(=O)O)cc2)cc1] | C0021 |
| 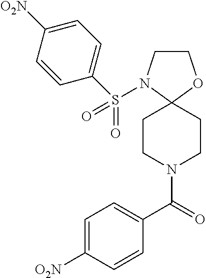 [SMILES: O=C(c1ccc([N+](=O)[O-])cc1)N1CCC2(CC1)OCCN2S(=O)(=O)c1ccc([N+](=O)[O-])cc1] | C0022 |
| 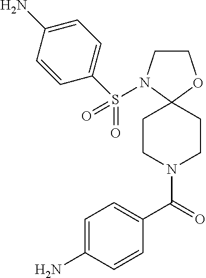 [SMILES: Nc1ccc(C(=O)N2CCC3(CC2)OCCN3S(=O)(=O)c2ccc(N)cc2)cc1] | C0023 |
| 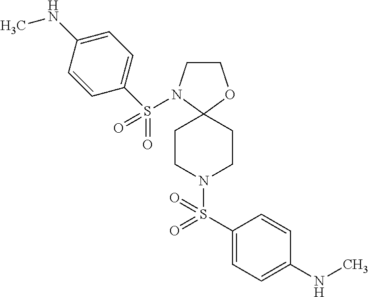 | C0024 |
| 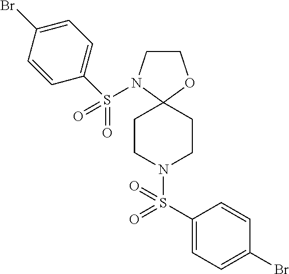 [SMILES: O=S(=O)(c1ccc(Br)cc1)N1CCC2(CC1)OCCN2S(=O)(=O)c1ccc(Br)cc1] | C0025 |
| 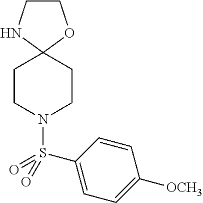 [SMILES: COc1ccc(S(=O)(=O)N2CCC3(CC2)NCCO3)cc1] | C0026 |
| 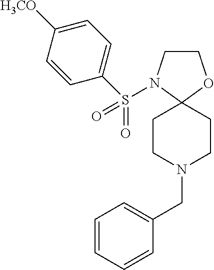 [SMILES: COc1ccc(S(=O)(=O)N2CCOC23CCN(Cc2ccccc2)CC3)cc1] | C0027-1 |
| 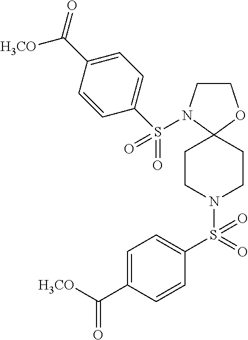 [SMILES: COC(=O)c1ccc(S(=O)(=O)N2CCC3(CC2)OCCN3S(=O)(=O)c2ccc(C(=O)OC)cc2)cc1] | C0028 |
| 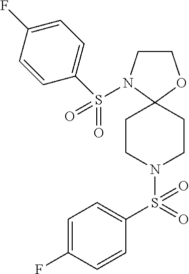 [SMILES: O=S(=O)(c1ccc(F)cc1)N1CCC2(CC1)OCCN2S(=O)(=O)c1ccc(F)cc1] | C0029 |
| 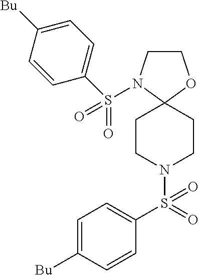 | C0030 |
| 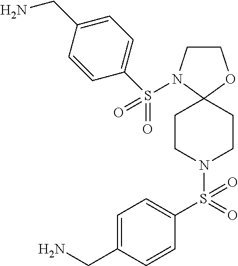 [SMILES: NCc1ccc(S(=O)(=O)N2CCC3(CC2)OCCN3S(=O)(=O)c2ccc(CN)cc2)cc1] | C0031 |
| 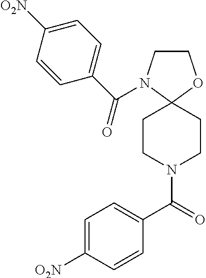 [SMILES: O=C(c1ccc([N+](=O)[O-])cc1)N1CCC2(CC1)OCCN2C(=O)c1ccc([N+](=O)[O-])cc1] | C0032 |
| 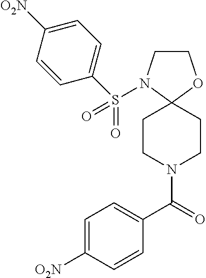 [SMILES: O=C(c1ccc([N+](=O)[O-])cc1)N1CCC2(CC1)OCCN2S(=O)(=O)c1ccc([N+](=O)[O-])cc1] | C0033 |
| 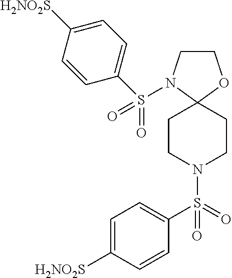 [SMILES: NS(=O)(=O)c1ccc(S(=O)(=O)N2CCC3(CC2)OCCN3S(=O)(=O)c2ccc(S(N)(=O)=O)cc2)cc1] | C0034 |
| 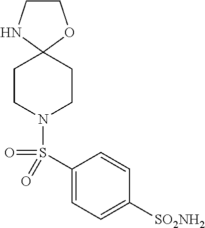 [SMILES: NS(=O)(=O)c1ccc(S(=O)(=O)N2CCC3(CC2)NCCO3)cc1] | C0034-3 |
| 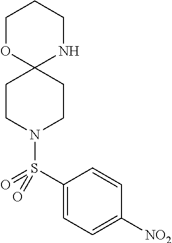 [SMILES: O=[N+]([O-])c1ccc(S(=O)(=O)N2CCC3(CC2)NCCCO3)cc1] | C0037-2 |
| 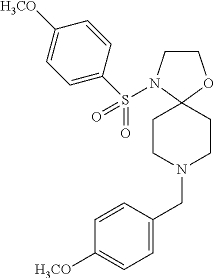 [SMILES: COc1ccc(CN2CCC3(CC2)OCCN3S(=O)(=O)c2ccc(OC)cc2)cc1] | C0038 |
| 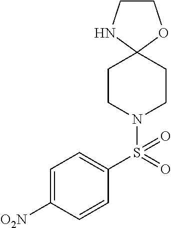 [SMILES: O=[N+]([O-])c1ccc(S(=O)(=O)N2CCC3(CC2)NCCO3)cc1] | C0040 |
| 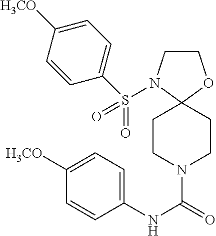 [SMILES: COc1ccc(NC(=O)N2CCC3(CC2)OCCN3S(=O)(=O)c2ccc(OC)cc2)cc1] | C0041 |
| 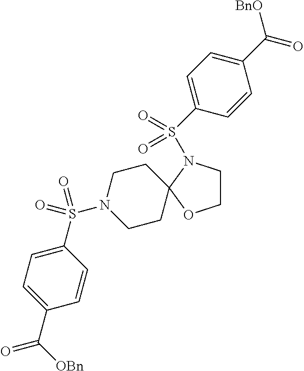 [SMILES: O=C(OCc1ccccc1)c1ccc(S(=O)(=O)N2CCC3(CC2)OCCN3S(=O)(=O)c2ccc(C(=O)OCc3ccccc3)cc2)cc1] | C0042 |
| 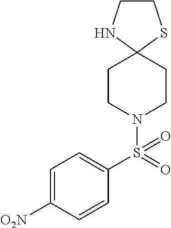 [SMILES: O=[N+]([O-])c1ccc(S(=O)(=O)N2CCC3(CC2)NCCS3)cc1] | C0044 |
| 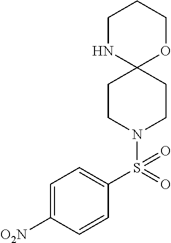 [SMILES: O=[N+]([O-])c1ccc(S(=O)(=O)N2CCC3(CC2)NCCCO3)cc1] | C0045 |
| 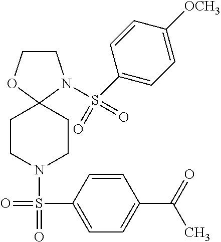 [SMILES: COc1ccc(S(=O)(=O)N2CCOC23CCN(S(=O)(=O)c2ccc(C(C)=O)cc2)CC3)cc1] | C0047 |
| 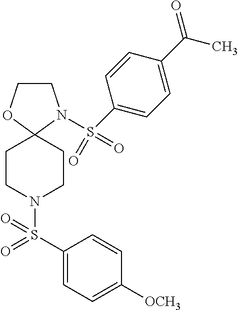 [SMILES: COc1ccc(S(=O)(=O)N2CCC3(CC2)OCCN3S(=O)(=O)c2ccc(C(C)=O)cc2)cc1] | C0048 |
| 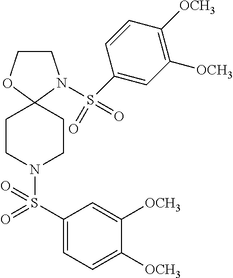 [SMILES: COc1ccc(S(=O)(=O)N2CCC3(CC2)OCCN3S(=O)(=O)c2ccc(OC)c(OC)c2)cc1OC] | C0049 |
| 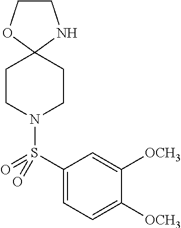 [SMILES: COc1ccc(S(=O)(=O)N2CCC3(CC2)NCCO3)cc1OC] | C0049-2 |
| 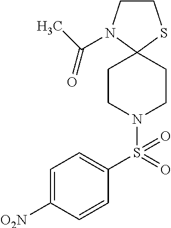 [SMILES: CC(=O)N1CCSC12CCN(S(=O)(=O)c1ccc([N+](=O)[O-])cc1)CC2] | C0050 |
| 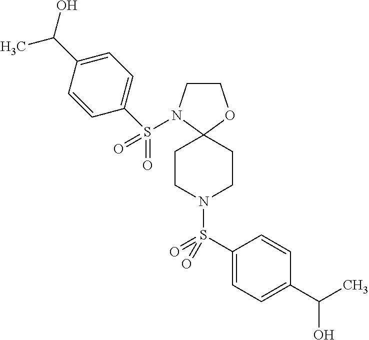 [SMILES: CC(O)c1ccc(S(=O)(=O)N2CCC3(CC2)OCCN3S(=O)(=O)c2ccc(C(C)O)cc2)cc1] | C0051 |
| 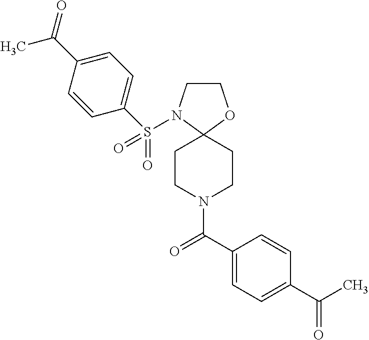 [SMILES: CC(=O)c1ccc(C(=O)N2CCC3(CC2)OCCN3S(=O)(=O)c2ccc(C(C)=O)cc2)cc1] | C0052 |
| 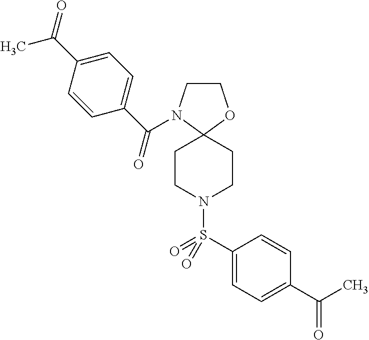 [SMILES: CC(=O)c1ccc(C(=O)N2CCOC23CCN(S(=O)(=O)c2ccc(C(C)=O)cc2)CC3)cc1] | C0053 |
| 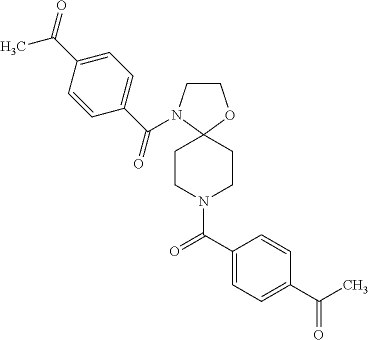 [SMILES: CC(=O)c1ccc(C(=O)N2CCC3(CC2)OCCN3C(=O)c2ccc(C(C)=O)cc2)cc1] | C0054 |
| 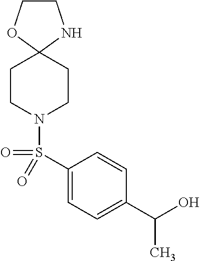 [SMILES: CC(O)c1ccc(S(=O)(=O)N2CCC3(CC2)NCCO3)cc1] | C0055-4 |
| 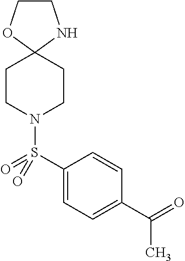 [SMILES: CC(=O)c1ccc(S(=O)(=O)N2CCC3(CC2)NCCO3)cc1] | C0055 |
| 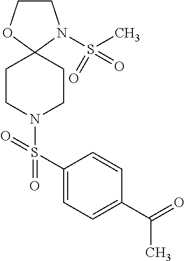 [SMILES: CC(=O)c1ccc(S(=O)(=O)N2CCC3(CC2)OCCN3S(C)(=O)=O)cc1] | C0056 |
| 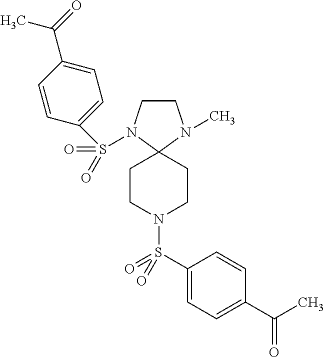 [SMILES: CC(=O)c1ccc(S(=O)(=O)N2CCC3(CC2)N(C)CCN3S(=O)(=O)c2ccc(C(C)=O)cc2)cc1] | C0057 |
| 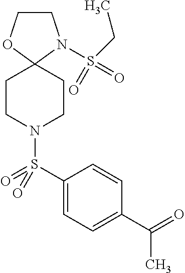 [SMILES: CCS(=O)(=O)N1CCOC12CCN(S(=O)(=O)c1ccc(C(C)=O)cc1)CC2] | C0058 |
| 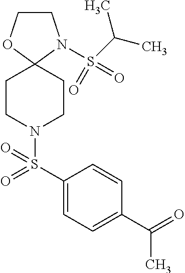 [SMILES: CC(=O)c1ccc(S(=O)(=O)N2CCC3(CC2)OCCN3S(=O)(=O)C(C)C)cc1] | C0059 |
| 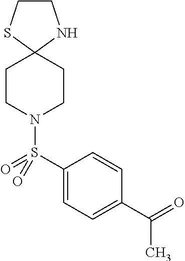 [SMILES: CC(=O)c1ccc(S(=O)(=O)N2CCC3(CC2)NCCS3)cc1] | C0060 |
| 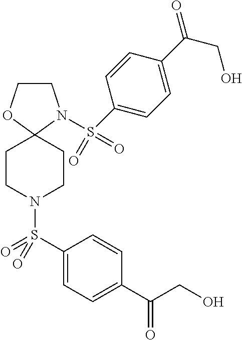 [SMILES: O=C(CO)c1ccc(S(=O)(=O)N2CCC3(CC2)OCCN3S(=O)(=O)c2ccc(C(=O)CO)cc2)cc1] | C0061 |
| 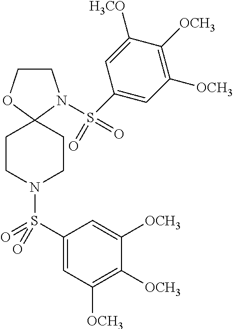 [SMILES: COc1cc(S(=O)(=O)N2CCC3(CC2)OCCN3S(=O)(=O)c2cc(OC)c(OC)c(OC)c2)cc(OC)c1OC] | C0062 |
| 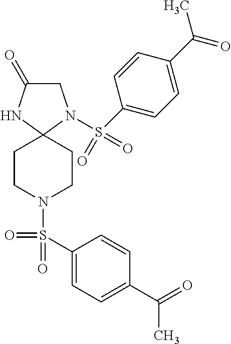 [SMILES: CC(=O)c1ccc(S(=O)(=O)N2CCC3(CC2)NC(=O)CN3S(=O)(=O)c2ccc(C(C)=O)cc2)cc1] | C0064 |
| 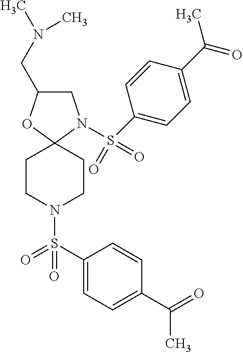 [SMILES: CC(=O)c1ccc(S(=O)(=O)N2CCC3(CC2)OC(CN(C)C)CN3S(=O)(=O)c2ccc(C(C)=O)cc2)cc1] | C0065 |
| 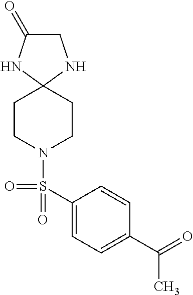 [SMILES: CC(=O)c1ccc(S(=O)(=O)N2CCC3(CC2)NCC(=O)N3)cc1] | C0066 |
| 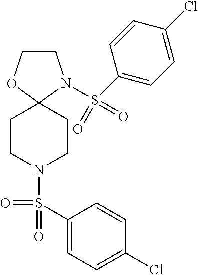 [SMILES: O=S(=O)(c1ccc(Cl)cc1)N1CCC2(CC1)OCCN2S(=O)(=O)c1ccc(Cl)cc1] | C0067 |
| 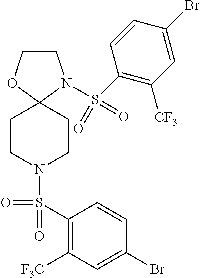 [SMILES: O=S(=O)(c1ccc(Br)cc1C(F)(F)F)N1CCC2(CC1)OCCN2S(=O)(=O)c1ccc(Br)cc1C(F)(F)F] | C0068 |
| 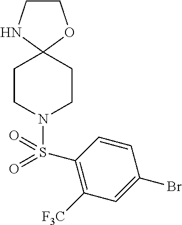 [SMILES: O=S(=O)(c1ccc(Br)cc1C(F)(F)F)N1CCC2(CC1)NCCO2] | C0068-2 |
| 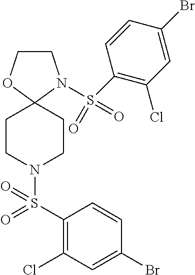 [SMILES: O=S(=O)(c1ccc(Br)cc1Cl)N1CCC2(CC1)OCCN2S(=O)(=O)c1ccc(Br)cc1Cl] | C0069 |
| 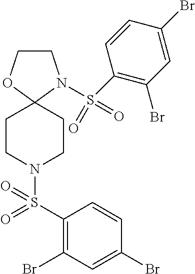 [SMILES: O=S(=O)(c1ccc(Br)cc1Br)N1CCC2(CC1)OCCN2S(=O)(=O)c1ccc(Br)cc1Br] | C0070 |
| 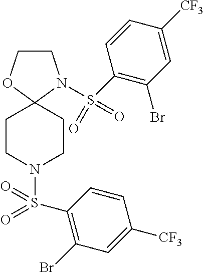 [SMILES: O=S(=O)(c1ccc(C(F)(F)F)cc1Br)N1CCC2(CC1)OCCN2S(=O)(=O)c1ccc(C(F)(F)F)cc1Br] | C0071 |
| 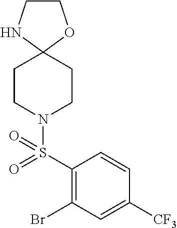 [SMILES: O=S(=O)(c1ccc(C(F)(F)F)cc1Br)N1CCC2(CC1)NCCO2] | C0071-2 |
| 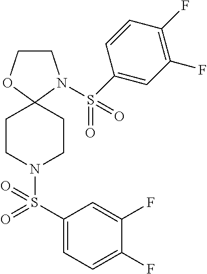 [SMILES: O=S(=O)(c1ccc(F)c(F)c1)N1CCC2(CC1)OCCN2S(=O)(=O)c1ccc(F)c(F)c1] | C0072 |
| 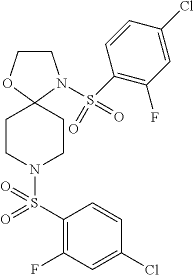 [SMILES: O=S(=O)(c1ccc(Cl)cc1F)N1CCC2(CC1)OCCN2S(=O)(=O)c1ccc(Cl)cc1F] | C0073 |
| 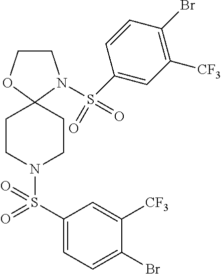 [SMILES: O=S(=O)(c1ccc(Br)c(C(F)(F)F)c1)N1CCC2(CC1)OCCN2S(=O)(=O)c1ccc(Br)c(C(F)(F)F)c1] | C0077 |
| 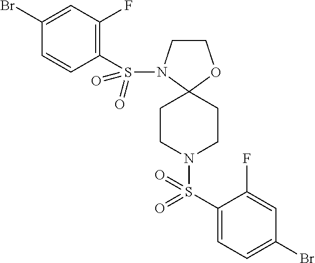 [SMILES: O=S(=O)(c1ccc(Br)cc1F)N1CCC2(CC1)OCCN2S(=O)(=O)c1ccc(Br)cc1F] | C0078 |
| 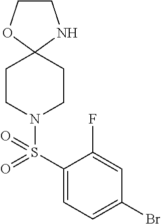 [SMILES: O=S(=O)(c1ccc(Br)cc1F)N1CCC2(CC1)NCCO2] | C0078-2 |
| 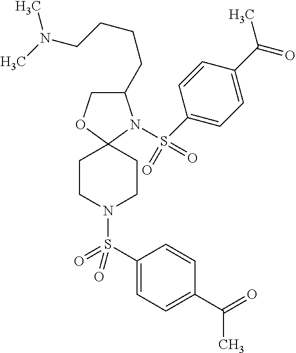 [SMILES: CC(=O)c1ccc(S(=O)(=O)N2CCC3(CC2)OCC(CCCCN(C)C)N3S(=O)(=O)c2ccc(C(C)=O)cc2)cc1] | C0080 |
| 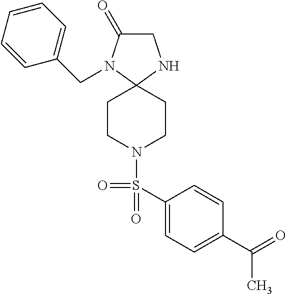 [SMILES: CC(=O)c1ccc(S(=O)(=O)N2CCC3(CC2)NCC(=O)N3Cc2ccccc2)cc1] | C0082M |
| 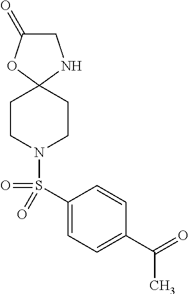 [SMILES: CC(=O)c1ccc(S(=O)(=O)N2CCC3(CC2)NCC(=O)O3)cc1] | C0083M |
| 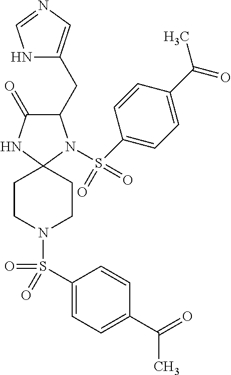 [SMILES: CC(=O)c1ccc(S(=O)(=O)N2CCC3(CC2)NC(=O)C(Cc2cnc[nH]2)N3S(=O)(=O)c2ccc(C(C)=O)cc2)cc1] | C0084M |
| 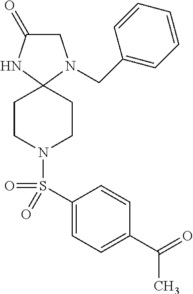 [SMILES: CC(=O)c1ccc(S(=O)(=O)N2CCC3(CC2)NC(=O)CN3Cc2ccccc2)cc1] | C0085M |
| 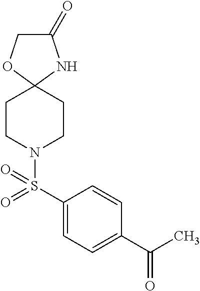 [SMILES: CC(=O)c1ccc(S(=O)(=O)N2CCC3(CC2)NC(=O)CO3)cc1] | C0087M |
| 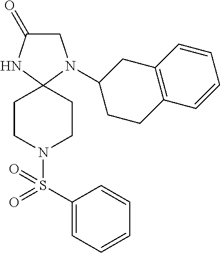 [SMILES: O=C1CN(C2CCc3ccccc3C2)C2(CCN(S(=O)(=O)c3ccccc3)CC2)N1] | C0136M/ (P5) |
| 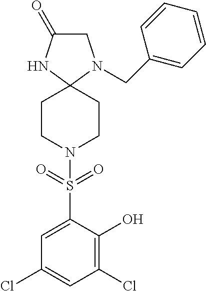 [SMILES: O=C1CN(Cc2ccccc2)C2(CCN(S(=O)(=O)c3cc(Cl)cc(Cl)c3O)CC2)N1] | C0138M |
| 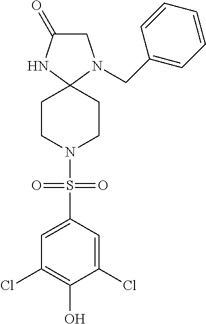 [SMILES: O=C1CN(Cc2ccccc2)C2(CCN(S(=O)(=O)c3cc(Cl)c(O)c(Cl)c3)CC2)N1] | C0139M |
| 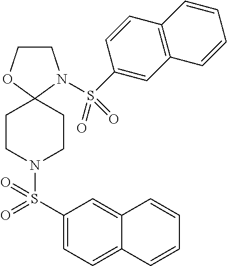 [SMILES: O=S(=O)(c1ccc2ccccc2c1)N1CCC2(CC1)OCCN2S(=O)(=O)c1ccc2ccccc2c1] | C0140M |
| 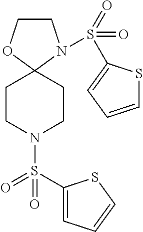 [SMILES: O=S(=O)(c1cccs1)N1CCC2(CC1)OCCN2S(=O)(=O)c1cccs1] | C0141M |
| 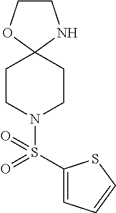 [SMILES: O=S(=O)(c1cccs1)N1CCC2(CC1)NCCO2] | C0141M-2 |
| 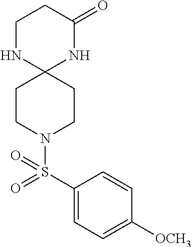 [SMILES: COc1ccc(S(=O)(=O)N2CCC3(CC2)NCCC(=O)N3)cc1] | C0142M |
| 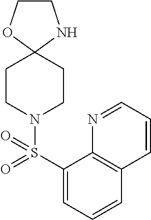 [SMILES: O=S(=O)(c1cccc2cccnc12)N1CCC2(CC1)NCCO2] | C0143M-2 |
| 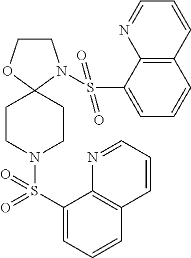 [SMILES: O=S(=O)(c1cccc2cccnc12)N1CCC2(CC1)OCCN2S(=O)(=O)c1cccc2cccnc12] | C0143M |
| 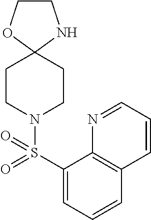 [SMILES: O=S(=O)(c1cccc2cccnc12)N1CCC2(CC1)NCCO2] | C0143M-2 |
| 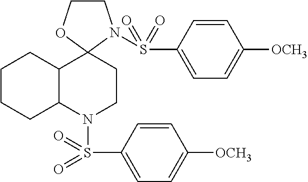 [SMILES: COc1ccc(S(=O)(=O)N2CCC3(OCCN3S(=O)(=O)c3ccc(OC)cc3)C3CCCCC32)cc1] | C0144M |
| 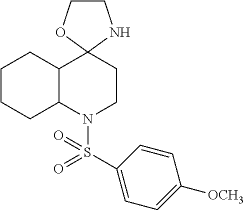 [SMILES: COc1ccc(S(=O)(=O)N2CCC3(NCCO3)C3CCCCC32)cc1] | C0144M-2 |
| 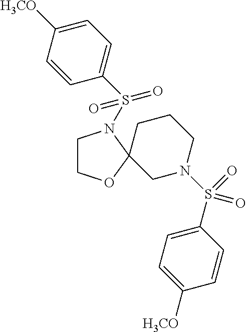 [SMILES: COc1ccc(S(=O)(=O)N2CCCC3(C2)OCCN3S(=O)(=O)c2ccc(OC)cc2)cc1] | C0145M |
| 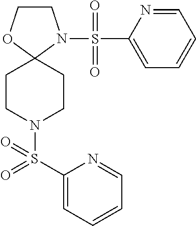 [SMILES: O=S(=O)(c1ccccn1)N1CCC2(CC1)OCCN2S(=O)(=O)c1ccccn1] | C0146M |
| 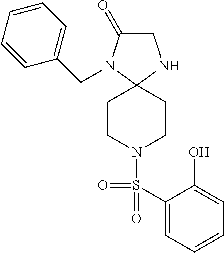 [SMILES: O=C1CNC2(CCN(S(=O)(=O)c3ccccc3O)CC2)N1Cc1ccccc1] | C0147M A2 |
| 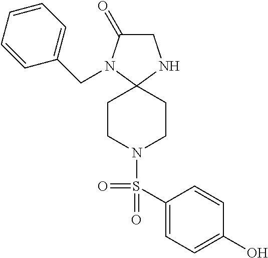 [SMILES: O=C1CNC2(CCN(S(=O)(=O)c3ccc(O)cc3)CC2)N1Cc1ccccc1] | C0148M |
| 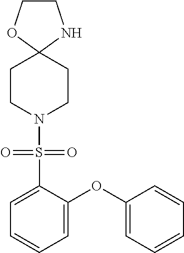 [SMILES: O=S(=O)(c1ccccc1Oc1ccccc1)N1CCC2(CC1)NCCO2] | C0149M-2 |
| 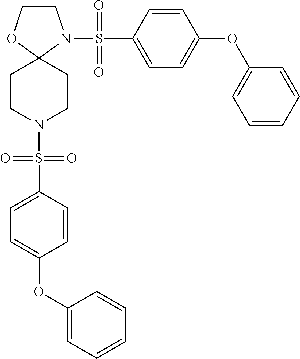 [SMILES: O=S(=O)(c1ccc(Oc2ccccc2)cc1)N1CCC2(CC1)OCCN2S(=O)(=O)c1ccc(Oc2ccccc2)cc1] | C0149M |
| 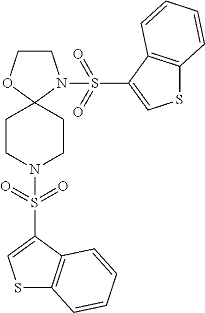 [SMILES: O=S(=O)(c1csc2ccccc12)N1CCC2(CC1)OCCN2S(=O)(=O)c1csc2ccccc12] | C0150M |
| 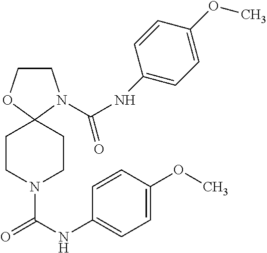 [SMILES: COc1ccc(NC(=O)N2CCC3(CC2)OCCN3C(=O)Nc2ccc(OC)cc2)cc1] | C0151M |
| 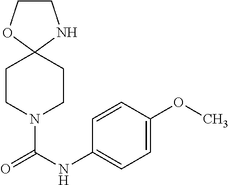 [SMILES: COc1ccc(NC(=O)N2CCC3(CC2)NCCO3)cc1] | C0151M-2 |
| 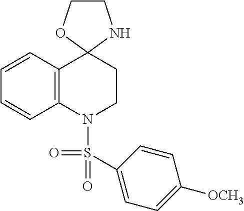 [SMILES: COc1ccc(S(=O)(=O)N2CCC3(NCCO3)c3ccccc32)cc1] | C0152M-4 |

Table of Series C-2 Compounds

| | |
|---|---|
| 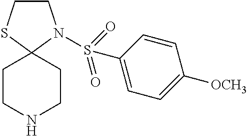 [SMILES: COc1ccc(S(=O)(=O)N2CCSC23CCNCC3)cc1] | S-C0027 |
| 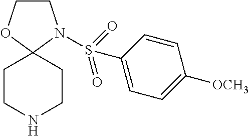 [SMILES: COc1ccc(S(=O)(=O)N2CCOC23CCNCC3)cc1] | C0027 |
| 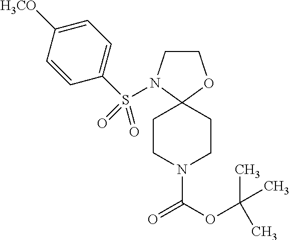 [SMILES: COc1ccc(S(=O)(=O)N2CCOC23CCN(C(=O)OC(C)(C)C)CC3)cc1] | C0043 |
| 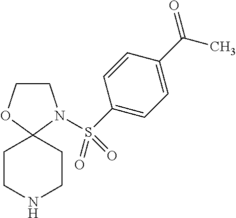 [SMILES: CC(=O)c1ccc(S(=O)(=O)N2CCOC23CCNCC3)cc1] | C0046 |
| 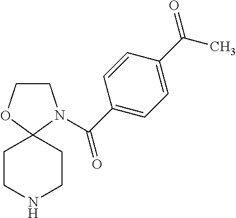 [SMILES: CC(=O)c1ccc(C(=O)N2CCOC23CCNCC3)cc1] | C0053-3 |
| 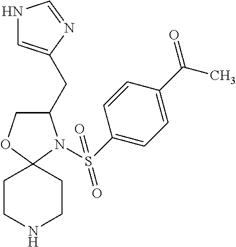 [SMILES: CC(=O)c1ccc(S(=O)(=O)N2C(Cc3c[nH]cn3)COC23CCNCC3)cc1] | C0079M-7 |
| 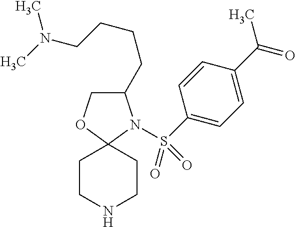 [SMILES: CC(=O)c1ccc(S(=O)(=O)N2C(CCCCN(C)C)COC23CCNCC3)cc1] | C0080M-6 |
| 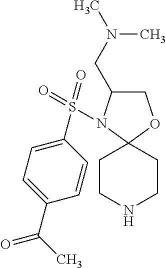 [SMILES: CC(=O)c1ccc(S(=O)(=O)N2C(CN(C)C)COC23CCNCC3)cc1] | C0081M-7 |
| 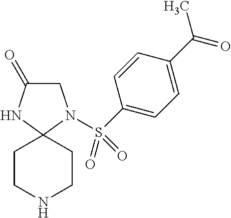 [SMILES: CC(=O)c1ccc(S(=O)(=O)N2CC(=O)NC23CCNCC3)cc1] | C0086M |
| 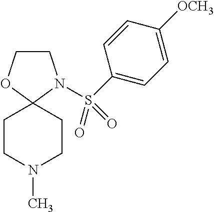 [SMILES: COc1ccc(S(=O)(=O)N2CCOC23CCN(C)CC3)cc1] | C0088M |
| 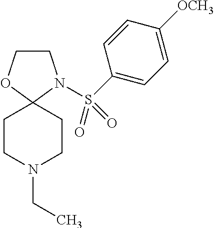 [SMILES: CCN1CCC2(CC1)OCCN2S(=O)(=O)c1ccc(OC)cc1] | C0089M |
| 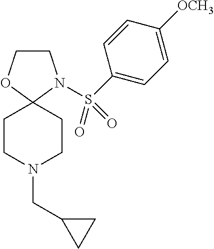 [SMILES: COc1ccc(S(=O)(=O)N2CCOC23CCN(CC2CC2)CC3)cc1] | C0090M |
| 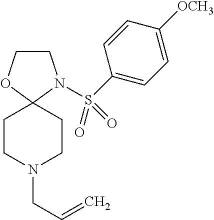 [SMILES: C=CCN1CCC2(CC1)OCCN2S(=O)(=O)c1ccc(OC)cc1] | C0091M |
| 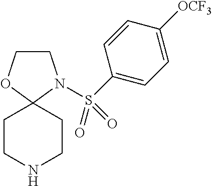 [SMILES: O=S(=O)(c1ccc(OC(F)(F)F)cc1)N1CCOC12CCNCC2] | C0092M |
| 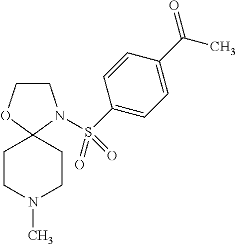 [SMILES: CC(=O)c1ccc(S(=O)(=O)N2CCOC23CCN(C)CC3)cc1] | C0093M |
| 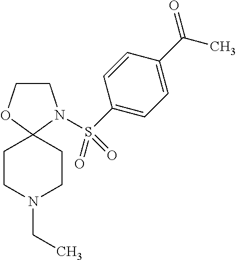 [SMILES: CCN1CCC2(CC1)OCCN2S(=O)(=O)c1ccc(C(C)=O)cc1] | C0094M |
| 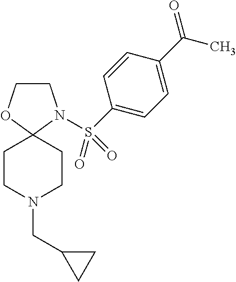 [SMILES: CC(=O)c1ccc(S(=O)(=O)N2CCOC23CCN(CC2CC2)CC3)cc1] | C0095M |
| 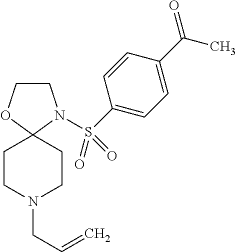 [SMILES: C=CCN1CCC2(CC1)OCCN2S(=O)(=O)c1ccc(C(C)=O)cc1] | C0096M |
| 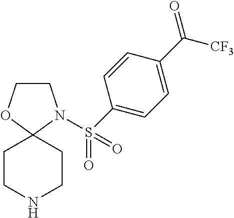 [SMILES: O=C(c1ccc(S(=O)(=O)N2CCOC23CCNCC3)cc1)C(F)(F)F] | C0097M |
| 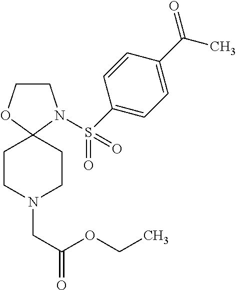 [SMILES: CCOC(=O)CN1CCC2(CC1)OCCN2S(=O)(=O)c1ccc(C(C)=O)cc1] | C0099M |
| 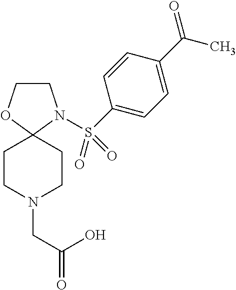 [SMILES: CC(=O)c1ccc(S(=O)(=O)N2CCOC23CCN(CC(=O)O)CC3)cc1] | C0100M |
| 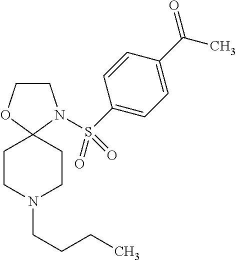 [SMILES: CCCCN1CCC2(CC1)OCCN2S(=O)(=O)c1ccc(C(C)=O)cc1] | C0101M |
| 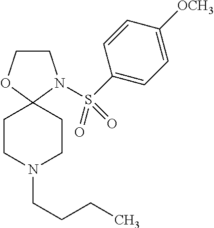 [SMILES: CCCCN1CCC2(CC1)OCCN2S(=O)(=O)c1ccc(OC)cc1] | C0102M |
| 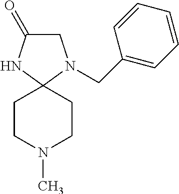 [SMILES: CN1CCC2(CC1)NC(=O)CN2Cc1ccccc1] | C0104M |
| 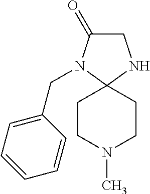 [SMILES: CN1CCC2(CC1)NCC(=O)N2Cc1ccccc1] | C0105M |
| 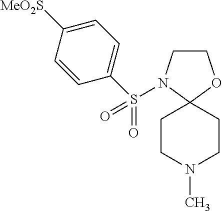 [SMILES: CN1CCC2(CC1)OCCN2S(=O)(=O)c1ccc(S(C)(=O)=O)cc1] | C0106M |
| 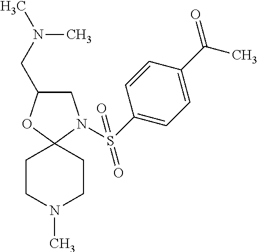 [SMILES: CC(=O)c1ccc(S(=O)(=O)N2CC(CN(C)C)OC23CCN(C)CC3)cc1] | C0108M |
| 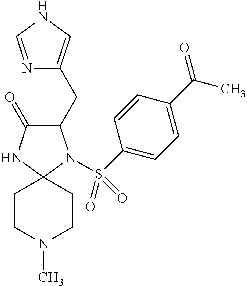 [SMILES: CC(=O)c1ccc(S(=O)(=O)N2C(Cc3c[nH]cn3)C(=O)NC23CCN(C)CC3)cc1] | C0109M |
| 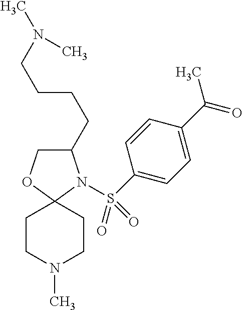 [SMILES: CC(=O)c1ccc(S(=O)(=O)N2C(CCCCN(C)C)COC23CCN(C)CC3)cc1] | C0111M |
| 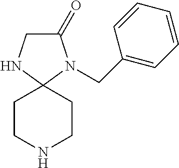 [SMILES: O=C1CNC2(CCNCC2)N1Cc1ccccc1] | C0114M |
| 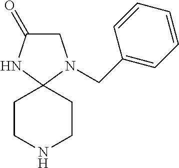 [SMILES: O=C1CN(Cc2ccccc2)C2(CCNCC2)N1] | C0115M |
| 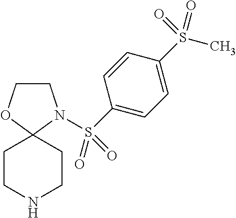 [SMILES: CS(=O)(=O)c1ccc(S(=O)(=O)N2CCOC23CCNCC3)cc1] | C0116M |
| 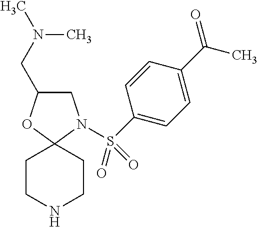 [SMILES: CC(=O)c1ccc(S(=O)(=O)N2CC(CN(C)C)OC23CCNCC3)cc1] | C0118M |
| 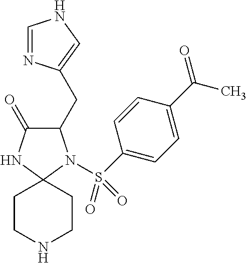 [SMILES: CC(=O)c1ccc(S(=O)(=O)N2C(Cc3c[nH]cn3)C(=O)NC23CCNCC3)cc1] | C0119M |
| 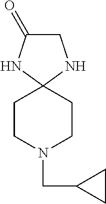 [SMILES: O=C1CNC2(CCN(CC3CC3)CC2)N1] | C0123M |
| 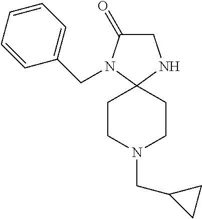 [SMILES: O=C1CNC2(CCN(CC3CC3)CC2)N1Cc1ccccc1] | C0142M |
| 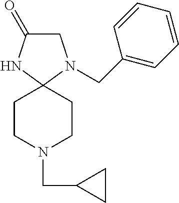 [SMILES: O=C1CN(Cc2ccccc2)C2(CCN(CC3CC3)CC2)N1] | C0125M |
| 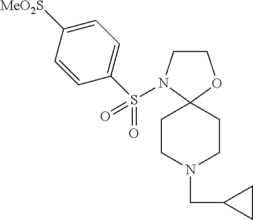 [SMILES: CS(=O)(=O)c1ccc(S(=O)(=O)N2CCOC23CCN(CC2CC2)CC3)cc1] | C0126M |
| 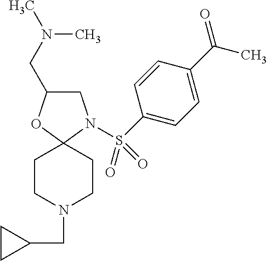 [SMILES: CC(=O)c1ccc(S(=O)(=O)N2CC(CN(C)C)OC23CCN(CC2CC2)CC3)cc1] | C0128M |
| 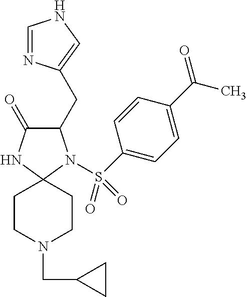 [SMILES: CC(=O)c1ccc(S(=O)(=O)N2C(Cc3c[nH]cn3)C(=O)NC23CCN(CC2CC2)CC3)cc1] | C0129M |
| 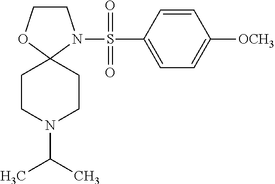 [SMILES: COc1ccc(S(=O)(=O)N2CCOC23CCN(C(C)C)CC3)cc1] | C0133M |
| 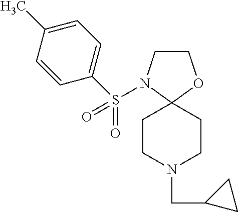 [SMILES: Cc1ccc(S(=O)(=O)N2CCOC23CCN(CC2CC2)CC3)cc1] | C0134M |
| 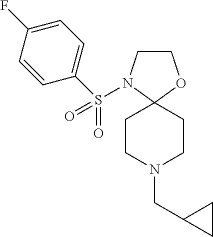 [SMILES: O=S(=O)(c1ccc(F)cc1)N1CCOC12CCN(CC1CC1)CC2] | F-C0134 |
| 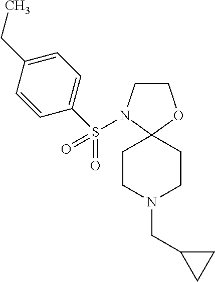 [SMILES: CCc1ccc(S(=O)(=O)N2CCOC23CCN(CC2CC2)CC3)cc1] | C0135M |
| 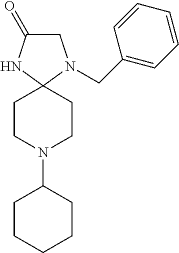 [SMILES: O=C1CN(Cc2ccccc2)C2(CCN(C3CCCCC3)CC2)N1] | C0137M P7 |
| 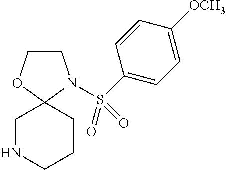 [SMILES: COc1ccc(S(=O)(=O)N2CCOC23CCCNC3)cc1] | C0145M-3 |
| 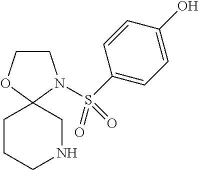 [SMILES: O=S(=O)(c1ccc(O)cc1)N1CCOC12CCCNC2] | C0153M-3 |
| 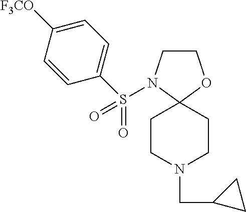 [SMILES: O=S(=O)(c1ccc(OC(F)(F)F)cc1)N1CCOC12CCN(CC1CC1)CC2] | Compound 4 |
| 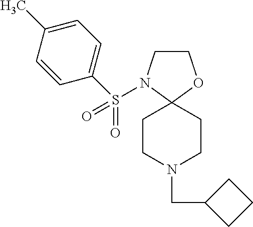 [SMILES: Cc1ccc(S(=O)(=O)N2CCOC23CCN(CC2CCC2)CC3)cc1] | Compound 9 |
| 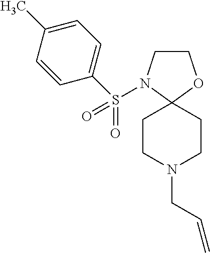 [SMILES: C=CCN1CCC2(CC1)OCCN2S(=O)(=O)c1ccc(C)cc1] | Compound 10 |

Table of Series D Compounds

| | |
|---|---|
| 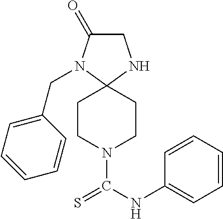 [SMILES: O=C1CNC2(CCN(C(=S)Nc3ccccc3)CC2)N1Cc1ccccc1] | Compound A |
| 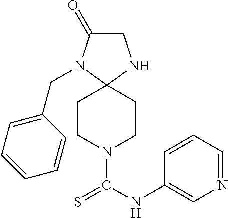 [SMILES: O=C1CNC2(CCN(C(=S)Nc3cccnc3)CC2)N1Cc1ccccc1] | Compound B |
| 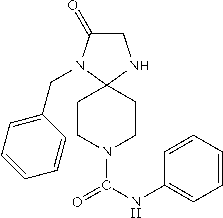 [SMILES: O=C(Nc1ccccc1)N1CCC2(CC1)NCC(=O)N2Cc1ccccc1] | Compound C |

Preparation of Series C-2 Compounds 4, 9 and 10 and those of Compounds A, B and of Series D are described in U.S. Pat. No. 10,017,736, whose disclosures are incorporated by reference.

### EXAMPLE 1

### FITC-NLX-Based FLNA Screening Assay

A. Streptavidin-Coated 96-Well Plates

Streptavidin-coated 96-well plates (Reacti-Bind™ NeutrAvidin™ High binding capacity coated 96-well plate, Pierce-ENDOGEN) are washed three times with 200 ml of 50 mM Tris HCl, pH 7.4 according to the manufacturer's recommendation.

B. N-Biotinylated FLNA Pentapeptide

The biotinylated FLNA peptide (0.5 mg/plate) is dissolved in 50 ml DMSO and then added to 4450 ml of 50 mM Tris HCl, pH 7.4, containing 100 mM NaCl and protease inhibitors (binding medium) as well as 500 ml superblock in PBS (Pierce-ENDOGEN) [final concentration for DMSO: 1%].

C. Coupling of the Biotinylated FLNA Pentapeptide to Streptavidin-Coated Plate

The washed streptavidin-coated plates are contacted with 5 mg/well of the biotinylated FLNA pentapeptide of positions 2561-2565 (100 ml) for 1 hour (incubated) with constant shaking at 25° C. [50 ml of the peptide solution from B+50 ml binding medium, final concentration for DMSO: 0.5%]. At the end of the incubation, the plate is washed three times with 200 ml of ice-cold 50 mM Tris HCl, pH 7.4.

D. Binding of FITC-Tagged Naloxone [FITC-NLX] to Biotinylated FLNA Peptide

Biotinylated FLNA pentapeptide-coated streptavidin plates are incubated with 10 nM fluorescein isothiocyanate-labeled naloxone (FITC-NLX; Invitrogen) in binding medium (50 mM Tris HCl, pH 7.4 containing 100 mM NaCl and protease inhibitors) for 30 minutes at 30° C. with constant shaking. The final assay volume is 100 ml. At the end of incubation, the plate is washed twice with 100 ml of ice-cold 50 mM Tris, pH 7.4. The signal, bound-FITC-NLX is detected using a DTX-880 multi-mode plate reader (Beckman).

E. Screening of Medicinal Chemistry Analogs

The compounds are first individually dissolved in 25% DMSO containing 50 mM Tris HCl, pH 7.4, to a final concentration of 1 mM (assisted by sonication when necessary) and then plated into 96-well compound plates. To screen the medicinal chemistry analogs (new compounds), each compound solution (1 ml) is added to the biotinylated FLNA pentapeptide coated streptavidin plate with 50 ml/well of binding medium followed immediately with addition of 50 ml of FITC-NLX (total assay volume/well is 100 ml). The final screening concentration for each new compound is initially 10 mM.

Each screening plate includes vehicle control (total binding) as well as naloxone (NLX) and/or naltrexone (NTX) as positive controls. Compounds are tested in triplicate or quadruplicate. Percent inhibition of FITC-NLX binding for each compound is calculated [(Total FITC-NLX bound in vehicle−FITC-NLX bound with compound)/Total FITC-NLX bound in vehicle]×100%]. To assess the efficacies and potencies of the selected compounds, compounds that achieve approximately 60-70% inhibition at 10 mM are screened further at 1 and 0.1 mM concentrations.

The results of this screening assay are shown in the tables below.

FLNA Peptide Binding Assays

C-Series-1 Compounds

| | Concentration of FLNA-binding Compound | | |
|---|---|---|---|
| FLNA-binding | 0.01 μM | 0.1 μM | 1 μM |
| Compound | Percent Binding Inhibition | | |
| Naloxone | 39.87% | 46.29% | 50.91% |
| Control | | | |
| Average | | | |
| 7866 | 38.5% | 47.9% | 53.4% |
| C0001 | 34.8% | 42.9% | 51.3% |
| C0002 | 38.4% | 45.6% | 42.8% |
| C0003 | 38.3% | 45.3% | 48.8% |
| C0004 | 37.6% | 42.3% | 44.7% |
| C0005 | 35.2% | 44.5% | 51.5% |
| C0006 | 41.6% | 46.8% | 51.8% |
| C0007 | 40.5% | 46.3% | 48.9% |
| C0008 | 42.2% | 52.3% | 54.4% |
| C0009 | 41.7% | 49.0% | 53.9% |
| C0010 | 39.8% | 42.7% | 47.1% |
| C0011 | 37.6% | 41.4% | 46.0% |
| C0012 | 26.3% | 39.5% | 46.4% |
| C0013 | 39.6% | 42.4% | 49.1% |
| C0014 | 29.5% | 38.8% | 40.0% |
| C0015 | 31.2% | 40.6% | 45.5% |
| C0016 | 38.3% | 43.8% | 49.1% |
| C0017 | 28.9% | 35.4% | 40.7% |
| C0018 | 42.3% | 45.9% | 53.4% |
| C0019 | 30.1% | 38.2% | 43.6% |
| C0021 | 34.0% | 38.4% | 40.6% |
| C0022 | 34.5% | 37.6% | 43.9% |
| C0023 | 35.9% | 41.7% | 47.2% |
| C0024 | 37.9% | 46.4% | 50.4% |
| C0025 | 37.2% | 41.4% | 45.1% |
| C0028 | 32.2% | 36.6% | 43.3% |
| C0029 | 38.6% | 43.2% | 50.5% |
| C0030 | 37.4% | 45.4% | 56.0% |
| C0032 | 41.5% | 50.5% | 55.3% |
| C0033 | 43.9% | 48.4% | 51.3% |
| C0034 | 29.6% | 38.3% | 44.8% |
| C0038 | 31.7% | 36.0% | 43.5% |
| C0041 | 38.3% | 47.0% | 51.2% |
| C0042 | 42.4% | 49.7% | 56.1% |
| C0047 | 30.8% | 35.2% | 41.4% |
| C0048 | 28.5% | 38.9% | 45.9% |
| C0049 | 25.3% | 27.9% | 30.3% |
| C0051 | 27.0% | 30.4% | 36.4% |
| C0052 | 28.0% | 35.6% | 40.8% |
| C0053 | 28.9% | 33.8% | 39.3% |
| C0054 | 32.9% | 39.4% | 43.3% |
| C0057 | ND* | ND | ND |
| C0060 | 60.3% | 64.0% | 68.0% |
| C0061 | ND | ND | ND |
| C0062 | 39.5% | 49.5% | 48.0% |
| C0064 | 37.3% | 44.4% | 49.2% |
| C0065 | 37.1% | 44.0% | 47.0% |
| C0067 | 31.3% | 39.7% | 45.0% |
| C0068 | 53.7% | 58.6% | 62.2% |
| C0069 | ND | ND | ND |
| C0070 | 42.6% | 50.6% | 53.6% |
| C0071 | 39.1% | 49.6% | 55.2% |
| C0072 | 28.4% | 37.4% | 44.0% |
| C0073 | ND | ND | ND |
| C0077 | 45.7% | 47.7% | 51.0% |
| C0078 | 46.6% | 48.0% | 50.5% |
| C0080M | 46.8% | 53.3% | 54.6% |
| C0084M | 47.2% | 53.7% | 55.9% |
| C0085M | 45.7% | 53.7% | 60.7% |
| C0138M | 53.0% | 52.0% | 59.5% |
| C0139M | 48.9% | 53.1% | 61.6% |
| C0140M | 42.3% | 49.2% | 54.4% |
| C0141M | 33.1% | 39.0% | 46.9% |
| C0143M | 45.3% | 48.4% | 57.8% |
| C0144M | 46.4% | 50.7% | 55.7% |
| C0145M | 45.1% | 53.7% | 58.3% |
| C0148M | 46.2% | 52.0% | 57.0% |
| C0149M | 48.5% | 52.3% | 62.0% |
| C0150M | 47.3% | 51.8% | 61.4% |
| C0151M | 48.3% | 51.7% | 58.7% |
| C0152M | ND | ND | ND |
| C0154M | ND | ND | ND |
| Naloxone | 39.87% | 46.29% | 50.91% |
| Control | | | |
| Average | | | |

*ND = Not Done.

C-Series-2 Compounds

| | Concentration of FLNA-binding Compound | | |
|---|---|---|---|
| FLNA-binding | 0.01 μM | 0.1 μM | 1 μM |
| Compound | Percent Binding Inhibition | | |
| Naloxone | 39.87 | 46.29% | 50.91 |
| Control | | | |
| Average | | | |
| C0011 | 37.6% | 41.4% | 46.0% |
| C0026 | 42.3% | 44.8% | 49.0% |
| C0027 | 50.8% | 61.2% | 63.8% |
| S-00027 | 39.1% | 46.5% | 53.6% |
| C0034-3 | 29.6% | 38.3% | 44.8% |
| C0037-2 | ND* | ND | ND |
| C0040 | 38.4% | 46.3% | 55.9% |
| C0043 | 43.9% | 51.3% | 58.0% |
| C0044 | 37.3% | 43.9% | 50.6% |
| C0045 | 39.1% | 48.9% | 53.7% |
| C0046 | 30.8% | 35.7% | 42.2% |
| C0050 | 26.7% | 34.5% | 36.4% |
| C0055 | 29.0% | 34.9% | 39.5% |
| C0056 | 33.7% | 38.9% | 41.4% |
| C0060 | 60.3% | 64.0% | 68.0% |
| C0086M | 37.9% | 48.1% | 53.4% |
| C0087M | 51.6% | 57.9% | 61.5% |
| C0088M | 40.1% | 52.4% | 56.1% |
| C0089M | 40.7% | 46.1% | 51.2% |
| C0090M | 42.5% | 52.5% | 55.8% |
| C0091M | 38.1% | 39.8% | 46.3% |
| C0093M | 44.8% | 49.9% | 53.5% |
| C0094M | 43.0% | 52.8% | 57.5% |
| C0095M | 40.1% | 46.6% | 50.5% |
| C0096M | 43.0% | 48.3% | 55.0% |
| C0099M | 46.9% | 53.3% | 56.0% |
| C0100M | 52.2% | 58.2% | 64.5% |
| C0101M | 50.5% | 56.4% | 59.0% |
| C0102M | 52.3% | 53.1% | 56.6% |
| C0104M | 51.4% | 54.1% | 55.2% |
| C0105M | 55.7% | 62.0% | 68.8% |
| C0106M | 45.8% | 55.6% | 58.9% |
| C0108M | 54.6% | 61.4% | 68.7% |
| C0114M | 57.1% | 63.2% | 66.7% |
| C0115M | 47.8% | 57.8% | 59.9% |
| C0116M | 53.9% | 60.0% | 62.9% |
| C0118M | 56.6% | 61.4% | 62.4% |
| C0119M | 41.6% | 55.5% | 60.0% |
| C0123M | 51.9% | 60.5% | 62.9% |
| C0124M | 47.7% | 52.2% | 58.7% |
| C0125M | 54.2% | 59.7% | 63.3% |
| C0126M | 50.7% | 55.4% | 67.3% |
| C0128M | 46.5% | 54.4% | 58.2% |
| C0133M | 47.8% | 54.9% | 58.5% |
| C0134M | 55.7% | 60.5% | 61.9% |
| F-00134 | 37.4% | 45.7% | 53.1% |
| C0135M | 53.9% | 55.1% | 62.3% |
| C0136M(P5) | 46.7% | 55.2% | 58.2% |
| C0137M(P7) | 42.4% | 49.9% | 61.2% |
| C0142M | 35.1% | 39.4% | 56.0% |
| C0143M | 45.3% | 48.4% | 57.8% |
| C0148M | 46.2% | 52.0% | 57.0% |
| C0149M | 48.5% | 52.3% | 62.0% |
| C0150M | 47.3% | 51.8% | 61.4% |
| C0151M | 48.3% | 51.7% | 58.7% |
| C0152M-4 | ND | ND | ND |
| C0153M-3 | ND | ND | ND |
| Naloxone | 39.87% | 46.29% | 50.91% |
| Control | | | |
| Average | | | |

*ND = Not Done.

A preliminary study similar to that immediately above was carried out using Compounds 4, 9 and 10 and 100 nM of frozen-stored FITC-NLX rather than 10 nM FITC-NLX. The results of an average of two runs for this study are shown below.

| Compound | 0.1 nM | 1 nM | 10 nM | 100 nM | 1 mM |
|---|---|---|---|---|---|
| 4 | 18.8% | 21.3% | 17.9% | 28.8% | 42.9% |
| 9 | 22.5% | 24.8% | 27.7% | 35.3 | 49.6% |
| 10 | 27.5% | 27.3% | 26.6% | 27.3% | 34.5% |
| (+) NLX | 22.7% | 22.8% | 23.1% | 22.8% | 39.8% |

### EXAMPLE 2

### MOR Agonist Activity Using GTPgS Binding Assay

To assess the mu opiate receptor (MOR) agonist activity of positive compounds from the FLNA screening, compounds were tested in a [³⁵S]GTPgS binding assay using striatal membranes. A previous study has shown that in striatal membranes, activation of MOR leads to an increase in [³⁵S]GTPgS binding to Gao (Wang et al., 2005 Neuroscience 135:247-261). This assay measures a functional consequence of receptor occupancy at one of the earliest receptor-mediated events. The assay permits for traditional pharmacological parameters of potency, efficacy and antagonist affinity, with the advantage that agonist measures are not subjected to amplification or other modulation that may occur when analyzing parameters further downstream of the receptor.

Thus, striatal tissue was homogenized in 10 volumes of ice cold 25 mM HEPES buffer, pH 7.4, which contained 1 mM EGTA, 100 mM sucrose, 50 mg/ml leupeptin, 0.04 mM PMSF, 2 mg/ml soybean trypsin inhibitor and 0.2% 2-mercaptoethanol. The homogenates were centrifuged at 800×g for 5 minutes and the supernatants were centrifuged at 49,000×g for 20 minutes. The resulting pellets were suspended in 10 volume of reaction buffer, which contained 25 mM HEPES, pH 7.5, 100 mM NaCl, 50 mg/ml leupeptin, 2 mg/ml soybean trypsin inhibitor, 0.04 mM PMSF and 0.02% 2-mercaptomethanol.

The resultant striatal membrane preparation (200 mg) was admixed and maintained (incubated) at 30° C. for 5 minutes in reaction buffer as above that additionally contained 1 mM MgCl₂ and 0.5 nM [³⁵S]GTPgS (0.1 mCi/assay, PerkinElmer Life and Analytical Sciences) in a total volume of 250 ml and continued for 5 minutes in the absence or presence of 0.1-10 mM of an assayed compound of interest. The reaction was terminated by dilution with 750 ml of ice-cold reaction buffer that contained 20 mM MgCl₂ and 1 mM EGTA and immediate centrifugation at 16,000×g for 5 minutes.

The resulting pellet was solubilized by sonicating for 10 seconds in 0.5 ml of immunoprecipitation buffer containing 0.5% digitonin, 0.2% sodium cholate and 0.5% NP-40. Normal rabbit serum (1 ml) was added to 1 ml of lysate and incubated at 25° C. for 30 minutes. Nonspecific immune complexes were removed by incubation with 25 ml of protein A/G-conjugated agarose beads at 25° C. for 30 minutes followed by centrifugation at 5,000×g at 4° C. for 5 minutes. The supernatant was divided and separately incubated at 25° C. for 30 minutes with antibodies raised against Gao proteins (1:1,000 dilutions).

The immunocomplexes so formed were collected by incubation at 25° C. for 30 minutes with 40 ml of agarose-conjugated protein A/G beads and centrifugation at 5,000×g at 4° C. for 5 minutes. The pellet was washed and suspended in buffer containing 50 mM Tris-HCl, pH 8.0, and 1% NP-40. The radioactivity in the suspension was determined by liquid scintillation spectrometry. The specificity of MOR activation of [³⁵S]GTPgS binding to Gao induced by a selective compound was defined by inclusion of 1 mM b-funaltrexamine (b-FNA; an alkylating derivative of naltrexone that is a selective MOR antagonist). DAMGO (1 or 10 mM) was used as a positive control.

The results of this study are shown in the Tables below.

Series C-1 FLNA-Binding Compound MOR Agonist Activity

| | Concentration of FLNA-Binding Compound as Agonist | | | | | |
|---|---|---|---|---|---|---|
| FLNA-Binding | | | 1 μM + | % DAMGO | % DAMGO | % DAMGO + |
| Compound | 0.1 μM | 1 μM | BFNA | (0.1 μM) | (1 μM) | BFNA |
| 7866 | 152.3% | 308.2% | 62.4% | 79.3% | 94.8% | 129.5% |
| C0001 | 129.3% | 184.3% | 33.9% | 75.2% | 66.6% | 52.9% |
| C0002 | 88.4% | 93.8% | 3.9% | 51.4% | 33.9% | 6.1% |
| C0003 | 162.3% | 215.9% | 107.7% | 91.9% | 83.3% | 163.9% |
| C0004 | 122.0% | 228.4% | 65.8% | 72.1% | 85.4% | 99.7% |
| C0005 | 180.4% | 227.2% | 166.4% | 105.4% | 85.1% | 319.4% |
| C0006 | 121.5% | 204.0% | 4.6% | 70.6% | 73.8% | 7.2% |
| C0007 | 79.1% | 195.0% | 10.9% | 46.0% | 70.5% | 17.0% |
| C0008 | 71.2% | 201.6% | 2.8% | 41.4% | 72.9% | 4.4% |
| C0009 | 146.3% | 256.2% | 26.4% | 85.1% | 92.6% | 41.2% |
| C0010 | 136.5% | 307.0% | 89.1% | 80.7% | 114.9% | 135.0% |
| C0011 | 217.0% | 305.0% | 19.0% | 126.8% | 114.3% | 36.5% |
| C0012 | 96.8% | 224.8% | 184.4% | 54.8% | 86.7% | 280.7% |
| C0013 | 156.6% | 301.2% | 39.6% | 91.0% | 108.9% | 61.8% |
| C0014 | 144.9% | 153.5% | 76.3% | 82.0% | 59.2% | 116.1% |
| C0015 | 138.7% | 204.7% | 126.8% | 78.5% | 78.9% | 193.0% |
| C0016 | 172.7% | 230.5% | 96.7% | 100.4% | 83.3% | 150.9% |
| C0017 | 153.8% | 284.5% | 94.1% | 87.1% | 109.7% | 143.2% |
| C0018 | 195.5% | 247.7% | 106.5% | 110.7% | 95.5% | 162.1% |
| C0019 | 104.4% | 176.6% | 52.8% | 59.1% | 68.1% | 80.4% |
| C0021 | 159.7% | 192.0% | 90.7% | 94.5% | 87.8% | 546.4% |
| C0022 | 194.3% | 328.7% | 13.4% | 113.5% | 123.2% | 25.7% |
| C0023 | 153.2% | 233.7% | 23.2% | 89.5% | 87.6% | 44.5% |
| C0024 | 178.4% | 229.6% | 59.3% | 92.8% | 84.1% | 135.1% |
| C0025 | 235.7% | 320.7% | 80.2% | 122.6% | 117.5% | 182.7% |
| C0028 | 93.9% | 132.4% | 78.4% | 55.6% | 60.5% | 472.3% |
| C0029 | 175.4% | 308.8% | 16.6% | 91.2% | 113.1% | 37.8% |
| C0030 | 150.3% | 226.8% | 95.0% | 96.0% | 98.0% | 291.4% |
| C0032 | 145.4% | 202.0% | 80.9% | 92.8% | 87.3% | 248.2% |
| C0033 | 134.5% | 186.4% | 76.6% | 85.9% | 80.6% | 235.0% |
| C0034 | 103.6% | 167.9% | 80.1% | 61.3% | 76.7% | 482.5% |
| C0041 | 186.1% | 244.4% | 95.5% | 110.1% | 111.7% | 575.3% |
| C0042 | 167.1% | 260.9% | 110.6% | 98.9% | 119.2% | 666.3% |
| C0047 | 142.2% | 206.1% | 80.1% | 98.1% | 88.5% | 182.0% |
| C0048 | 209.1% | 245.3% | 89.9% | 144.2% | 105.3% | 204.3% |
| C0049 | 106.6% | 210.0% | 81.0% | 73.5% | 90.1% | 184.1% |
| C0051 | 94.4% | 170.4% | 55.9% | 65.1% | 73.1% | 127.0% |
| C0052 | 108.4% | 162.8% | 42.7% | 74.8% | 69.9% | 97.0% |
| C0053 | 104.0% | 157.2% | 93.1% | 71.7% | 67.5% | 211.6% |
| C0054 | 68.2% | 127.0% | 43.5% | 47.0% | 54.5% | 98.9% |
| C0057 | ND* | ND | ND | ND | ND | ND |
| C0061 | ND | ND | ND | ND | ND | ND |
| C0062 | 127.8% | 310.5% | 59.8% | 81.9% | 134.7% | 149.9% |
| C0064 | 213.8% | 349.6% | 38.1% | 124.2% | 159.1% | 110.4% |
| C0065 | 198.3% | 279.5% | 47.7% | 127.0% | 121.3% | 119.5% |
| C0067 | 142.7% | 179.0% | 33.5% | 82.9% | 81.5% | 97.1% |
| C0068 | 107.2% | 263.1% | 165.9% | 53.4% | 83.8% | 307.8% |
| C0069 | ND | ND | ND | ND | ND | ND |
| C0070 | 165.6% | 210.8% | 114.2% | 96.2% | 95.9% | 331.0% |
| C0071 | 276.3% | 355.3% | 177.1% | 160.5% | 161.7% | 513.3% |
| C0072 | 172.7% | 259.1% | 67.1% | 100.3% | 117.9% | 194.5% |
| C0073 | ND | ND | ND | ND | ND | ND |
| C0077 | 192.7% | 265.4% | 136.7% | 109.5% | 104.9% | 621.4% |
| C0078 | 138.1% | 236.6% | 170.7% | 82.4% | 106.4% | 359.4% |
| C0080M | 187.9% | 205.4% | 167.1% | 112.1% | 92.4% | 351.8% |
| C0082M | 228.1% | 338.4% | 97.6% | 113.7% | 107.8% | 181.1% |
| C0084M | 163.1% | 255.5% | 133.2% | 97.3% | 114.9% | 280.4% |
| C0085M | 211.6% | 246.2% | 43.7% | 105.5% | 78.4% | 112.6% |
| C0138M | 126.9% | 183.9% | 51.5% | 86.3% | 90.9% | 131.0% |
| C0139M | 156.1% | 206.6% | 51.0% | 106.2% | 102.2% | 129.8% |
| C0140M | 126.1% | 215.4% | 83.0% | 85.8% | 106.5% | 211.2% |
| C0141M | 161.5% | 213.9% | 47.9% | 109.9% | 105.8% | 121.9% |
| C0143M | 81.0 | 193.3 | 86.5 | 47.1% | 59.3% | 94.7% |
| C0144M | 186.3 | 295.9 | 125.9 | 108.3% | 90.8% | 137.9% |
| C0145M | 193.0 | 289.2 | 87.0 | 112.2% | 88.7% | 95.3% |
| C0146M | ND | ND | ND | ND | ND | ND |
| C0147M A2 | ND | ND | ND | ND | ND | ND |
| C0148M A2 | 181.3 | 360.6 | 87.6 | 105.4% | 110.6% | 95.9% |
| C0149M | 209.8 | 406.7 | 93.4 | 122.0% | 124.8% | 102.3% |
| C0150M | 167.1 | 423.1 | 93.4 | 9 7.2% | 129.8% | 173.2% |
| C0151M | 346.8 | 397.6 | 212.8 | 201.6% | 122.0% | 233.1% |
| C0152M | ND | ND | ND | ND | ND | ND |
| DAMGO | 168.5% | 266.1% | 53.2% | ND | ND | ND |
| Average | | | | | | |

*ND = Not Done.

Series C-2 FLNA-Binding Compound MOR Agonist Activity

| FLNA- | Concentration of FLNA-Binding Compound as Agonist | | | | | |
|---|---|---|---|---|---|---|
| Binding | | | 1 μM + | % DAMGO | % DAMGO | % DAMGO + |
| Compound | 0.1 μM | 1 μM | BFNA | (0.1 μM) | (1 μM) | BFNA |
| C0011 | 217.0% | 305.0% | 19.0% | 126.8% | 114.3% | 36.5% |
| C0026 | 207.2% | 288.4% | 21.2% | 107.7% | 105.6% | 48.3% |
| C0027 | 233.2% | 313.9% | 72.2% | 121.3% | 115.0% | 164.5% |
| S-C0027 | 156.2% | 286.8% | 56.2% | 74.2% | 84.4% | 98.1% |
| C0034-3 | ND* | ND | ND | ND | ND | ND |
| C0037-2 | ND | ND | ND | ND | ND | ND |
| C0040 | 145.8% | 308.3% | 90.4% | 93.1% | 133.2% | 277.3% |
| C0043 | 175.4% | 242.6% | 83.3% | 103.8% | 110.9% | 501.8% |
| C0044 | 173.7% | 280.1% | 59.1% | 102.8% | 128.0% | 356.0% |
| C0045 | 149.2% | 238.8% | 105.3% | 88.3% | 109.1% | 634.3% |
| C0046 | 286.2% | 492.9% | 156.8% | 197.4% | 211.5% | 356.4% |
| C0050 | 110.3% | 127.6% | 59.0% | 76.1% | 54.8% | 134.1% |
| C0055 | ND | ND | ND | ND | ND | ND |
| C0056 | 98.6% | 193.4% | 86.3% | 68.0% | 83.0% | 196.1% |
| C0060 | 166.5% | 218.9% | 143.9% | 114.8% | 93.9% | 327.0% |
| C0086M | 206.8% | 265.3% | 152.3% | 117.5% | 104.9% | 692.3% |
| C0087M | 262.8% | 329.6% | 142.5% | 138.9% | 132.8% | 293.8% |
| C0088M | 276.3% | 355.3% | 177.1% | 160.5% | 161.7% | 513.3% |
| C0089M | 234.5% | 295.3% | 81.9% | 136.3% | 134.4% | 237.4% |
| C0090M | 237.0% | 341.0% | 41.0% | 137.7% | 155.2% | 118.8% |
| C0091M | 207.9% | 274.4% | 80.8% | 118.1% | 108.5% | 367.3% |
| C0093M | 140.0% | 211.8% | 44.0% | 81.3% | 96.4% | 127.5% |
| C0094M | 172.5% | 263.5% | 115.3% | 100.2% | 119.9% | 334.2% |
| C0095M | 189.1% | 224.6% | 107.7% | 107.4% | 88.8% | 489.5% |
| C0096M | 186.4% | 328.9% | 127.1% | 105.9% | 130.0% | 577.7% |
| C0099M | 157.2% | 195.7% | 114.7% | 93.8% | 88.0% | 241.5% |
| C0100M | 173.6% | 245.9% | 195.6% | 103.6% | 110.6% | 411.8% |
| C0101M | 138.2% | 274.3% | 174.8% | 82.5% | 123.4% | 368.0% |
| C0102M | 131.8% | 272.0% | 150.4% | 78.6% | 122.4% | 316.6% |
| C0104M | 188.2% | 238.9% | 143.8% | 99.5% | 96.3% | 296.5% |
| C0105M | 198.1% | 220.3% | 73.1% | 104.7% | 88.8% | 150.7% |
| C0106M | 171.8% | 240.7% | 117.2% | 102.5% | 108.3% | 246.7% |
| C0108M | 205.6% | 258.5% | 76.9% | 108.7% | 104.1% | 158.6% |
| C0114M | 114.0% | 144.3% | 35.9% | 77.6% | 71.4% | 91.3% |
| C0115M | 177.2% | 226.8% | 118.4% | 105.7% | 102.0% | 249.3% |
| C0116M | 258.4% | 302.8% | 152.0% | 136.6% | 122.0% | 313.4% |
| C0118M | 166.2% | 261.5% | 79.2% | 87.8% | 105.4% | 163.3% |
| C0119M | 105.7% | 167.8% | 35.1% | 71.9% | 83.0% | 89.3% |
| C0124M | 252.0% | 305.1% | 61.4% | 133.2% | 122.9% | 126.6% |
| C0125M | 168.6% | 195.2% | 159.7% | 89.1% | 78.6% | 329.3% |
| C0126M | 181.8% | 265.3% | 108.5% | 108.5% | 119.3% | 228.4% |
| C0128M | 197.8% | 286.0% | 63.9% | 104.5% | 115.2% | 131.8% |
| C0133M | 139.4% | 214.8% | 72.4% | 83.2% | 96.6% | 152.4% |
| C0134M | 158.5% | 207.3% | 46.6% | 94.6% | 93.3% | 98.1% |
| F-C0134 | 290.6% | 378.9% | 66.6% | 138.1% | 111.4% | 116.2% |
| C0135M | 161.3% | 310.1% | 113.3% | 85.3% | 124.9% | 233.6% |
| C0136M | 176.8% | 237.3% | 74.5% | 93.4% | 95.6% | 153.6% |
| (P5) | | | | | | |
| C0137M | 180.8% | 193.8% | 55.8% | 95.6% | 78.1% | 115.1% |
| (P7) | | | | | | |
| C0142M | 143.7% | 192.5% | 98.7% | 97.8% | 95.2% | 251.1% |
| C0143M | 81.0% | 193.3% | 86.5% | 47.1% | 59.3% | 94.7 |
| C0144M-2 | 186.3% | 295.9% | 125.9% | 108.3% | 90.8% | 137.9% |
| C0145M-3 | 193.0% | 289.2% | 87.0% | 112.2% | 88.7% | 95.3% |
| C0149M-2 | 209.8% | 406.7% | 93.4% | 122.0% | 124.8% | 102.3% |
| C0150M-2 | 167.1% | 423.1% | 158.1% | 97.2% | 129.8% | 173.2% |
| C0151M-2 | 346.8% | 397.6% | 212.8% | 201.6% | 122.0% | 233.1% |
| C0152M-2 | ND | ND | ND | ND | ND | ND |
| C0153M-3 | ND | ND | ND | ND | ND | ND |
| DAMGO | 168.5% | 266.1% | 53.2% | ND | ND | ND |
| Average | | | | | | |

*ND = Not Done.

A preliminary study similar to that immediately above was carried out using Compounds 4, 9 and 10 and resynthesized Compound C0134M and DAMGO. The results of an average of two runs for this study are shown below.

| | Concentration of FLNA-Binding | | |
|---|---|---|---|
| | Compound as Agonist | | |
| Compound | 0.1 mM | 1 mM | 1 mM + bNFA |
| 4 | 133.9% | 165.2% | 49.5% |
| 9 | 156.6% | 197.2% | 56.6% |
| 10 | 163.1% | 191.8% | 60.4% |
| C0134M | 150.7% | 224.0% | 53.2% |
| DAMGO | 144.7% | 233.4% | 56.8% |

The above results indicate that Compounds 9 and 10 not only bind well to FLNA, but are also MOR agonists, whereas Compound 4 bound well to FLNA, but was not as potent a MOR agonist as were the other two compounds. The newly synthesized Compound C0134M exhibited similar MOR agonist activity to that shown previously.

### Materials and Methods

An in vitro study was conducted under the direction of Hoau-Yan Wang, Ph.D. by the Dept. of Physiology, Pharmacology & Neuroscience, CUNY Medical School, 138th Street and Convent Avenue, New York, N.Y. 10031, to assess the top two filamin A (FLNA)-binding compounds, C0105 and C0114 for the ability to block amyloid beta₄₂ (Ab₄₂)-induced FLNA-a7 nicotinic acetylcholine receptor (a7nAChR) association and tau phosphorylation, indicating the potential to treat Alzheimer's disease.

### Animals

Adult Sprague Dawley rats (2 months old) were used for organotypic frontocortical slice cultures. Rats were maintained on a 12-hour light/dark cycle with food and water. All animal procedures comply with the National Institutes of Health Guide for Care Use of Laboratory Animals and were approved by the City College of New York Animal Care and Use Committee.

### Organotypic Frontocortical Slice Cultures

Rat brain slice organotypic culture methods were modified from those published previously. [Adamchik et al., Brain Res Brain Res Protoc 5:153-158 (2000).] FCX slices (200 mM thickness) were transferred to sterile, porous Millicell-CM inserts (0.4 mm). Each culture insert unit contained two brain slices and was placed into individual wells of a 12-well culture tray in 2 ml medium: 50% MEM with Earl's salts, 2 mM L-glutamine, 25% Earl's balanced salt solution, 6.5 g/l D-glucose, 20% fetal bovine serum (FBS), 5% horse serum, 25 mM HEPES buffer, pH 7.2, and 50 mg/ml streptomycin and 50 mg/ml penicillin. Cultures were kept in an incubator for 2 days at 36° C. in 5% CO₂ to minimize the impact of injury from slice preparation.

On the day of experiment, medium was removed, the brain slices rinsed and incubated in 0.1% FBS-containing medium for 4 hours at 36° C. in 5% CO₂. Brain slices were then cultured with 100 nM Ab₄₂ and/or 0.1, 1 nM compound C0105 or compound C0114 in fresh 0.1% FBS-containing medium for 16 hours. Brain slices (6 slices for each experiment) were washed with ice-cold Krebs-Ringer and used to assess a7nAChR-FLNA complex level and phosphorylated tau (pS²⁰²-, pT²³¹- and pT¹⁸¹-tau). Brain slices were also used to determine the a7nAChR and NMDAR activity by the level of calcium influx through each of these two channels and the level of cell death using voltage-gated calcium channel mediated calcium influx.

For immunohistochemistry, additional slices were removed and fixed in 4% paraformaldehyde in PBS at 4° C. The effect of C0105 and C0114 on intraneuronal Ab₄₂ accumulation was determined by the Ab₄₂ immunostaining level.

### Brain Synaptosome Preparation

Brain synaptosomes (P2 fraction) were prepared from FCX slice cultures. Following methods described previously, [Wang et al., J Biol Chem 278:31547-31553 (2003)] FCX was solubilized immediately after removal from cultures to obtain synaptosomes. The synaptosomes were washed twice and suspended in 2 ml of ice-cold Krebs-Ringer (K-R): 25 mM HEPES, pH 7.4; 118 mM NaCl, 4.8 mM KCl, 25 mM NaHCO₃, 1.3 mM CaCl₂, 1.2 mM MgSO₄, 1.2 mM KH₂PO₄, 10 mM glucose, 100 mM ascorbic acid, mixture of protease and protein phosphatase inhibitors (Roche Diagnostics) that had been aerated for 10 minutes with 95% O₂/5% CO₂. The protein concentration was determined using the Bradford method (Bio-Rad).

### In Vitro Studies Using Organotypic FCX Tissues

To assess the effect of compounds C0105 and C0114 on Ab₄₂-induced a7nAChR-FLNA interaction and tau phosphorylation (pS²⁰²-, pT²³¹- and pT¹⁸¹-tau) levels, rat frontal cortical slice culture system was used. Rat brain FCX were chopped coronally into 200 mm slices using a Mcllwain chopper (Brinkman Instruments) and suspended in 10 ml of ice-cold oxygenated K-R.

The rat brain slice organotypic culture was performed as described previously. [Wang et al., Biol Psychiatry 67, 522-530 (2010).] Rat FCX slices were transferred to sterile, porous 0.4 mm Millicell-CM insert, 2 slices per insert per well containing 2 ml medium: 50% MEM with Earl's salts, 2 mM L-glutamine, 25% Earl's balanced salt solution, 6.5 g/l D-glucose, 20% fetal bovine serum (FBS), 5% horse serum, 25 mM HEPES buffer, pH 7.2, and 50 mg/ml streptomycin and 50 mg/ml penicillin. Cultures were kept in an incubator for 2 days at 36° C. in 5% CO₂.

On the day of study, medium was removed, the brain slices rinsed and incubated in 0.1% FBS-containing medium for 4 hours at 36° C. in 5% CO₂. Brain slices were then cultured with 0.1 mM Ab₄₂ together with 0.1, 1 or 10 nM Compound C0105 or 1 or 10 nM C0114 in fresh 0.1% FBS-containing medium for 16 hours. The brain slices were then removed and washed with ice-cold PBS three times and either processed for functional assays described below or fixed in ice-cold 4% paraformaldehyde PBS at 4° C. for determination of intraneuronal Ab₄₂ aggregate and NFT levels by immunohistochemical method.

1) FLNA Association with a7nAChR and Other Receptors

The level of FLNA-associated a7nAChRs was determined using a co-immunoprecipitation/Western blotting method as described previously. [Wang et al., Biol Psychiatry 67:522-530 (2010); Wang et al., PLoS One 3:e1554 (2008); Wang et al., J Neurosci 35:10961-10973 (2009)] Briefly, brain slice extract (200 mg) was incubated with 1 mg anti-FLNA immobilized on protein A agarose beads at 4° C. overnight (about 18 hours) with constant end-over-end rotation. The anti-FLNA immunocomplexes were obtained by centrifugation, and then washed and dissociated using antigen elution buffer. Following neutralization with 1.5M Tris, pH 8.8, the resultant FLNA-associated protein complexes were solubilized by boiling for 5 minutes in SDS-containing sample preparation buffer. The levels of FLNA-associated a7nAChR, TLR2, IR and MOR were assessed by Western blotting using specific antibodies directed against the respective proteins and the blot stripped and re-probed for FLNA for immunoprecipitation/loading control.

2) Tau Phosphorylation

Using an established method, [Wang et al., J Biol Chem 278:31547-31553 (2003); Wang et al., Biol Psychiatry 67:522-530 (2010)] tau proteins were immunoprecipitated with immobilized anti-tau (SC-65865), which does not discriminate between phosphorylation states. The levels of phosphorylated tau (pSer202tau, pThr231tau and pThr181tau) as well as total tau precipitated (loading controls) are assessed by Western blotting using specific antibodies directed against each of the phosphor-epitopes and the anti-tau, respectively.

3) Functional Assessment of a7nAChR and NMDAR

The effect of Compounds C0105 and C0114 on a7nAChR and NMDAR function was assessed in organotypic FCX slice cultures treated with vehicle, 0.1 mM Ab₄₂ or 0.1 mM Ab₄₂+0.1-10 nM C0105 or 1-10 nM C0114. Synaptosomes prepared from rat FCX slices (6 slices/assay) were washed twice in ice-cold K-R, centrifuged and re-suspended in 0.5 ml K-R.

NMDAR and a7nAChR mediated ⁴⁵Ca²⁺ influx was measured as described previously. [Wang et al., Biol Psychiatry 67:522-530 (2010).] Synaptosomes (50 mg) were incubated at 37 C for 5 minutes in oxygenated 0.3 mM Mg²⁺ K-R containing 5 mM ⁴⁵Ca²⁺ (10 Ci/mmol, PerkinElmer) followed by incubation with vehicle, 0.1-10 mM NMDA/1 mM glycine or 0.1-10 mM PNU282987 (a potent and selective agonist for the α7 subtype of neural nicotinic acetylcholine receptors) for 5 minutes. The reaction was terminated by 1 ml ice-cold 0.5 mM EGTA-containing Ca²⁺-free K-R and centrifugation. After two washes, synaptosomal ⁴⁵Ca²⁺ contents were assessed using scintillation spectrometry.

The background ⁴⁵Ca²⁺ was estimated using hypotonically lysed synaptosomes. The absolute Ca²⁺ influx was calculated by subtracting background ⁴⁵Ca²⁺ count. The percent increase in Ca²⁺ influx was calculated as % [(drug-treated−vehicle)/vehicle].

4) Cell Death Measured by K⁺-Evoked Ca⁺² Influx

Because the level of voltage-gated Ca²⁺ channel activity is indicative of the integrity of the cells, the effect of compounds C0105 and C0114 on Ab₄₂-induced cell death was assessed in organotypic FCX slice cultures treated with vehicle, 0.1 mM Ab₄₂ or 0.1 mM Ab₄₂+0.1-10 nM compound C0105 or 1-10 nM compound C0114 using K⁺-depolarization mediated Ca²⁺ influx. Synaptosomes prepared from rat FCX slices (6 slices/assay) were washed twice in ice-cold K-R, centrifuged and re-suspended in 0.5 ml K-R. The level of voltage-gated Ca²⁺ channel mediated ⁴⁵Ca²⁺ influx was measured as described previously. [Wang et al., Biol Psychiatry 67:522-530 (2010).]

Synaptosomes (50 mg) were incubated at 37 C for 5 minutes in oxygenated 0.3 mM Mg²⁺ K-R containing 5 mM ⁴⁵Ca²⁺ (10 Ci/mmol, PerkinElmer) followed by incubation with vehicle or 65 mM K⁺ (made with isomolar replacement of Na⁺) for 1 minute. The reaction was terminated by 1 ml ice-cold 0.5 mM EGTA-containing Ca²⁺-free K-R and centrifugation. After two washes, synaptosomal ⁴⁵Ca²⁺ contents were assessed using scintillation spectrometry. The background ⁴⁵Ca²⁺ was estimated using hypotonically lysed synaptosomes. The absolute Ca²⁺ influx was calculated by subtracting background ⁴⁵Ca²⁺ count. The percent increase in Ca²⁺ influx was calculated as % [(drug-treated−vehicle)/vehicle].

5) Measuring Levels of Signaling Molecules Associated with NMDAR or IR After Receptor Stimulation

NMDAR signaling and their interaction with synaptic anchoring protein, PSD-95, were compared in brain slices from organotypic culture FCX treated with vehicle, 0.1 mM Ab₄₂ and 0.1 mM Ab₄₂+0.1-10 nM of compound C0105 or 1 and 10 nM of compound C0114 for 16 hours. NMDAR activation and signaling was initiated by incubation of 6 slices with either 0.3 mM Mg²⁺ containing KR (LMKR; basal) or LMKR containing 10 mM NMDA and 1 mM glycine at 37 C for 30 minutes.

The incubation mixture was aerated with 95% O₂/5% CO₂ every 10 minutes for 1 minute during the stimulation. Ligand stimulation was terminated by the addition of 1 ml of ice-cold Ca²⁺-free K-R containing mixture of protein phosphatase inhibitors, 0.5 mM EGTA and 0.1 mM EDTA. Brain slices were harvested by a brief centrifugation and were homogenized in 0.25 ml of ice-cold immunoprecipitation buffer. The homogenates were centrifuged at 1000·g for 5 minutes (4 C) and the supernatant (post-mitochondrial fraction) was sonicated for 10 seconds on ice.

The proteins were solubilized in 0.5% digitonin, 0.2% sodium cholate and 0.5% NP-40 for 60 minutes at 4 C with end-over-end rotation. The resultant lysates were cleared by centrifugation at 50,000·g for 5 minutes and diluted with 0.75 ml of immunoprecipitation buffer. Protein concentrations were measured by Bradford method (Bio-Rad).

To determine the association of NMDARs with PSD-95, as well as NMDAR signaling, the levels of NMDAR subunits, PSD-95 and NMDAR-associated signaling molecules were measured in anti-NR1 immunoprecipitates. In these studies, brain slice lysates (100 mg) were immunoprecipitated overnight at 4° C. with 2 mg of immobilized anti-NR1 onto covalently conjugated protein A-agarose beads (Pierce-ENDOGEN). Anti-NR1 immunoprecipitates were incubated with 75 ml antigen elution buffer (Pierce-ENDOGEN) and 2% SDS for 2 minutes on ice, centrifuged to remove antibody-protein A-agarose complexes and neutralized immediately with 10 ml 1.5 M Tris buffer, pH 8.8 followed by addition of 65 ml 2×PAGE sample buffer and boiled for 5 minutes.

Seventy-five ml of the obtained eluates (50%) were size fractionated on 7.5% SDS-PAGE. Proteins were transferred to nitrocellulose membrane and the levels of various NMDA receptor subunits, PSD-95, signaling proteins were measured using Western blotting with antibodies for PSD-95, nNOS, phospholipase C-g1, gPKC, pY⁴⁰²PyK2, pY⁴¹⁶Src or phosphotyrosine. The blots were stripped and re-probed with anti-NR1 to assess the immunoprecipitation efficiency and loading.

IR activation and signaling was initiated by incubation of 6 slices that were further chopped horizontally into 100 mm (100 mm×200 mm×3 mm) with either KR (basal) or KR containing 1 nM insulin at 37 C for 30 minutes. The incubation mixture was aerated with 95% O₂/5% CO₂ every 10 minutes for 1 minute during the stimulation. Ligand stimulation was terminated by the addition of 1 ml of ice-cold Ca²⁺-free K-R containing mixture of protein phosphatase inhibitors, 0.5 mM EGTA and 0.1 mM EDTA. Brain slices were harvested by a brief centrifugation and were homogenized in 0.25 ml of ice-cold immunoprecipitation buffer. The homogenates were centrifuged at 1000·g for 5 minutes (4 C) and the supernatant (post-mitochondrial fraction) was sonicated for 10 seconds on ice. The proteins were solubilized in 0.5% digitonin, 0.2% sodium cholate and 0.5% NP-40 for 60 minutes at 4 C with end-over-end rotation. The resultant lysates were then cleared by centrifugation at 50,000·g for 5 minutes and diluted with 0.75 ml of immunoprecipitation buffer. Protein concentrations were measured by Bradford method (Bio-Rad).

To determine the IR activation and signaling, the levels of pY^{1150/1151}IRb and the level of IR signal transducer, IRS-1 were measured in anti-IRb immunoprecipitates. In these experiments, brain slice lysates (100 mg) were immunoprecipitated overnight (about 18 hours) at 4° C. with 2 mg of immobilized anti-IRb onto covalently conjugated protein A-agarose beads (Pierce-ENDOGEN).

Anti-IRb immunoprecipitates were incubated with 75 ml antigen elution buffer (Pierce-ENDOGEN) and 2% SDS for 2 min on ice, centrifuged to remove antibody-protein A-agarose complexes and neutralized immediately with 10 ml 1.5 M Tris buffer, pH 8.8 followed by addition of 65 ml 2×PAGE sample buffer and boiled for 5 minutes. Seventy-five ml of the obtained eluates (50%) were then size fractionated on 7.5% SDS-PAGE. Proteins were transferred to nitrocellulose membrane and the levels of pY^{1150/1151}IRb and IRS-1 proteins were measured using Western blotting with antibodies for pY^{1150/1151}IRb and IRS-1. The blots were stripped and re-probed with anti-IRb to assess the immunoprecipitation efficiency and loading.

6) Immunohistochemical Studies

Quantitative immunohistochemistry on consecutive 5-mm sections containing PFCX and entorhinal cortex/HP were used to determine the levels of Ab₄₂ aggregates/plaques and neurofibrillary pathology (NFT and paired helical filament [PHF] immunoreactivity) using single labeling immunohistochemistry as described previously. [Wang et al., Biol Psychiatry 67:522-530 (2010); D'Andrea et al., Histopathology 38:120-134 (2001); Nagele et al., Neuroscience 110:199-211 (2002).] One section was immunostained with anti-NFT or -PHF. The next (consecutive) section (often containing the same neuron) was immunostained with anti-Ab₄₂ antibodies to measure relative levels of accumulated Ab₄₂ peptide in neurons. The relative Ab₄₂ accumulation rate/extent were compared among different cell types in sections from cultured FCX slices and icv Ab₄₂-infused mouse brains using a computer-assisted image analysis as described previously [Wang et al. J Biol Chem 275:5626-5632 (2000)].

Brain slices were fixed at 4° C. in 0.15 M phosphate-buffered 10% formalin, pH 7.4 for 2 weeks, paraffin embedded, serially sectioned at 5 mm, and processed for brightfield immunohistochemistry as described previously [Wang et al., J Biol Chem 275:5626-5632 (2000)]. The Ab₄₂ immunoreactivity was absent when pre-absorbing anti-Ab₄₂ with Ab₄₂ but not Ab₄₂₋₁. Specimens were examined using a Nikon FXA microscope with a Princeton Instruments CCD camera and recorded digitally.

Relative intensities of the NFT/PHF and Ab₄₂ immunoreactivity were measured and compared among similar and different cell types using Image Pro Plus and Metamorph software as described previously [D'Andrea et al., Histopathology 38:120-134 (2001)]. The correlations between the amount of NFT/PHF immunoreactivity and Ab₄₂-positive material accumulated within mature neurons were also determined.

### In Vivo Studies

An in vivo study was conducted under the direction of Hoau-Yan Wang, Ph.D. by the Dept. of Physiology, Pharmacology & Neuroscience, CUNY Medical School, 138th Street and Convent Avenue, New York, N.Y. 10031, in an amyloid beta₄₂ (Ab₄₂) infusion model of Alzheimer's disease for the ability 1) to block Ab₄₂-induced FLNA association with a7 nicotinic acetylcholine receptor (a7nAChR) and toll-like receptor 4 (and/or TLR2), 2) tau phosphorylation, and 3) Ab₄₂-a7nAChR association indicating the potential to treat Alzheimer's disease.

### ICV Ab₄₂ Infusion Mouse Model

Mice

Eight-week-old male and female E129 mice (30-35 g), progeny of the breeding pairs from Taconic (Germantown, N.Y.) were used in the intracerebroventricular (ICV) Ab₄₂ study. Mice were maintained on a 12-hour light/dark cycle with food and water. All animal procedures comply with the National Institutes of Health Guide for Care Use of Laboratory Animals and were approved by the City College of New York Animal Care and Use Committee.

### Intracerebroventricular Ab₄₂ Administration and Compound Treatment

Mice anesthetized with 30 mg/kg sodium pentobarbital intraperitoneally were placed in a mouse stereotaxic surgery apparatus as described by Wang et al., Biol Psychiatry 67:522-530 (2010). Mice receiving 7-day continuous ICV Ab₄₂ infusion were implanted with a minipump for mice (Alzet) that delivers 0.1 ml/hr through a surgical glue-secured cannula placed in the left ventricle at the following coordinates: [anterior-posterior from bregma, 3.0 mm; lateral, 1.0 mm; horizontal, 3.0 mm]. The Ab₄₂ (0.2 nmol/ml) was dissolved in 10% DMSO containing 50 mM Tris, pH 9.0, to prevent aggregation. Each mouse received 4.8 nmol Ab₄₂ daily for 7 days. Control mice received 7-day ICV infusion of vehicle.

To assess the effect of in vivo Compound C0105 on Ab₄₂-elicited effects, mice received 10 mg/kg of Compound C0105 by intraperitoneal (i.p.) injection daily for 2 weeks starting on the day of surgery (day 1: 2 hours after recovery from surgery, day 2-14 twice daily: between 10-11 a.m. and 3-4 p.m.). Twenty-four hours after the last injection, FCX and hippocampus from one half brain was solubilized for assessment of a7nAChR-FLNA complex level and phosphorylated tau (pS²⁰²-, pT²³¹- and pT¹⁸¹-tau) using published methods [Wang et al., Biol Psychiatry 67:522-530 (2010)].

Whether the compounds have an effect on levels of Ab₄₂-a7nAChR coupling was assessed because dissociating Ab₄₂ from a7nAChRs is beneficial in reducing AD pathologies. [Wang et al., Biol Psychiatry 67:522-530 (2010); Wang et al., J Neurosci 35:10961-10973 (2009).] In addition, prefrontal cortex (PFCX) is used to determine the level of synaptic activity using a7nAChR and NMDAR activity as the guide. The other brain halves were immersion-fixed in cold 0.15 M phosphate-buffered 10% formalin, pH 7.4, and processed for immunohistochemical determinations of intraneuronal Ab₄₂ aggregates/plaques and NFTs as well as morphological integrity.

### Brain Synaptosome Preparation

Brain synaptosomes (P2 fraction) were prepared from prefrontal cortex and hippocampus of treated mice sacrificed by rapid decapitation. Following methods described previously [Wang et al., J Biol Chem 278:31547-31553 (2003)], tissue was solubilized immediately after harvesting to obtain synaptosomes. The synaptosomes were washed twice and suspended in 2 ml of ice-cold Krebs-Ringer (K-R): 25 mM HEPES, pH 7.4; 118 mM NaCl, 4.8 mM KCl, 25 mM NaHCO₃, 1.3 mM CaCl₂, 1.2 mM MgSO₄, 1.2 mM KH₂PO₄, 10 mM glucose, 100 mM ascorbic acid, mixture of protease and protein phosphatase inhibitors (Roche Diagnostics) that had been aerated for 10 minutes with 95% O₂/5% CO₂. The protein concentration was determined using the Bradford method (Bio-Rad).

Ex Vivo Assessments of Tissues from Treated Mice

Using synaptosomes prepared from prefrontal cortex or hippocampi of mice receiving continuous ICV infusions of vehicle or Ab₄₂ and twice daily i.p. injections of Compound C0105 or vehicle, these studies assessed the effect of Compound C0105 on Ab₄₂-induced a7nAChR-FLNA interaction, tau phosphorylation (pS²⁰²-, pT²³¹- and pT¹⁸¹-tau) levels, Ab₄₂-a7nAChR interaction and signaling impairments.

1) a7nAChR-FLNA/TLR2 Interaction

The level of FLNA-associated a7nAChRs and TLR2s were determined using a co-immunoprecipitation/Western blotting method as described previously [Wang et al., Biol Psychiatry 67:522-530 (2010); Wang et al., J Neurosci 35:10961-10973 (2009); and Wang et al., PLoS One 3:e1554 (2008)]. Briefly, synaptosomal extracts (200 mg) prepared from prefrontal cortex or hippocampus from treated mice were incubated with 1 mg anti-FLNA immobilized on protein A agarose beads at 4° C. overnight (about 18 hours) with constant end-over-end rotation. The anti-FLNA immunocomplexes were obtained by centrifugation, washed and dissociated using antigen elution buffer. Following neutralization with 1.5M Tris, pH 8.8, the resultant FLNA-associated protein complexes were solubilized by boiling for 5 minutes in SDS-containing sample preparation buffer. The levels of FLNA-associated a7nAChRs and TLR2s were assessed by Western blotting and the blot stripped and re-probed for FLNA for immunoprecipitation/loading control.

To assess the effect of elevated Ab₄₂ and Compound C0105 treatment on FLNA and a7nAChR expression, FLNA and a7nAChR levels were measured in the tissue extract by Western blotting with b-actin as the loading control.

2) Tau Phosphorylation

Using established methods [Wang et al., Biol Psychiatry 67:522-530 (2010); and Wang et al., J Biol Chem 278:31547-31553 (2003)], tau proteins were immunoprecipitated with immobilized anti-tau (SC-65865), which does not discriminate between phosphorylation states. The levels of phosphorylated tau (pSer202tau, pThr231tau and pThr181tau) as well as total tau precipitated (loading controls) were assessed by Western blotting using specific antibodies directed against each of the phosphoepitopes and the anti-tau, respectively.

3) Ab₄₂-a7nAChR Interaction

The level of Ab₄₂-a7nAChR complexes were measured in synaptosomes from prefrontal cortex and hippocampus of treated mice using an established method [Wang et al., Biol Psychiatry 67:522-530 (2010); and Wang et al., J Biol Chem 278:31547-31553 (2003)]. Briefly, Ab₄₂-a7nAChR complexes were immunoprecipitated with immobilized anti-Ab₄₂ and the a7nAChR contents were measured by Western blotting. Anti-actin was added to immunoprecipitation and the b-actin level in the immunoprecipitates served as immunoprecipitation/loading control.

4) Functional Assessment of a7nAChR and NMDAR

The effect of Compound C0105 on a7nAChR and NMDAR function was assessed in mice infused with Ab₄₂ or vehicle. Synaptosomes prepared from prefrontal cortex or hippocampus were washed twice in ice-cold K-R, centrifuged and re-suspended in 0.5 ml K-R.

NMDAR and a7nAChR mediated ⁴⁵Ca²⁺ influx were measured as described previously [Wang et al., Biol Psychiatry 67:522-530 (2010)]. Synaptosomes (50 mg) were incubated at 37 C for 5 minutes in oxygenated 0.3 mM Mg²⁺ K-R containing 5 mM ⁴⁵Ca²⁺ (10 Ci/mmol, PerkinElmer) followed by incubation with vehicle, 0.1-10 mM NMDA/1 mM glycine or 0.1-10 mM PNU282987 for 5 minutes. The reaction was terminated by admixture of 1 ml ice-cold 0.5 mM EGTA-containing Ca²⁺-free K-R and centrifugation.

After two washes, synaptosomal ⁴⁵Ca²⁺ contents were assessed using scintillation spectrometry. The background ⁴⁵Ca²⁺ was estimated using hypotonically lysed synaptosomes. The absolute Ca²⁺ influx was calculated by subtracting background ⁴⁵Ca²⁺ count. The percent increase in Ca²⁺ influx was calculated as % [(drug-treated−vehicle)/vehicle].

5) Cell Death Measured by K+-Evoked Ca+2 Influx

Because the level of voltage-gated Ca²⁺ channel activity is indicative of the integrity of the cells, the effect of Compound C0105 on Ab₄₂-induced cell death was assessed in treated mice using K⁺-depolarization mediated Ca²⁺ influx. Synaptosomes prepared from prefrontal cortex were washed twice in ice-cold K-R, centrifuged and re-suspended in 0.5 ml K-R.

The level of voltage-gated Ca²⁺ channel mediated ⁴⁵Ca²⁺ influx was measured as described previously [Wang et al., Biol Psychiatry 67:522-530 (2010)]. Synaptosomes (50 mg) were incubated at 37 C for 5 minutes in oxygenated 0.3 mM Mg²⁺ K-R containing 5 mM ⁴⁵Ca²⁺ (10 Ci/mmol, PerkinElmer) followed by incubation with vehicle or 65 mM K⁺ (made with isomolar replacement of Na⁺) for 1 minute. The reaction was terminated by admixture of 1 ml ice-cold 0.5 mM EGTA-containing Ca²⁺-free K-R and centrifugation.

After two washes, synaptosomal ⁴⁵Ca²⁺ content was assessed using scintillation spectrometry. The background ⁴⁵Ca²⁺ was estimated using hypotonically lysed synaptosomes. The absolute Ca²⁺ influx was calculated by subtracting background ⁴⁵Ca²⁺ count. The percent increase in Ca²⁺ influx was calculated as % [(drug-treated−vehicle)/vehicle].

6) Measuring Levels of Signaling Molecules Associated with NMDAR or IR After Receptor Stimulation

NMDAR signaling and their interaction with synaptic anchoring protein, PSD-95 were compared in synaptosomes from treated mice. NMDAR activation and signaling was initiated by incubation of 6 slices with either 0.3 mM Mg²⁺ containing KR (LMKR; basal) or LMKR containing 10 mM NMDA and 1 mM glycine at 37 C for 30 minutes.

The incubation mixture was aerated with 95% O₂/5% CO₂ every 10 min for 1 minute during the stimulation. Ligand stimulation was terminated by the addition of 1 ml of ice-cold Ca²⁺-free K-R containing mixture of protein phosphatase inhibitors, 0.5 mM EGTA and 0.1 mM EDTA. After harvesting, tissues were briefly centrifuged and homogenized in 0.25 ml of ice-cold immunoprecipitation buffer. The homogenates were centrifuged at 1000·g for 5 minutes (4 C) and the supernatant (post-mitochondrial fraction) was sonicated for 10 seconds on ice.

The proteins were solubilized in 0.5% digitonin, 0.2% sodium cholate and 0.5% NP-40 for 60 minutes at 4 C with end-over-end rotation. The resultant lysates were cleared by centrifugation at 50,000·g for 5 minutes and diluted with 0.75 ml of immunoprecipitation buffer. Protein concentrations were measured by Bradford method (Bio-Rad).

To determine the NMDARs association with PSD-95 as well as NMDAR signaling, the levels of NMDAR subunits, PSD-95 and NMDAR-associated signaling molecules were measured in anti-NR1 immunoprecipitates. In these studies, brain tissue lysates (100 mg) were immunoprecipitated overnight (about 18 hours) at 4° C. with 2 mg of immobilized anti-NR1 onto covalently conjugated protein A-agarose beads (Pierce-ENDOGEN). Anti-NR1 immunoprecipitates were incubated with 75 ml antigen elution buffer (Pierce-ENDOGEN) and 2% SDS for 2 minutes on ice, centrifuged to remove antibody-protein A-agarose complexes and neutralized immediately with 10 ml 1.5 M Tris buffer, pH 8.8 followed by addition of 65 ml 2×PAGE sample buffer and boiled for 5 minutes.

Seventy-five ml of the obtained eluates (50%) were size fractionated on 7.5% SDS-PAGE. Proteins were transferred to a nitrocellulose membrane and the levels of various NMDA receptor subunits, PSD-95, signaling proteins were measured using Western blotting with antibodies for PSD-95, nNOS, phospholipase C-g1, gPKC, pY⁴⁰²PyK2, pY⁴¹⁶Src or phosphotyrosine. The blots were stripped and re-probed with anti-NR1 to assess the immunoprecipitation efficiency and loading.

IR activation and signaling was initiated by incubation of tissue with either K-R (basal) or K-R containing 1 nM insulin at 37 C for 30 minutes. The incubation mixture was aerated with 95% O₂/5% CO₂ every 10 minutes for 1 minute during the stimulation. Ligand stimulation was terminated by the addition of 1 ml of ice-cold Ca²⁺-free K-R containing mixture of protein phosphatase inhibitors, 0.5 mM EGTA and 0.1 mM EDTA.

Tissues were briefly centrifuged and homogenized in 0.25 ml of ice-cold immunoprecipitation buffer. The homogenates were centrifuged at 1000·g for 5 minutes (4 C) and the supernatant (post-mitochondrial fraction) was sonicated for 10 seconds on ice. The proteins were solubilized in 0.5% digitonin, 0.2% sodium cholate and 0.5% NP-40 for 60 minutes at 4 C with end-over-end rotation. The resultant lysates were then cleared by centrifugation at 50,000·g for 5 minutes and diluted with 0.75 ml of immunoprecipitation buffer. Protein concentrations were measured by Bradford method (Bio-Rad).

To determine the IR activation and signaling, the levels of pY^{1150/1151}IRb and the level of IR signal transducer, IRS-1 were measured in anti-IRb immunoprecipitates. In these studies, brain tissue lysates (100 mg) were immunoprecipitated overnight (about 18 hours) at 4° C. with 2 mg of immobilized anti-IRb onto covalently conjugated protein A-agarose beads (Pierce-ENDOGEN).

Anti-IRb immunoprecipitates were incubated with 75 ml antigen elution buffer (Pierce-ENDOGEN) and 2% SDS for 2 minutes on ice, centrifuged to remove antibody-protein A-agarose complexes and neutralized immediately with 10 ml 1.5 M Tris buffer, pH 8.8 followed by addition of 65 ml 2×PAGE sample buffer and boiled for 5 minutes.

Seventy-five ml of the obtained eluates (50%) were then size fractionated on 7.5% SDS-PAGE. Proteins were transferred to nitrocellulose membrane and the levels of pY^{1150/1151}IRb and IRS-1 proteins were measured using Western blotting with antibodies for pY^{1150/1151}IRb and IRS-1. The blots were stripped and re-probed with anti-IRb to assess the immunoprecipitation efficiency and loading.

7) Assessment of Cytokine Levels

Parietal cortices (about 10 mg) derived from (1) vehicle-treated sham, (2) compound C0105 treated sham, (3) vehicle-treated ICV Ab₄₂, and (4) compound C0105 treated ICV Ab₄₂ mice were first thawed slowly (−80° C. to −20° C. to −4° C.), homogenized in 100 ml of ice-cold homogenization medium (25 mM HEPES, pH 7.5; 50 mM NaCl, mixture of protease and protein phosphatase inhibitors) by sonication and then solubilized with 0.5% polyoxyethylene (40) nonyl phenyl ether (NP-40), 0.2% Na cholate and 0.5% digitonin at 4° C. for 1 hour with end-over-end shaking. Following centrifugation, the resultant lysate was then dilute with 500 ml (total volume 600 ml) and used as the source of cytokines.

To determine the levels of cytokines in these tissues, 0.5 mg/well biotinylated mouse monoclonal anti-TNF-a, anti-IL-6 and anti-IL-1b were coated onto streptavidin-coated plates (Reacti-Bind™ NeutrAvidin™ High binding capacity coated 96-well plate; Thermo Scientific Pierce Protein Research Products; Rockford, Ill.). Plates were washed 3 times with ice-cold 50 mM Tris HCl (pH 7.4) and incubated at 30° C. with 100 ml of lysate derived from the above mentioned tissues for 1 hour.

Plates were washed 3 times with ice-cold 50 mM Tris HCl (pH 7.4) and incubated at 30° C. with 0.5 mg/well un-conjugated rabbit anti-TNF-a, anti-IL-6 and anti-IL-1b for 1 hour. After two washes with 50 mM Tris HCl (pH 7.4), each well was incubated in 0.5 mg/well FITC-conjugated anti-rabbit IgG (human and mouse absorbed) for 1 hour at 30° C. Plates were washed twice with 200 ml ice-cold Tris HCl, pH 7.4 and the residual FITC signals were determined by multimode plate reader, DTX880 (Beckman). Each lysate was surveyed twice.

8) Immunohistochemical Studies

Quantitative immunohistochemistry on consecutive 5-mm sections containing prefrontal cortex and entorhinal cortex/hippocampus was used to determine the levels of Ab₄₂ aggregates/plaques and neurofibrillary pathology (NFT and paired helical filament [PHF] immunoreactivity) using single labeling immunohistochemistry as described previously [[Wang et al., Biol Psychiatry 67:522-530 (2010)]; D'Andrea et azl., Histopathology 38:120-134 (2001); and Nagele et al., Neuroscience 110:199-211 (2002)]. One section was immunostained with anti-NFT or -PHF. The next (consecutive) section (often containing the same neuron) was immunostained with anti-Ab₄₂ antibodies to measure relative levels of accumulated Ab₄₂ peptide in neurons. The relative Ab₄₂ accumulation extents were compared among different cell types using a computer-assisted image analysis as described previously [Wang et al., J Biol Chem 275:5626-5632 (2000)].

Brain tissues were fixed at 4° C. in 0.15 M phosphate-buffered 10% formalin, pH 7.4 for 2 weeks, paraffin embedded, serially sectioned at 5 mm, and processed for brightfield immunohistochemistry as described. The Ab₄₂ immunoreactivity was absent when pre-absorbing anti-Ab₄₂ with Ab₄₂ but not Ab₄₂₋₁. Specimens were examined using a Nikon FXA microscope with a Princeton Instruments CCD camera and recorded digitally.

Relative intensities of the NFT/PHF and Ab₄₂ immunoreactivity were measured and compared among similar and different cell types using Image-Pro® Plus (MediaCybernetics, Inc.; Bethesda, Md.) and Metamorph® software (Molecular Devices, Inc.; Sunnyvale, Calif.) as described previously [D'Andrea et al., Histopathology 38:120-134 (2001)]. The correlations between the amount of NFT/PHF immunoreactivity and Ab₄₂-positive material accumulated within mature neurons were also determined.

### Postmortem Tissue

This study protocol conformed to the Declaration of Helsinki: Ethical Principles for Biomedical Research Involving Human Beings (the 4^{th} amendment) as reflected in a prior approval by the City College of New York and City University of New York Medical School human research committee. The participants had a uniform clinical evaluation that included a medical history, complete neurological examination, cognitive testing including Mini-Mental state examination and other cognitive tests on episodic memory, semantic memory and language, working memory, perceptual speed, and visuospatial ability as well as psychiatric rating. Based on this information, subjects received AD diagnoses based on NINCDS-ADRDA criteria [Mckhann et al., Neurology 34, 939-944 (1984)].

Postmortem brain tissues (frontal cortex=FCX) from patients with clinically diagnosed sporadic AD and control tissues from normal, age-matched, neurologically normal individuals were obtained from the Harvard Brain Tissue Resource Center (HBTRC, Belmont, Mass.) and UCLA Brain Tissue Resource Center (UBTRC, Los Angeles, Calif.). Both HBTRC and UBTRC are supported in part by Public Health Service grants from the National Institute of Health. The postmortem time intervals for collecting these brains were £13 hours (mean postmortem intervals for collection of AD and control brain samples were 6.0±0.9 hours and 5.8±0.8 hours, respectively).

Diagnostic neuropathological examination was conducted on fixed sections stained with hematoxylin and eosin stain and with modified Bielschowsky silver staining [Yamamoto et al., Neuropathol Appl Neurobiol 12, 3-9 (1986)] to establish any disease diagnosis according to the criteria defined by the National Institute on Aging and the Reagan Institute Working Group on Diagnostic Criteria for the Neuropathological Assessment of AD [Hyman et al., J Neuropathol Exp Neurol 56, 1095-1097 (1997)] and brain tissue from age-matched controls was similarly screened. The presence of both neuritic (amyloid) plaques and neurofibrillary tangles in all AD brains was confirmed by Nissl and Bielschowsky staining as well as characterized immunohistochemically with anti-Ab₄₂ and -NFT staining in frontal and entorhinal cortex as well as hippocampus as described previously ([Wang et al., J Neurochem 75, 1155-1161 (2000)].

Control tissues exhibited no gross and minimal, localized microscopic neuropathology of AD (0-3 neuritic plaques/10× field and 0-6 NFTs/10× field in hippocampus). One gram blocks of FCX were dissected using a band saw from fresh frozen coronal brain sections maintained at −80° C. These blocks were derived from Brodmann areas 10 and/or 46. All postmortem tissues were identified by an anonymous identification number, and studies were performed as a best matched pair without knowledge of clinical information.

The Assessment of Test Compound Effects on Ab₄₂ Affinity for a7nAChRs

To determine the compound effect on Ab₄₂ affinity for the a7nAChRs, 200 mg of synaptosomes prepared from control subjects were biotinylated. The biotinylated synaptosomes were lysed by brief sonication in hypertonic solutions and used as the tissue source to determine Ab₄₂ affinity for the a7nAChRs in the presence and absence of Compound C0105.

In Vitro Treatment of Brain Slices for the Assessment of Test Compound on a7nAChR-FLNA, TLR2-FLNA and Ab₄₂-a7nAChR Associations, Ca²⁺ Influx, NMDAR and IR Signaling

Postmortem frontal cortex tissues were gradually thawed (from −80° C. to −20° C.) and were sliced using a chilled McIlwain tissue chopper (200 mm×200 mm×3 mm). Approximately 20 mg of the brain slices were suspended in 1 ml of ice-cold oxygenated Kreb's-Ringer solution (K-R), containing 25 mM HEPES, pH 7.4, 118 mM NaCl, 4.8 mM KCl, 1.3 mM CaCl₂, 1.2 mM KH₂PO₄, 1.2 mM MgSO₄, 25 mM NaHCO₃, 10 mM glucose, 100 mM ascorbic acid, 50 mg/ml leupeptin, 0.2 mM PMSF, 25 mg/ml pepstatin A, and 0.01 U/ml soybean trypsin inhibitor and centrifuged briefly. Following two additional washes with 1 ml of ice-cold K-R, brain slices were suspended in 1 ml of K-R.

To determine whether exposure to exogenous Ab₄₂ increases a7nAChR-FLNA, TLR2-FLNA and Ab₄₂-a7nAChR association and causes Ab₄₂-induced a7nAChR and N-methyl-D-aspartate receptor (NMDAR) dysfunction, approximately 20 mg of frontal cortical slices from control subjects were incubated with 0.1 mM of Ab₄₂ at 37° C. for 1 hour. To test the effects of C0105 on Ab₄₂-incubated control and native AD tissues, Compound C0105 (0.1 and 1 nM) was added 10 minutes following 0.1 mM Ab₄₂. Incubation continued for 1 hour in the dark to minimize light destruction of the test agents. The incubation mixture in a total incubation volume of 0.5 ml was aerated with 95% O₂/5% CO₂ every 15 minutes for 1 minute during the incubation. Reaction was terminated by the addition of 1.5 ml of ice-cold Ca²⁺-free K-R. Tissue slices were harvested by a brief centrifugation and used as the tissue sources for various assays.

To assess the effects of various a7nAChR agents on a7nAChR-FLNA linkages, about 20 mg of FCX from control subjects was incubated with 1 mM nicotine, PNU282987, a-bungarotoxin, methyllycaconitine, galantamine, memantine, and Ab₄₀ along with 0.1 mM Ab₄₂. Incubation continued for 1 hour in the dark. The incubation mixture in a total incubation volume of 0.5 ml was aerated for 1 minute every 15 minutes with 95% O₂/5% CO₂. The reaction was terminated by the addition of 1.5 ml of ice-cold Ca²⁺-free K-R, and slices were collected by a brief centrifugation.

Separately, the compound effect on a7nAChR-FLNA, TLR2-FLNA and Ab₄₂-a7nAChR complex levels were determined after incubation with 0.1 and 1 nM compounds in matching Krebs-Ringer and Ab₄₂-incubated synaptosomes from control subjects and Krebs-Ringer incubated Alzheimer's disease patients. The levels of a7nAChR-FLNA, TLR2-FLNA and Ab₄₂-a7nAChR complexes in the obtained synaptosomes were measured by co-immunoprecipitation method as described below that has been published [Wang et al., J Neurosci 35, 10961-10973 (2009)].

Assessment of a7nAChR-FLNA, TLR2-FLNA and Ab₄₂-a7nAChR Association by Co-Immunoprecipitation

Two-hundred mg of synaptosomes are pelleted by centrifugation and then solubilized by brief sonication in 250 ml of immunoprecipitation buffer (25 mM HEPES, pH 7.5; 200 mM NaCl, 1 mM EDTA, 50 mg/ml leupeptin, 10 mg/ml aprotinin, 2 mg/ml soybean trypsin inhibitor, 0.04 mM PMSF, 5 mM NaF, 1 mM sodium vanadate, 0.5 mM b-glycerophosphate and 0.1% 2-mercaptoethanol containing 0.5% digitonin, 0.2% sodium cholate and 0.5% NP-40 and incubated at 4° C. with end-to-end shaking for 1 hour. Following dilution with 750 ml of ice-cold immunoprecipitation buffer and centrifugation (4° C.) to remove insoluble debris, the a7nAChR-/LR4-FLNA and Ab₄₂-a7nAChR complexes in the lysate are isolated by immunoprecipitation with 16 hours of incubation at 4° C. with immobilized rabbit anti-FLNA (1 mg)—and anti-Ab₄₂ antibodies (1 mg)—protein A-conjugated agarose beads, respectively.

The resultant immunocomplexes were pelleted by centrifugation at 4° C. After three washes with 1 ml of ice-cold phosphate-buffered saline (PBS) (pH 7.2) and centrifugation, the isolated a7nAChR-/TLR2-FLNA and Ab₄₂-a7nAChR complexes are solubilized by boiling for 5 minutes in 100 ml of SDS-PAGE sample preparation buffer (62.5 mM Tris-HCl, pH 6.8; 10% glycerol, 2% SDS; 5% 2-mercaptoethanol, 0.1% bromophenol blue). The content of a7nAChRs in 50% of the obtained anti-Ab₄₂ immunoprecipitate was determined by Western blotting with monoclonal anti-a7nAChR antibodies. In the assay for determining Ab₄₂-a7nAChR complex level, immobilized rabbit anti-actin (0.5 mg)—protein A-conjugated agarose were added together with anti-Ab₄₂ in the co-immunoprecipitation process.

The content of b-actin in resultant immunoprecipitates is then analyzed by immunoblotting using monoclonal anti-b-actin to illustrate even immunoprecipitation efficiency and loading. In the assay for determining a7nAChR-/TLR2-FLNA complex levels, the blots obtained are stripped and re-probed with monoclonal anti-FLNA for assessing immunoprecipitation efficiency and loading.

### Assessment of Ca²⁺ Influx in Synaptosomes as a Functional Measurement of the Compounds

NMDAR-, a7nAChR- and voltage-gated calcium channel-mediated [⁴⁵Ca²⁺] influx were studied using synaptosomes prepared from postmortem frontal cortical slices from control and AD subjects. In brief, brain synaptosomes (100 mg for postmortem study) were incubated at 37 C for 5 minutes in oxygenated 0.3 mM Mg²⁺ K-R (low Mg²⁺ K-R, LMKR) containing 5 mM ⁴⁵Ca²⁺ (10 Ci/mmol) followed by incubation with vehicle, 0.1-10 mM PNU 282987, a specific a7nAChR agonist, or 0.1-10 mM NMDA+1 mM glycine for 5 minutes or 65 mM K⁺ (made with isomolar replacement of Na⁺) for 1 minute in a total incubation volume of 0.5 ml. The reaction was terminated by addition of 0.5 ml ice-cold Ca²⁺-free K-R containing 0.5 mM EGTA and centrifugation at 4° C. After two additional washes, ⁴⁵Ca²⁺ contents in synaptosomes were assessed using scintillation spectrometry (Beckman). The background ⁴⁵Ca²⁺ was estimated using hypotonically lysed synaptosomes. The absolute Ca²⁺ influx was calculated by subtracting the background ⁴⁵Ca²⁺ count. The percent increase in Ca²⁺ influx was calculated as % [(drug-treated−vehicle)/vehicle].

NMDAR Signaling and Association with PSD-95

NMDAR signaling and their interaction with synaptic anchoring protein, PSD-95 were compared in K-R and Compound C0105 (1 nM)-exposed frontal cortical slices from control and AD subjects. NMDAR activation and signaling were initiated by incubation of approximately 10 mg of in vitro treated brain slices with either LMKR (basal) or LMKR containing 10 mM NMDA and 1 mM glycine at 37 C for 30 minutes. The incubation mixture was aerated with 95% O₂/5% CO₂ every 10 minutes for 1 minute during the stimulation. Ligand stimulation was terminated by the addition of 1 ml of ice-cold Ca²⁺-free K-R containing 0.5 mM EGTA and 0.1 mM EDTA.

Brain slices were harvested by a brief centrifugation and were homogenized in 0.25 ml of ice-cold immunoprecipitation buffer. The homogenates were centrifuged at 1000·g for 5 minutes (4 C) and the supernatant (post-mitochondrial fraction) is sonicated for 10 seconds on ice. The proteins are solubilized in 0.5% digitonin, 0.2% sodium cholate and 0.5% NP-40 for 60 minutes at 4 C with end-over-end rotation. The resultant lysates are then cleared by centrifugation at 50,000·g for 5 minutes and diluted with 0.75 ml of immunoprecipitation buffer. Protein concentrations are measured by Bradford method (Bio-Rad).

To determine the NMDAR signaling and the NMDAR complexes association with PSD-95 [also known as Disks large homolog 4 (DLH4)], the levels of NMDAR subunits, PSD-95 and NMDAR-associated signaling molecules were measured in anti-NR1 immunoprecipitates. Two NR1 and two NR2 protein subunits form the heterotetramer NMDA receptor.

In these studies, brain slice lysates (200 mg) were immunoprecipitated overnight (about 18 hours) at 4° C. with 2 mg of immobilized anti-NR1 onto covalently conjugated protein A-agarose beads (Pierce-ENDOGEN). Anti-NR1 immunoprecipitates were incubated with 75 ml antigen elution buffer (Pierce-ENDOGEN) and 2% SDS for 2 minutes on ice, centrifuged to remove antibody-protein A-agarose complexes and neutralized immediately with 10 ml 1.5 M Tris buffer, pH 8.8, followed by addition of 65 ml 2×PAGE sample buffer and boiled for 5 minutes. Seventy-five ml of the obtained eluates (50%) were then size fractionated on 7.5% SDS-PAGE. Proteins were transferred to nitrocellulose membrane and the levels of various NMDA receptor subunits, PSD-95, signaling proteins were measured using Western blotting with antibodies for NR1, PSD-95, nNOS, phospholipase C-g1, gPKC, pY⁴⁰²PyK2, pY⁴¹⁶Src or phosphotyrosine. The blots were stripped and re-probed with anti-NR1 or -NR2A/-NR2B to assess the loading as appropriate.

### IR Activation and Signaling

IR signaling was compared in K-R and compound C0105-exposed frontal cortical slices from control and AD subjects. IR activation and signaling were initiated by incubation of approximately 10 mg of in vitro treated brain slices with either KR (basal) or KR containing 1 nM insulin at 37 C for 30 minutes. The incubation mixture was aerated with 95% O₂/5% CO₂ every 10 minutes for 1 minute during the stimulation. Ligand stimulation was terminated by the addition of 1 ml of ice-cold Ca²⁺-free K-R containing 0.5 mM EGTA and 0.1 mM EDTA. Brain slices were harvested by a brief centrifugation and were homogenized in 0.25 ml of ice-cold immunoprecipitation buffer. The homogenates were centrifuged at 1000·g for 5 minutes (4 C) and the supernatant (post-mitochondrial fraction) is sonicated for 10 seconds on ice. The proteins are solubilized in 0.5% digitonin, 0.2% sodium cholate and 0.5% NP-40 for 60 minutes at 4 C with end-over-end rotation. The resultant lysates are then cleared by centrifugation at 50,000·g for 5 minutes and diluted with 0.75 ml of immunoprecipitation buffer. Protein concentrations are measured by Bradford method (Bio-Rad).

To determine the IR signaling, the levels of pY^{1150/1151}- and pY⁹⁷²-IRs as well as insulin receptor substrate (IRS)-1 recruited to IR were measured in anti-IRb immunoprecipitates. In these studies, brain slice lysates (200 mg) were immunoprecipitated overnight (about 18 hours) at 4° C. with 2 mg of immobilized anti-IRb onto covalently conjugated protein A-agarose beads (Pierce-ENDOGEN). Anti-IRb immunoprecipitates were incubated with 75 ml antigen elution buffer (Pierce-ENDOGEN) and 2% SDS for 2 minutes on ice, centrifuged to remove antibody-protein A-agarose complexes and neutralized immediately with 10 ml 1.5 M Tris buffer, pH 8.8 followed by addition of 65 ml 2×PAGE sample buffer and boiled for 5 minutes. Seventy-five ml of the obtained eluates (50%) were then size fractionated on 7.5% SDS-PAGE. Proteins were transferred to nitrocellulose membrane and the levels of activated IR (pY^{1150/1151} and pY⁹⁷²) and IRS-1 recruited were measured using Western blotting with antibodies for pY^{1150/1151}-IRb, pY⁹⁷²-IRb or IRS-1. The blots were stripped and re-probed with anti-IRb to assess the loading as appropriate.

### Western Blot Analysis

Solubilized immunoprecipitates derived from co-immunoprecipitation assays were separated by either 7.5 or 10% SDS-PAGE and then electrophoretically transferred to nitrocellulose membranes. The membranes were washed with PBS and blocked overnight (about 18 hours) at 4 C with 10% milk in PBS with 0.1% Tween®-20 (PBST). Following three 5-minute washes with 0.1% PBST, the membranes were incubated at room temperature for 2 hours with antibody of choice at 1:500-1:1,000 dilutions. After three 2-minute washes in 0.1% PBST, membranes were incubated for 1 hour with anti-species IgG-HRP (1:5,000 dilution) and washed with 0.1% PBST three times, 2-minutes each. Immunoreactivity was visualized by reacting with chemiluminescent reagent (Pierce-ENDOGEN) for exactly 5 minutes and immediately exposing to X-ray film. Specific bands were quantified by densitometric scanning (GS-800 calibrated densitometer, Bio-Rad Laboratories).

Effects on Release of Pro-Inflammatory Cytokines (IL-1b, IL-6 and TNFa) Induced from Primary Human Astrocytes by Contact with Ab₄₂ and LPS

Human astrocytes express both the TLR2 and TLR2 cell surface receptors. Ab₄₂ and LPS each bind to and activate the TLR2 signaling pathway resulting in the release of pro-inflammatory cytokines such as IL-1b, IL-6 and TNFa, as is shown in other studies discussed herein. See also, Liu et al., J Immunol 188:1098-1107 (2012); and McIsaac et al., J Leukoc Biol 92:977-985 (2012).

### Experimental Design:

A primary astrocyte culture was prepared according to the provider (Lonza). The adherent astrocytes were trypsinized by 0.25% trypsin-EDTA, then collected and sub-cultured in 12-well plate (1.2 ml/well). When the cells were 80-85% confluent, cells were treated in an incubator under 5% CO₂ with 100 fM, 10 pM or 1 nM Compound C0105 immediately followed by the addition of Ab₄₂ (0.1 mM) and LPS (1 mg/ml); i.e., simultaneously adding the insulting ligand and Compound C0105 to the cells. Vehicle groups were treated with 0.1% DMSO only. Incubation continued for 24 hours post addition. Culture medium was used as the blank (non-treat) and the levels of cytokines, TNF-a, IL-6 and IL-1b in 200 ml of culture medium were determined. Each well was sampled once.

To determine the effect of Compound C0105 on cytokine release from human astrocytes, 0.5 mg/well biotinylated mouse monoclonal anti-TNF-a, -IL-6 and -IL-1b were coated onto streptavidin-coated plates (Reacti-Bind™ NeutrAvidin™ High binding capacity coated 96-well plates). Plates were washed 3 times with ice-cold 50 mM Tris HCl (pH 7.4) and incubated at 30° C. with 200 ml medium derived from the above-mentioned conditions. Plates were washed 3 times with ice-cold 50 mM Tris HCl (pH 7.4) and incubated at 30° C. with 0.5 mg/well un-conjugated rabbit anti-TNF-a, -IL-6 and -IL-1b for 1 hour. After three 1-minute washes with 50 mM Tris HCl (pH 7.4), each well was incubated in 0.25 mg/well FITC-conjugated anti-rabbit IgG (human and mouse absorbed) for 1 hour at 30° C. Plates were washed twice with 200 ml ice-cold Tris HCl, pH 7.4 and the residual FITC signals were determined by multimode plate reader, DTX880 (Beckman).

### FLNA Affinity Binding Studies

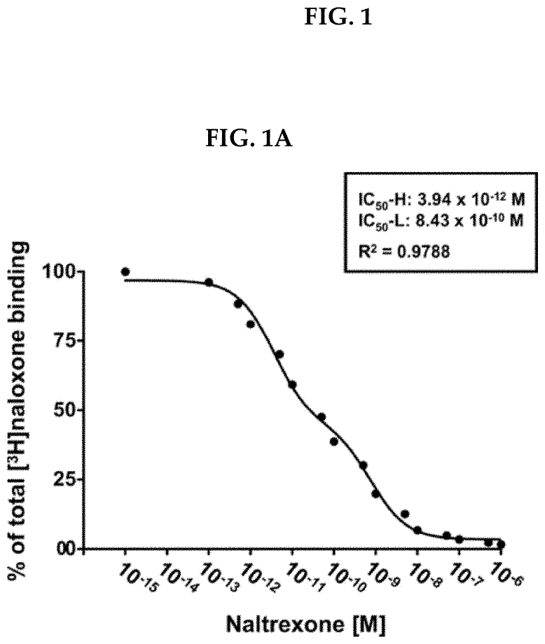

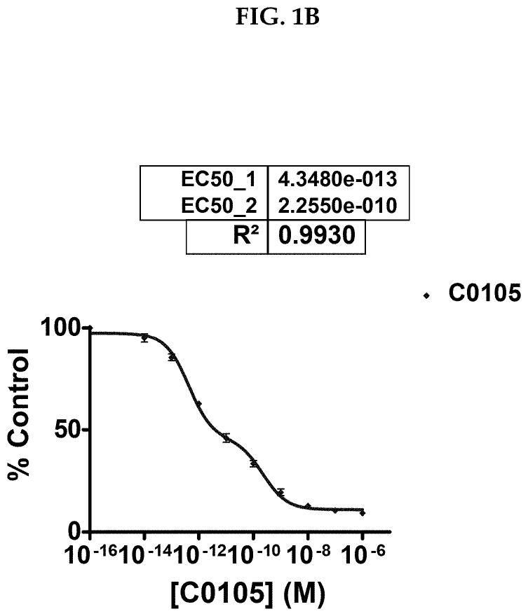

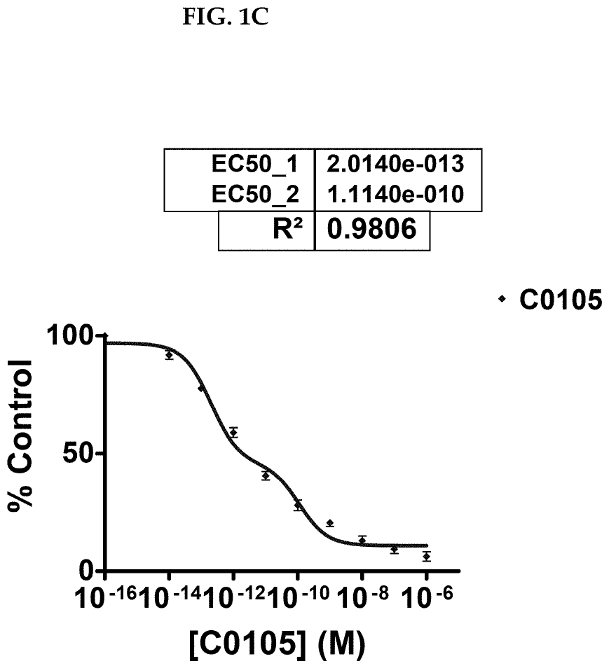

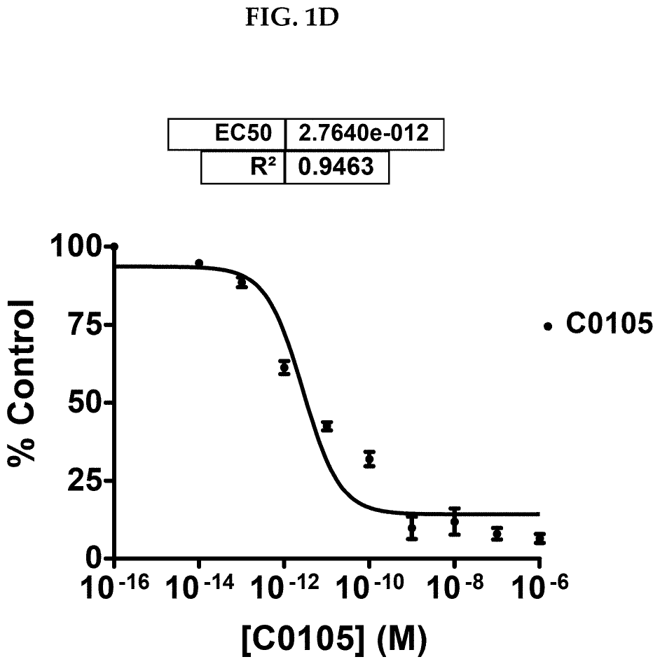

A series of binding studies using various compounds as ligand and FLNA or the FLNA pentamer peptide as the receptor. These studies were carried out in a generally similar manner using a competition (displacement) curve for the inhibition of [³H]NLX binding by in the presence of the ligand, and the results are shown in FIG. 1. Specifics of each study are set out below.

The competition (displacement) curve (FIG. 1A) for the inhibition of [³H]NLX binding by naltrexone to membranes from FLNA-expressing A7 (human melanocytic; ATCC CRL-2500) cells that are free of most receptors and particularly mu shows two affinity states with IC_{50-H} (high) of 3.94 picomolar and IC_{50-L} (low) of 834 picomolar. A nonlinear curve-fit analysis was performed using a competition equation that assumed two saturable sites for the naltrexone curve comprising of 16 concentrations ranging from 0.1 pM to 1 mM. Data are derived from six studies each using a different set of A7 cells.

The binding affinity of Compound C0105 for FLNA was similarly determined (FIG. 1B). Briefly, 100 mg of A7 cell membranes were incubated with 0.5 nM [³H]NLX in the presence of 0.01 nM-1 mM Compound C0105 at 30° C. for 60 minutes in 250 ml of the binding medium (50 mM Tris-HCl, pH 7.5; 100 mM NaCl; and protease and protein phosphatase inhibitors). Nonspecific binding was defined by 1 mM NTX. Reactions were terminated by rapid filtration through 3% BSA-treated glass microfiber binder free grade B (GF/B) membranes under vacuum. Filters were washed twice with 5 ml ice-cold binding medium, and [³H]NLX retained on the filters was measured by liquid scintillation spectrometry. The data obtained were analyzed using the GraphPad Software, Inc. (San Diego, Calif.) Prism program. Here, an IC_{50-H} of 0.43 picomolar and IC_{50-L} of 226 picomolar were determined. N=4.

The binding affinity of Compound C0105 for FLNA was similarly determined (FIG. 1C). Briefly, 200 mg of SK-N-MC (human neuroepithelioma; ATCC HTB-10) cell membranes that contain with both a7nAChR and mu-opioid receptors were incubated with 0.5 nM [³H]NLX in the presence of 1 mM DAMGO and 0.01 nM-1 mM Compound C0105 at 30° C. for 60 minutes in 250 ml of the binding medium (50 mM Tris-HCl, pH 7.5; 100 mM NaCl; and protease and protein phosphatase inhibitors). Nonspecific binding was defined by 1 mM NTX. Reactions were terminated by rapid filtration through 3% BSA-treated GF/B membranes under vacuum. Filters were washed twice with 5 ml ice-cold binding medium, and [³H]NLX retained on the filters was measured by liquid scintillation spectrometry. The data obtained were analyzed using the GraphPad Software, Inc. (San Diego, Calif.) Prism program. Here, an IC_{50-H} of 0.201 picomolar and IC_{50-L} of 111 picomolar were determined. N=4.

The binding affinity of Compound C0105 for the FLNA pentapeptide of positions 2561-2565 was also determined by a displacement assay (FIG. 1D). Briefly, 10 mg of N-terminal biotinylated FLNA pentapeptide peptide of FLNA positions 2561-2565 was incubated with 0.5 nM [³H]NLX in the presence of 0.01 nM-1 mM Compound C0105 at 30° C. for 60 minutes in 250 ml of the binding medium (50 mM Tris-HCl, pH 7.5; 100 mM NaCl; and protease and protein phosphatase inhibitors). Nonspecific binding was defined by 1 mM NTX. The reaction was terminated by addition of 1 ml of ice-cold binding medium. The [³H]NLX-bound biotinylated FLNA pentapeptide was trapped by incubation with 20 ml NeutrAvidin®-agarose (Thermo), followed by centrifugation. Following two 1.5 ml washes with PBS, the bound [³H]NLX was determined using scintillation spectrometry. The data obtained were analyzed using the GraphPad Software, Inc. (San Diego, Calif.) Prism program. Here, a single IC₅₀ value was obtained, as was expected for the 5-mer FLNA peptide of positions 2561-2565, and its value was 2.76 picomolar. N=4.

The data obtained in these studies illustrate the similar affinities exhibited between naloxone and illustrative Compound C0105 for FLNA. These data also illustrate the similarity in binding activity as a receptor shown between the intact FLNA molecule and the 5-mer FLNA peptide, and thereby validate the use of that 5-mer peptide as a surrogate for the complete molecule in the assays carried out herein.

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

1. A method of inhibiting one or more of a cell surface receptor-mediated immune response that comprises administering to immune cells having one or more of TLR2, RAGE, CCR5, CXR4 and CD4 cell surface receptors in recognized need thereof an effective amount of a compound or a pharmaceutically acceptable salt thereof selected from the group consisting of one or more of (a) Series C-1, Formula B, (b) Series C-2, Formula I, and (c) Series D, and said administration being carried out in the absence of a mu opioid receptor-(MOR-)binding effective amount of a separate MOR agonist or antagonist;
(a) a compound of Series C-1, Formula B has the structural formula 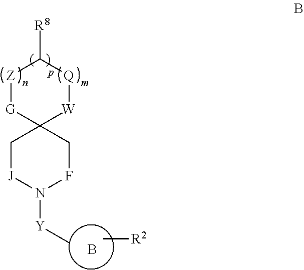
wherein
G and W are selected from the group consisting of NR²⁰, NR⁷, CH₂ and O, where R⁷ is H, C₁-C₁₂ hydrocarbyl, or C₁-C₁₂ hydrocarboyl and R²⁰ is a group X-circle A-R¹ as defined hereinafter;
X and Y are the same or different and are SO₂, C(O), CH₂, CD₂ (where D is deuterium), NHC(NH), OC(O), NHC(S) or NHC(O);
Q is CHR⁹ or C(O);
Z is CHR¹⁰ or C(O);
J and F are the same or different and are CH or CD (where D is deuterium);
each of m, n and p is zero or one and the sum of m+n+p is 2;
the circles A and B are the same or different aromatic or heteroaromatic ring systems that contain one ring or two fused rings;
groups R¹ and R² are the same or different and each is hydrogen or represents up to three substituents other than hydrogen that themselves can be the same or different, wherein each of those three groups, R^{1a-c} and R^{2a-c}, is separately selected from the group consisting of H, C₁-C₆ hydrocarbyl, C₁-C₆ hydrocarbyloxy, trifluoromethyl, trifluoromethoxy, C₁-C₇ hydrocarboyl (acyl), hydroxy-, trifluoromethyl- (—CF₃) or halogen-substituted C₁-C₇ hydrocarboyl, C₁-C₆ hydrocarbylsulfonyl, halogen, nitro, phenyl, cyano, carboxyl, C₁-C₇ hydrocarbyl carboxylate [C(O)O—C₁-C₇ hydrocarbyl], carboxamide [C(O)NR³R⁴] or sulfonamide [SO₂NR³R⁴],
wherein the amido nitrogen of either the carboxamide or sulfonamide has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, MAr, where M is —CH₂—, —O— or —N═N— and Ar is a single-ringed aryl group, and NR⁵R⁶,
wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur; and
R⁸, R⁹, and R¹⁰ are each H, or two of R⁸, R⁹, and R¹⁰ are H and one is a C₁-C₈ hydrocarbyl group that is unsubstituted or is substituted with up to three atoms that are the same or different and are oxygen or nitrogen atoms;
(b) a compound of Series C-2, Formula I has the structural formula 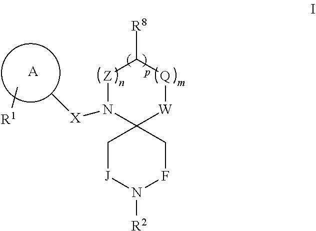
wherein
Q is CHR⁹ or C(O), Z is CHR¹⁰ or C(O), and only one of Q and Z is C(O);
each of m and n and p is zero or one and the sum of m+n+p is 2;
W is NR⁷ or O, where R⁷ and R² are the same or different and are H, C(H)ᵥ(D)ₕ where each of v and h is 0, 1, 2 or 3 and v+h=3, C(H)_{q}(D)ᵣ-aliphatic C₁-C₁₁ hydrocarbyl where each of q and r is 0, 1, or 2 and q+r=0, 1 or 2, (including aliphatic C₁-C₁₂ hydrocarbyl when q+r=0), aliphatic C₁-C₁₂ hydrocarbyl sulfonyl or aliphatic C₁-C₁₂ hydrocarboyl (acyl), and X-circle A-R¹ as defined hereinafter;
J and F are the same or different and are CH₂, CHD or CD₂ (where D is deuterium);
X is SO₂, C(O) or CH₂;
circle A is an aromatic or heteroaromatic ring system that contains a single ring or two fused rings;
R¹ is H or represents up to three substituents, R^{1a}, R^{1b}, and R^{1c}, that themselves can be the same or different, wherein each of those three groups, R^{1a-c}, is separately selected from the group consisting of H, C₁-C₆ hydrocarbyl, C₁-C₆ hydrocarbyloxy, C₁-C₆ hydrocarbyloxycarbonyl, trifluoromethyl, trifluoromethoxy, C₁-C₇ hydrocarboyl, hydroxy-, trifluoromethyl- or halogen-substituted C₁-C₇ hydrocarboyl, C₁-C₆ hydrocarbylsulfonyl, C₁-C₆ hydrocarbyloxysulfonyl, halogen, nitro, phenyl, cyano, carboxyl, C₁-C₇ hydrocarbyl carboxylate, carboxamide or sulfonamide, wherein the amido nitrogen in either amide group has the formula NR³R⁴ in which R³ and R⁴ are the same or different and are H, or C₁-C₄ hydrocarbyl, or R³ and R⁴ together with the depicted nitrogen form a 5-7-membered ring that optionally contains 1 or 2 additional hetero atoms that independently are nitrogen, oxygen or sulfur, MAr, where M is —CH₂—, —O— or —N═N— and Ar is a single-ringed aryl or heteroaryl group and NR⁵R⁶
wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur; and
R⁸ is H, or is a C₁-C₈ hydrocarbyl group that is unsubstituted or is substituted with up to three atoms that are the same or different and are oxygen or nitrogen atoms; and
(c) a compound of Series D corresponds in structure to the formula 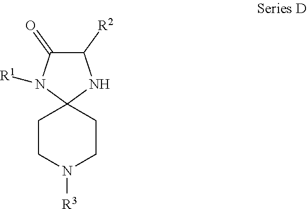
wherein
R¹ is hydrogen, a linear or branched unsubstituted or at least monosubstituted C₁₋₁₀ alkyl group that can comprise at least one heteroatom as a link; a linear or branched unsubstituted or at least monosubstituted C₂₋₁₀ alkenyl group that can comprise at least one heteroatom as a link; a linear or branched unsubstituted or at least monosubstituted C₂₋₁₀ alkynyl group that can comprise at least one heteroatom as a link; an unsubstituted or at least monosubstituted five-membered to fourteen-membered aryl group or heteroaryl group, that can be bonded via a linear or branched C₁₋₅ alkylene group that can comprise at least one heteroatom as a link; a —C═O)OR⁷ group that can be bonded via a linear or branched C₁₋₅ alkylene group;
R² is hydrogen, a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link, an unsubstituted or at least monosubstituted five-membered to fourteen-membered aryl or heteroaryl group, that can be bonded via a linear or branched C₁₋₅ alkylene group that can comprise at least one heteroatom as a link;
R³ is a —S(═O)₂—R⁴ group, a —C(═S)NH—R⁵ group, or a —C(═O)NH—R⁶ group;
R⁴ is an NR¹⁰R¹¹ group, a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link; an unsubstituted or at least monosubstituted five-membered to fourteen-membered aryl group or heteroaryl group, that can be bonded via a linear or branched unsubstituted or at least monosubstituted C₁₋₅ alkylene group that can comprise at least one heteroatom as a link and may be condensed with a five-membered or six-membered monocyclic ring system, an unsubstituted or at least monosubstituted C₃₋₈-cycloaliphatic group that can comprise at least one heteroatom as a ring member or that can be bonded via a linear or branched unsubstituted or at least monosubstituted C₁₋₅ alkylene group that can comprise at least one heteroatom as a link and that can be bridged by a linear or branched unsubstituted or at least monosubstituted C.sub.1-5 alkylene group;
R⁵ represents a linear or branched unsubstituted or at least monosubstituted C₁₋₁₀ alkyl group that can comprise at least one heteroatom as a link, a linear or branched unsubstituted or at least monosubstituted C₂₋₁₀ alkenyl group that can comprise at least one heteroatom as a link, a linear or branched unsubstituted or at least monosubstituted C₂₋₁₀ alkynyl group that can comprise at least one heteroatom as a link, an unsubstituted or at least monosubstituted five-membered to fourteen-membered aryl or heteroaryl group, that can be bonded via a linear or branched unsubstituted or at least monosubstituted C₁₋₅ alkylene group that can comprise at least one heteroatom as a link, an unsubstituted or at least monosubstituted C₃₋₈-cycloaliphatic group that can comprise at least one heteroatom as a ring member and that can be bonded via a linear or branched unsubstituted or at least monosubstituted C₁₋₅ alkylene group that can comprise at least one heteroatom as a link, a —C(═O)OR⁸ group or a —C(═O)OR⁹ group either of that can be bonded via a linear or branched C₁₋₁₀ alkylene group;
R⁶ represents an unsubstituted or at least monosubstituted five-membered to fourteen-membered aryl or heteroaryl group, which aryl or heteroaryl group may be bonded via a linear or branched unsubstituted or at least monosubstituted C₁₋₅ alkylene group that can comprise at least one heteroatom as a link, an unsubstituted or at least monosubstituted C₃₋₈-cycloaliphatic group that can comprise at least one heteroatom as a ring member, or that can be bonded via a linear or branched unsubstituted or at least monosubstituted C₁₋₅ alkylene group that can comprise at least one heteroatom as a link; and
R⁷, R⁸, R⁹, R¹⁰, and R¹¹, independently represent a linear or branched C₁₋₅ alkyl group, a linear or branched C₂₋₅ alkenyl group, or a linear or branched C₂₋₅ alkynyl group.

2. The method according to claim 1, wherein said compound exhibits less than about 80 percent the MOR stimulation provided by DAMGO at the same concentration.

3. The method according to claim 1, wherein said compound or a pharmaceutically acceptable salt thereof is present dissolved or dispersed in a pharmaceutically acceptable diluent as a pharmaceutical composition when administered.

4. The method according to claim 1, wherein said compound is a compound of Series C-2, Formula I, wherein J and F are both CH₂, p is one, and X is SO₂.

5. The method according to claim 4, wherein said compound corresponds in structure to a compound whose formula is shown below:

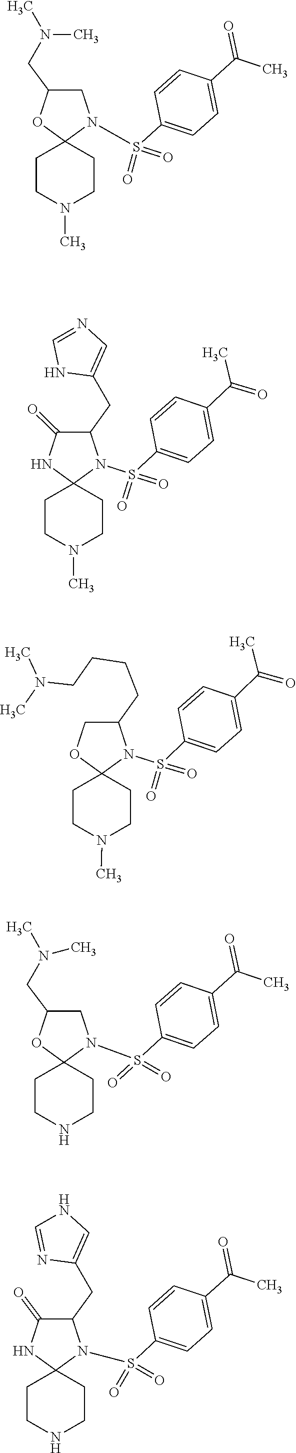

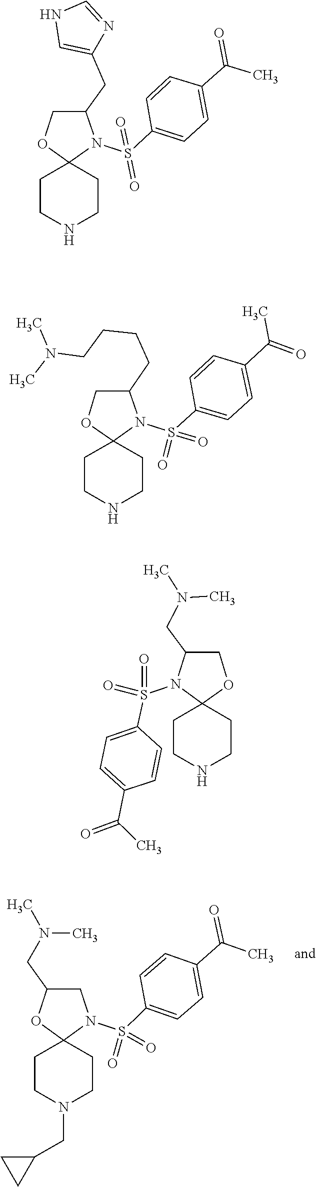

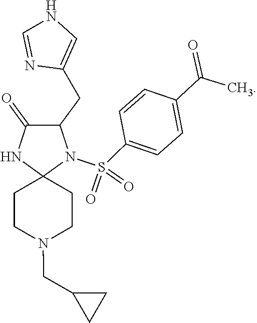 [SMILES: CC(=O)c1ccc(S(=O)(=O)N2C(Cc3c[nH]cn3)C(=O)NC23CCN(CC2CC2)CC3)cc1]

6. The method according to claim 1, wherein said compound is a compound of Series C-2 that corresponds in structure to the Formula II below: 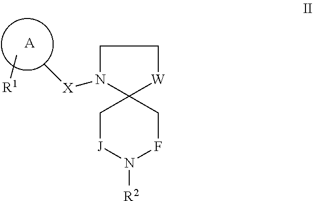
wherein J and F are the same or different and are CH₂, CHD or CD₂ (where D is deuterium).

7. The method according to claim 6, wherein said compound corresponds in structure to a compound whose formula is shown below:

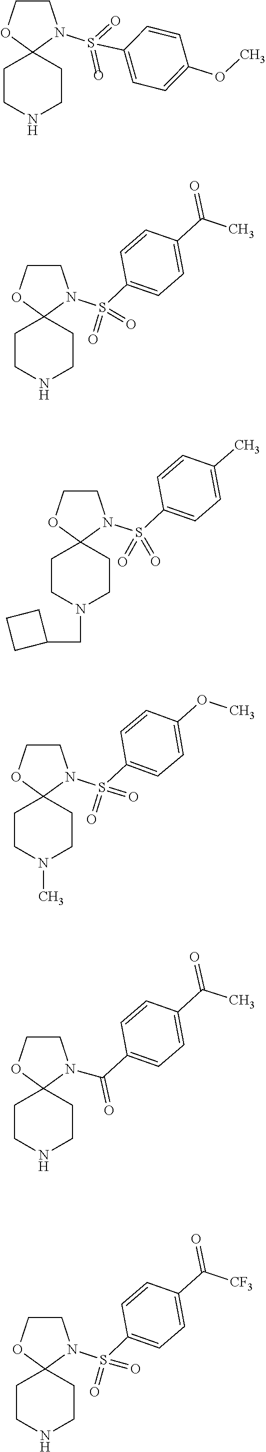

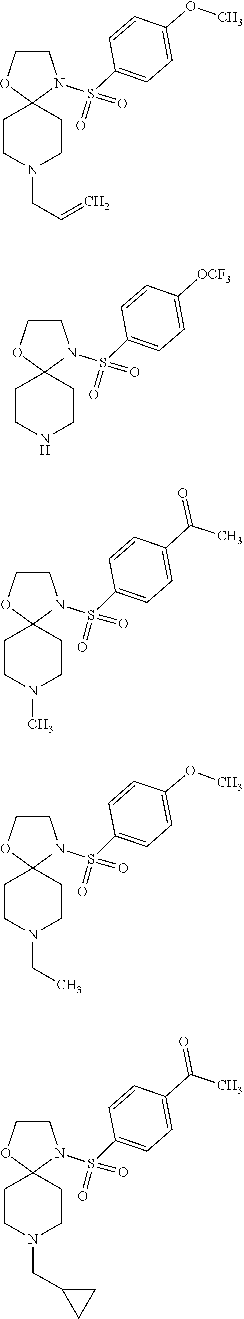 [SMILES: C=CCN1CCC2(CC1)OCCN2S(=O)(=O)c1ccc(OC)cc1 ; O=S(=O)(c1ccc(OC(F)(F)F)cc1)N1CCOC12CCNCC2 ; CC(=O)c1ccc(S(=O)(=O)N2CCOC23CCN(C)CC3)cc1 ; CCN1CCC2(CC1)OCCN2S(=O)(=O)c1ccc(OC)cc1 ; CC(=O)c1ccc(S(=O)(=O)N2CCOC23CCN(CC2CC2)CC3)cc1]

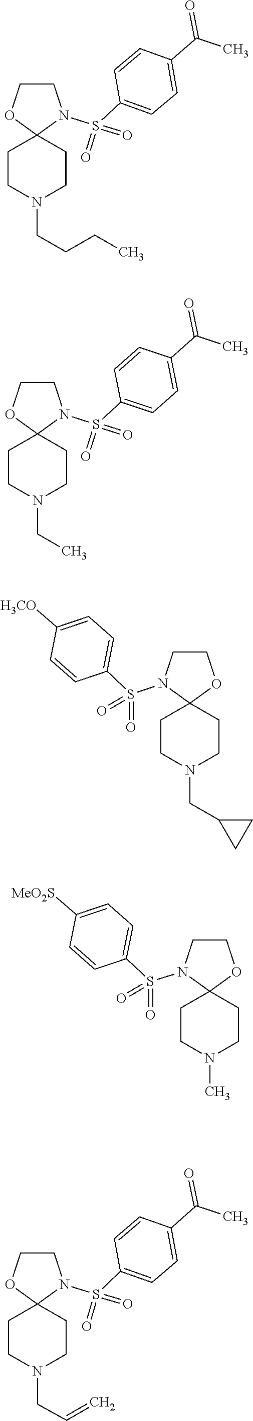 [SMILES: CCCCN1CCC2(CC1)OCCN2S(=O)(=O)c1ccc(C(C)=O)cc1 ; CCN1CCC2(CC1)OCCN2S(=O)(=O)c1ccc(C(C)=O)cc1 ; COc1ccc(S(=O)(=O)N2CCOC23CCN(CC2CC2)CC3)cc1 ; CN1CCC2(CC1)OCCN2S(=O)(=O)c1ccc(S(C)(=O)=O)cc1 ; C=CCN1CCC2(CC1)OCCN2S(=O)(=O)c1ccc(C(C)=O)cc1]

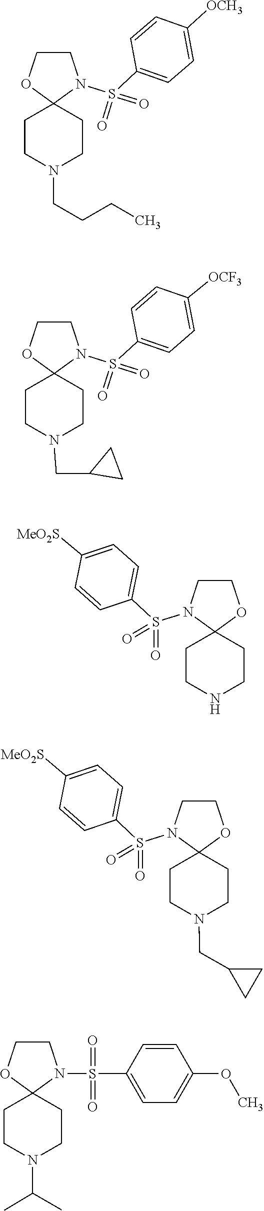 [SMILES: CCCCN1CCC2(CC1)OCCN2S(=O)(=O)c1ccc(OC)cc1 ; O=S(=O)(c1ccc(OC(F)(F)F)cc1)N1CCOC12CCN(CC1CC1)CC2 ; CS(=O)(=O)c1ccc(S(=O)(=O)N2CCOC23CCNCC3)cc1 ; CS(=O)(=O)c1ccc(S(=O)(=O)N2CCOC23CCN(CC2CC2)CC3)cc1 ; COc1ccc(S(=O)(=O)N2CCOC23CCN(C(C)C)CC3)cc1]

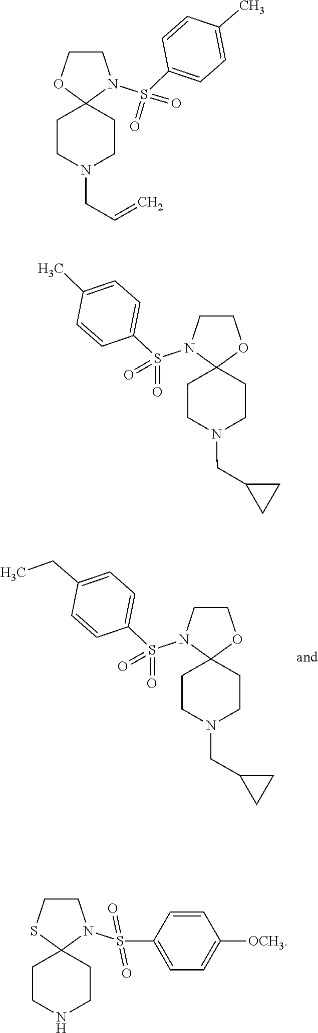

8. The method according to claim 1, wherein said compound is a compound of Series C-2 that corresponds in structure to the Formula III below: 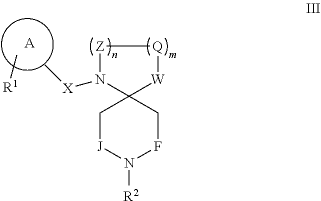
wherein J and F are the same or different and are CH₂, CHD or CD₂ (where D is deuterium); and
each of m and n is one.

9. The method according to claim 8, wherein said compound corresponds in structure to a compound whose formula is shown below:

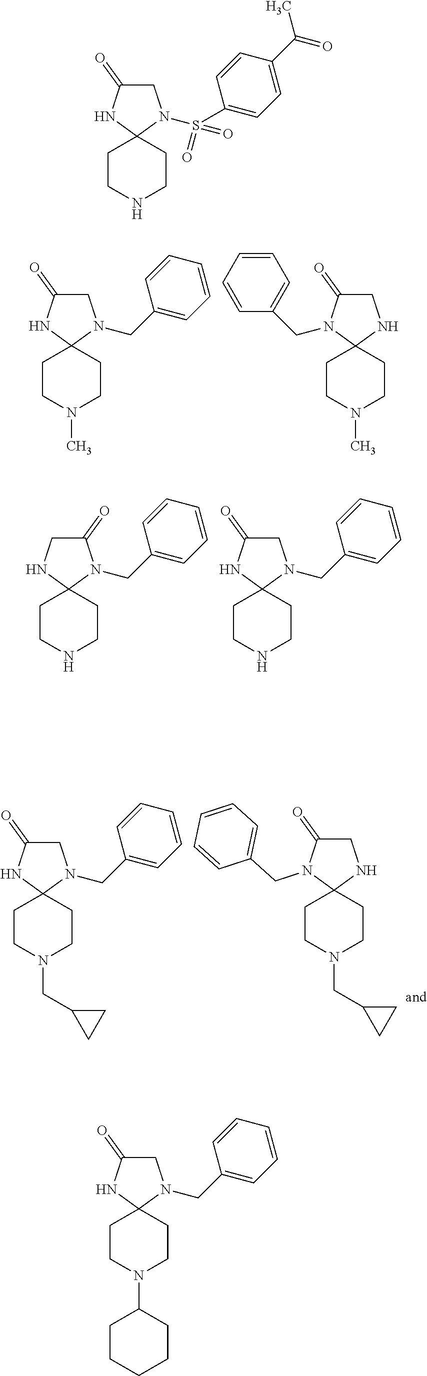

10. The method according to claim 1, wherein said compound is a compound of Series C-1 that corresponds in structure to the Formula I below: 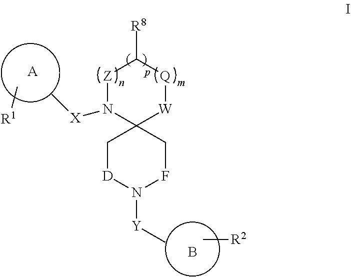
wherein
X and Y are the same or different and are SO₂, C(O), CH₂, CD₂ (where D is deuterium), NHC(NH), OC(O), NHC(S) or NHC(O);
W is NR⁷, CH₂, or O, where R⁷ is H, C₁-C₁₂ hydrocarbyl, or C₁-C₁₂ hydrocarboyl (acyl);
Q is CHR⁹ or C(O);
Z is CHR¹⁰ or C(O);
J and F are the same or different and are CH₂, CHD or CD₂ (where D is deuterium);
each of m, n and p is zero or one and the sum of m+n+p is 2 or 3; and
circles A and B are the same or different aromatic or heteroaromatic ring systems that contain one ring or two fused rings;
R¹ and R² are the same or different and each can be hydrogen or represent up to three substituents other than hydrogen that themselves can be the same or different (R^{1a}, R^{1b}, and R^{1c}, and R^{2a}, R^{2b}, R^{2c}) each of those six groups, R^{1a-c} and R^{2a-c}, is separately selected from the group consisting of H, C₁-C₆ hydrocarbyl, C₁-C₆ hydrocarbyloxy, trifluoromethyl, trifluoromethoxy, C₁-C₇ hydrocarboyl, hydroxy-, trifluoromethyl- or halogen-substituted C₁-C₇ hydrocarboyl, C₁-C₆ hydrocarbylsulfonyl, halogen, nitro, phenyl, cyano, carboxyl, C₁-C₇ hydrocarbyl carboxylate, carboxamide or sulfonamide
wherein the amido nitrogen of either group has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur,
MAr, where M is where M is —CH₂—, —O— or —N═N— and Ar is a single-ringed aryl group, and NR⁵R⁶
wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur; and
R⁸, R⁹, and R¹⁰ are each H, or two of R⁸, R⁹, and R¹⁰ are H and one is a C₁-C₈ hydrocarbyl group that is unsubstituted or is substituted with up to three atoms that are the same or different and are oxygen or nitrogen atoms.

11. The method according to claim 10, wherein said compound is a compound of Series C-1 that corresponds in structure to the Formula II below: 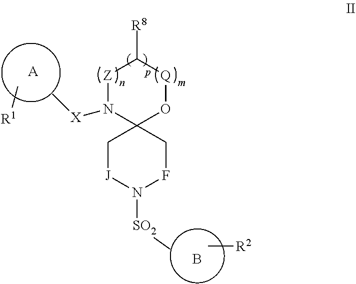
wherein
Q is CHR⁹ or C(O);
Z is CHR¹⁰ or C(O);
each of m, n and p is zero or one and the sum of m+n+p is 2 or 3;
J and F are the same or different and are CH₂, CHD or CD₂ (where D is deuterium);
circles A and B are the same or different aromatic or heteroaromatic ring systems;
R¹ and R² are the same or different and each can be hydrogen or represent up to three substituents other than hydrogen that themselves can be the same or different (R^{1a}, R^{1b}, and R^{1c}, and R^{2a}, R^{2b}, and R^{2c}), each of those six groups, R^{1a-c} and R^{2a-c}, is separately selected from the group consisting of H, C₁-C₆ hydrocarbyl, C₁-C₆ hydrocarbyloxy, C₁-C₆ hydrocarbyloxycarbonyl, trifluoromethyl, trifluoromethoxy, C₁-C₇ hydrocarboyl, hydroxy-, trifluoromethyl- or halogen-substituted C₁-C₇ hydrocarboyl, C₁-C₆ hydrocarbylsulfonyl, C₁-C₆ hydrocarbyloxysulfonyl, halogen, nitro, phenyl, cyano, carboxyl, C₁-C₇ hydrocarbyl carboxylate, carboxamide or sulfonamide,
wherein the amido nitrogen in either group has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur,
MAr, where M is —CH₂—, —O— or —N═N— and Ar is a single-ringed aryl group, and NR⁵R⁶
wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

12. The method according to claim 11, wherein said compound of Series C-1 corresponds in structure to a compound of Formula II shown below:

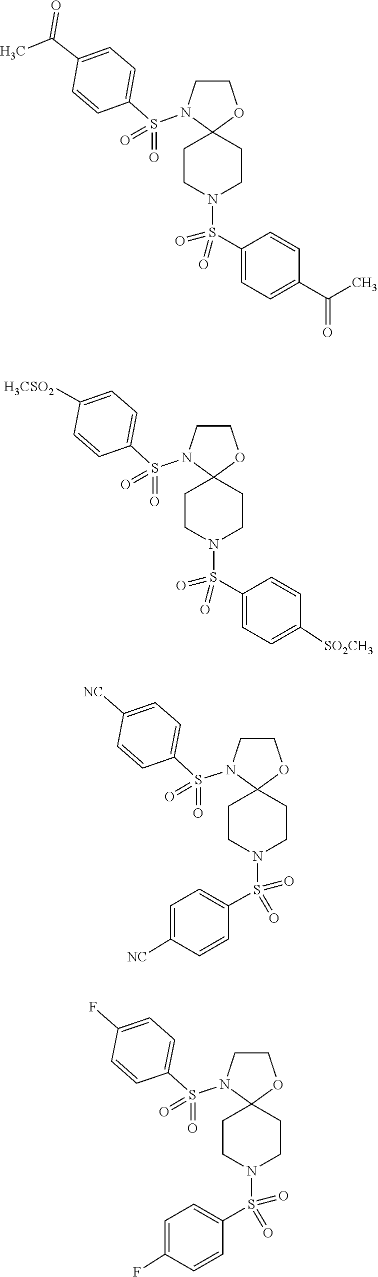

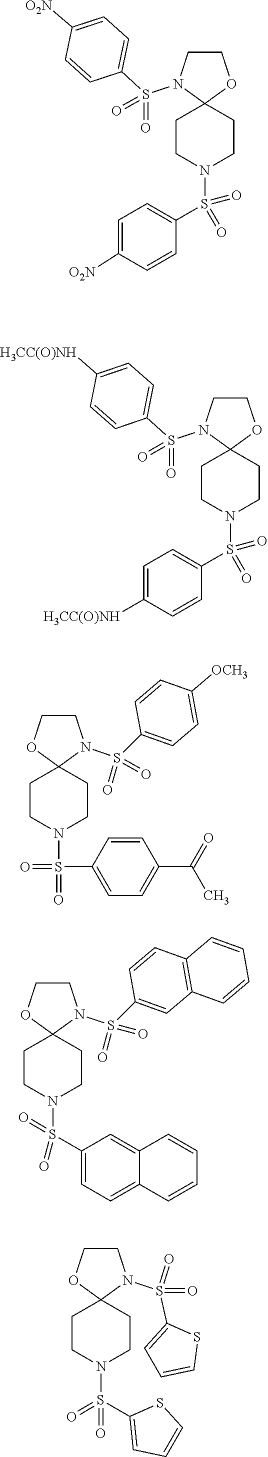

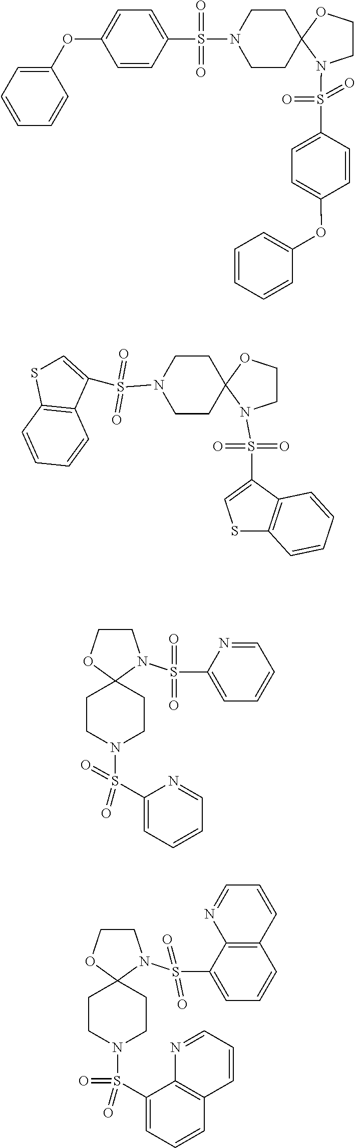 [SMILES: O=S(=O)(c1ccc(Oc2ccccc2)cc1)N1CCC2(CC1)OCCN2S(=O)(=O)c1ccc(Oc2ccccc2)cc1 ; O=S(=O)(c1csc2ccccc12)N1CCC2(CC1)OCCN2S(=O)(=O)c1csc2ccccc12 ; O=S(=O)(c1ccccn1)N1CCC2(CC1)OCCN2S(=O)(=O)c1ccccn1 ; O=S(=O)(c1cccc2cccnc12)N1CCC2(CC1)OCCN2S(=O)(=O)c1cccc2cccnc12]

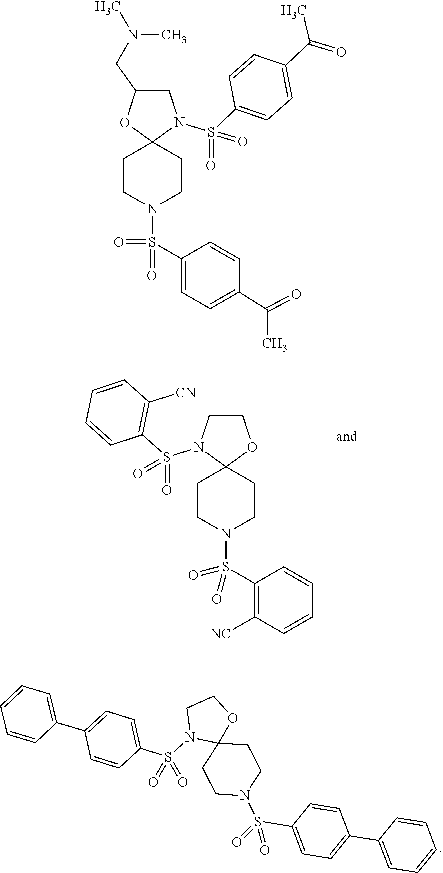 [SMILES: CC(=O)c1ccc(S(=O)(=O)N2CCC3(CC2)OC(CN(C)C)CN3S(=O)(=O)c2ccc(C(C)=O)cc2)cc1 ; N#Cc1ccccc1S(=O)(=O)N1CCC2(CC1)OCCN2S(=O)(=O)c1ccccc1C#N ; O=S(=O)(c1ccc(-c2ccccc2)cc1)N1CCC2(CC1)OCCN2S(=O)(=O)c1ccc(-c2ccccc2)cc1]

13. The method according to claim 10, wherein said compound is a compound of Series C-1 that corresponds in structure to the Formula III below: 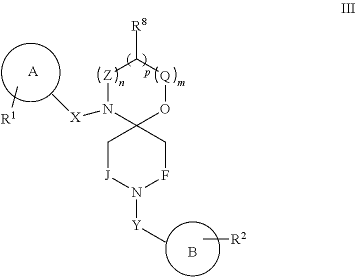
wherein
Q is CHR⁹ or C(O);
Z is CHR¹⁰ or C(O);
each of m, n and p is zero or one and the sum of m+n+p is 2 or 3;
J and F are the same or different and are CH₂, CHD or CD₂ (where D is deuterium);
X and Y are both CO, or X and Y are different and are SO₂, C(O), CH₂, CD₂ (where D is deuterium), NHC(NH), NHC(S) or NHC(O);
circles A and B are the same or different aromatic or heteroaromatic ring systems;
R¹ and R² are the same or different and each can be hydrogen or represent up to three substituents other than hydrogen that themselves can be the same or different (R^{1a}, R^{1b}, and R^{1c}, and R^{2a}, R^{2b}, and R^{2c}), each of those six groups, R^{1a-c} and R^{2a-c}, is separately selected from the group consisting of H, C₁-C₆ hydrocarbyl, C₁-C₆ hydrocarbyloxy, C₁-C₆ hydrocarbyloxycarbonyl, trifluoromethyl, trifluoromethoxy, C₁-C₇ hydrocarboyl, hydroxy-, trifluoromethyl- or halogen-substituted C₁-C₇ hydrocarboyl, C₁-C₆ hydrocarbylsulfonyl, C₁-C₆ hydrocarbyloxysulfonyl, halogen, nitro, phenyl, cyano, carboxyl, C₁-C₇ hydrocarbyl carboxylate, carboxamide or sulfonamide,
wherein the amido nitrogen in either group has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur,
MAr, where M is —CH₂—, —O— or —N═N— and Ar is a single-ringed aryl group, and NR⁵R⁶
wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

14. The method according to claim 13, wherein said compound of Series C-1 corresponds in structure to the compound of Formula III is shown below:

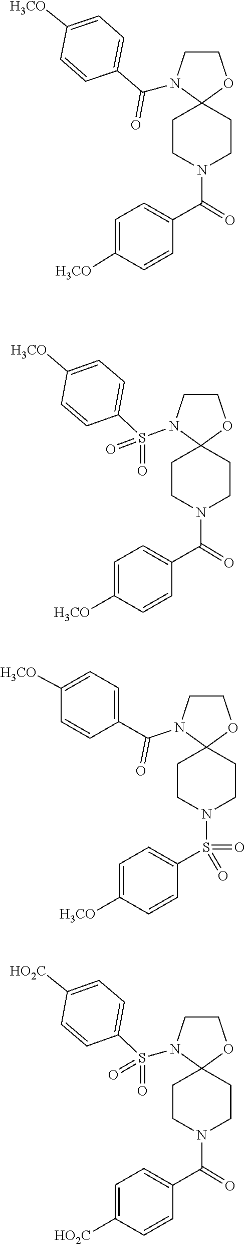 [SMILES: COc1ccc(C(=O)N2CCC3(CC2)OCCN3C(=O)c2ccc(OC)cc2)cc1 ; COc1ccc(C(=O)N2CCC3(CC2)OCCN3S(=O)(=O)c2ccc(OC)cc2)cc1 ; COc1ccc(C(=O)N2CCOC23CCN(S(=O)(=O)c2ccc(OC)cc2)CC3)cc1 ; O=C(O)c1ccc(C(=O)N2CCC3(CC2)OCCN3S(=O)(=O)c2ccc(C(=O)O)cc2)cc1]

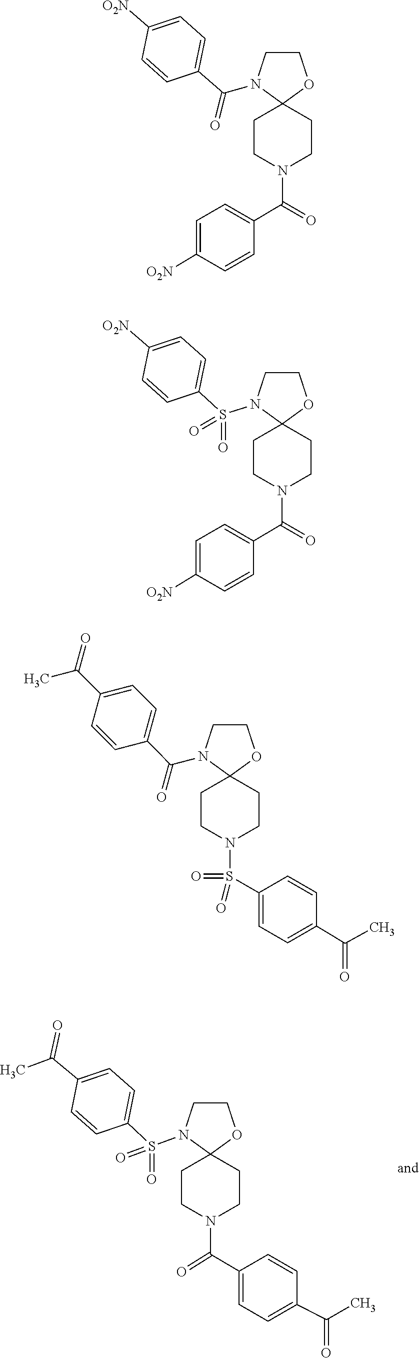

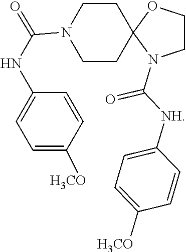 [SMILES: COc1ccc(NC(=O)N2CCC3(CC2)OCCN3C(=O)Nc2ccc(OC)cc2)cc1]

15. The method according to claim 10, wherein said compound is a compound of Series C-1 that corresponds in structure to the Formula IV below: 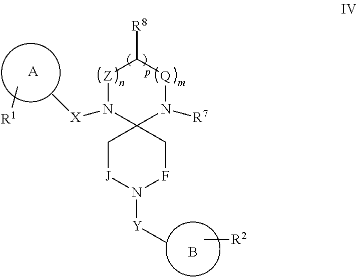
wherein
Q is CHR⁹ or C(O);
Z is CHR¹⁰ or C(O);
each of m, n and p is zero or one and the sum of m+n+p is 2 or 3;
J and F are the same or different and are CH₂, CHD or CD₂ (where D is deuterium);
X and Y are the same or different and are SO₂, C(O), CH₂, CD₂ (where D is deuterium), OC(O), NHC(NH), NHC(S) or NHC(O);
circles A and B are the same or different aromatic or heteroaromatic ring systems;
R¹ and R² are the same or different and each can be hydrogen or represent up to three substituents other than hydrogen that themselves can be the same or different (R^{1a}, R^{1b}, and R^{1c}, and R^{2a}, R^{2b}, and R^{2c}), each of those six groups, R^{1a-c} and R^{2a-c}, is separately selected from the group consisting of H, C₁-C₆ hydrocarbyl, C₁-C₆ hydrocarbyloxy, C₁-C₆ hydrocarbyloxycarbonyl, trifluoromethyl, trifluoromethoxy, C₁-C₇ hydrocarboyl, hydroxy-, trifluoromethyl- or halogen-substituted C₁-C₇ hydrocarboyl, C₁-C₆ hydrocarbylsulfonyl, C₁-C₆ hydrocarbyloxysulfonyl, halogen, nitro, phenyl, cyano, carboxyl, C₁-C₇ hydrocarbyl carboxylate, carboxamide or sulfonamide,
wherein the amido nitrogen in either group has the formula NR³R⁴ wherein R³ and R⁴ are the same or different and are H, C₁-C₄ hydrocarbyl, or R³ and R⁴ together with the depicted nitrogen form a 5-7-membered ring that optionally contains 1 or 2 additional hetero atoms that independently are nitrogen, oxygen or sulfur,
MAr, where M is —CH₂—, —O— or —N═N— and Ar is a single-ringed aryl group, and NR⁵R⁶
wherein R⁵ and R⁶ are the same or different and are H, C₁-C₄ hydrocarbyl, C₁-C₄ acyl, C₁-C₄ hydrocarbylsulfonyl, or R⁵ and R⁶ together with the depicted nitrogen form a 5-7-membered ring that optionally contains 1 or 2 additional hetero atoms that independently are nitrogen, oxygen or sulfur.

16. The method according to claim 15, wherein said compound of Series C-1 corresponds in structure to a compound of Formula IV as is shown below:

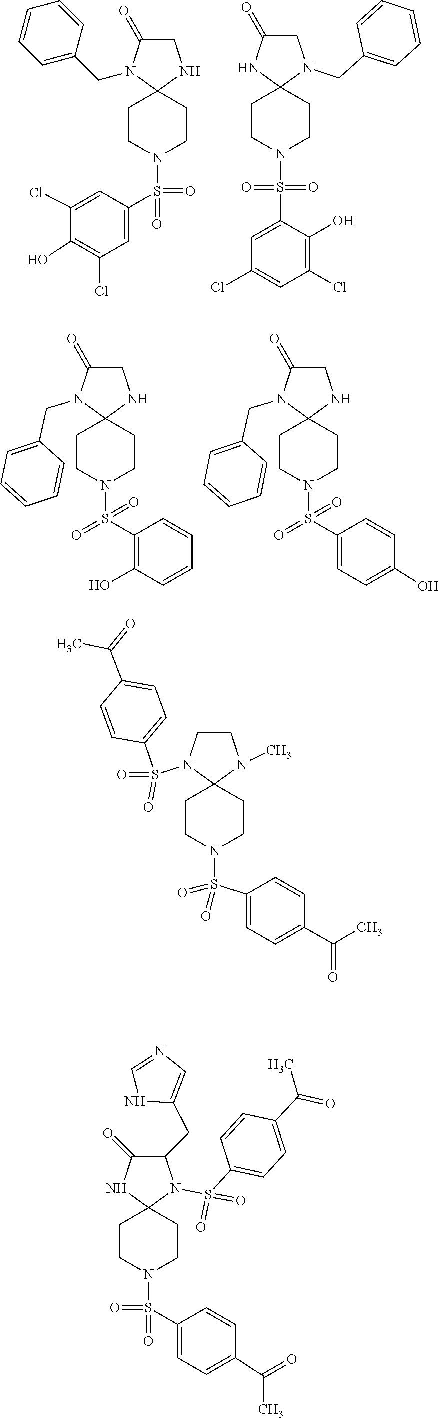

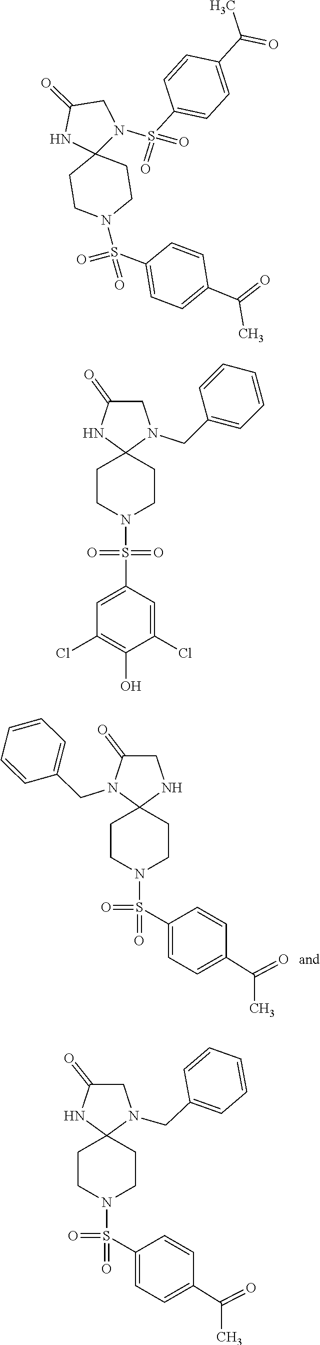

18. The method according to claim 1, wherein said compound is a compound of Series C-1 Formula B.

19. The method according to claim 18, wherein said compound is a compound of Series C-1 Formula B that corresponds in structure to the formula below:

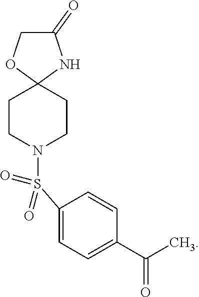 [SMILES: CC(=O)c1ccc(S(=O)(=O)N2CCC3(CC2)NC(=O)CO3)cc1]

20. The method according to claim 1, wherein said compound is a compound of Series D.

21. The method according to claim 20, wherein said compound corresponds in structure to compound shown below:

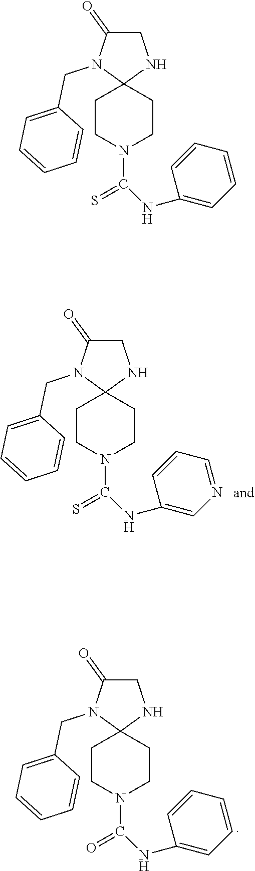 [SMILES: O=C1CNC2(CCN(C(=S)Nc3ccccc3)CC2)N1Cc1ccccc1 ; O=C1CNC2(CCN(C(=S)Nc3cccnc3)CC2)N1Cc1ccccc1 ; "and" ; O=C(Nc1ccccc1)N1CCC2(CC1)NCC(=O)N2Cc1ccccc1]

22. The method according to claim 1, wherein said administration is carried out a plurality of times.

23. The method according to claim 1, wherein said administration is carried out daily.

24. The method according to claim 23, wherein said administration is carried out multiple times daily.

25. The method according to claim 1, wherein said compound or a pharmaceutically acceptable salt thereof is present dissolved or dispersed in a pharmaceutically acceptable diluent as a pharmaceutical composition when administered.

26. The method according to claim 1, wherein said immune cells recognized need and having one or more of TLR2, RAGE, CCR5, CXR4 and CD4 cell surface receptors has a hyperinflammatory syndrome.

27. The method according to claim 1, wherein said hyperinflammatory syndrome is selected from one or more of the group consisting of secondary hemophagocytic lymphohistiocytosis (sHLH), acute respiratory distress syndrome (ARDS), cytokine storm, sepsis, non-infectious systemic inflammatory response syndrome (SIRS), hypotensive shock, multi-organ failure and macrophage activation syndrome (MAS).
